(19) United States
(12) Patent Application Publication
(10) Pub. No.: US 2025/0174299 A1
(43) Pub. Date: May 29, 2025

(54) METHODS FOR PEPTIDE SYNTHESIS

(71) Applicant: Achilles Therapeutics UK Limited, London (GB)

(72) Inventors: Lawrence Clark, London (GB); Maximilian Prinz zu Salm-Horstmar, London (GB); Andrew Kennedy, London (GB); Jessica Sayers, London (GB)

(21) Appl. No.: 18/841,187

(22) PCT Filed: Mar. 2, 2023
(86) PCT No.: PCT/EP2023/055383

§ 371 Date: Aug. 23, 2024

(30) Foreign Application Priority Data
Mar. 2, 2022 (GB) 2202880.7

(51) Int. Cl. G16B 15/30; G16B 15/20; G16B 30/00; G16B 40/30; G16H 20/17

(52) U.S. Cl. CPC G16B 15/30; G16B 15/20; G16B 30/00; G16B 40/30; G16H 20/17

(57) ABSTRACT

Methods for predicting the outcome of production of peptides are provided, as well as methods for producing peptides or providing products comprising peptides, which uses said methods. The methods comprise providing a primary amino acid sequence for one or more peptides, and Provide ore or more metrics predicting the outcome of production of the one or more peptides by chemical synthesis using a machine learning model that has been trained to predict one or more metrics characterising the outcome of production of peptides using training data comprising training peptide primary sequences and measured metrics characterising the outcome of production of the training peptides by chemical synthesis. The measured metrics are metrics obtained after completion of the chemical synthesis process. Systems and products for performing the methods are also described.

### FIELD OF THE DISCLOSURE

The present disclosure relates to methods for predicting the outcome of producing peptides by chemical synthesis, and methods for producing peptides by chemical synthesis that make use of such predictions. The present disclosure also relates to methods and compositions for the treatment of diseases which make use of produced peptides.

### BACKGROUND TO THE DISCLOSURE

Typical manufacturing of peptide by chemical synthesis in the so-called “solid phase peptide synthesis” (SPPS) process involves the stepwise formation of amide bonds between each successive amino acid in the nascent peptide immobilised on a solid support. Each addition requires multiple reactions (in particular, the deprotection of the last added amino-protected amino acid and the reaction with the carbonyl group of the next amino-protected amino acid, also referred to as coupling). Each such reaction proceeds with a particular yield, such that the overall yield of production of a peptide decreases with the length of the peptide. Additionally, the yield is often impacted by events such as aggregation, leading to incomplete peptides forming by-products that affect the purity of the final product (if a final product comprising a full length peptide can even be obtained). The factors that affect the purity and yield of the final product are poorly understood. Thus, peptide manufacturers typically perform extensive optimisation by expert-guided trial and error to produce desired peptides. Such a process is time, labour and cost consuming, and is often simply impractical for applications that require the production of varied panels of peptides.

An example of such an application is personalised immunotherapies. For example, peptides comprising mutations that are expressed by cancer cells (also referred to as “neoantigens”) can be used to produce vaccines or selectively expand T cells that recognise these cancer cells (see e.g. WO 2016/16174085). Some of these mutations may be common to multiple cancers (e.g. in the case of driver mutations), but many others will be specific to a particular individual's tumour. As a consequence, a personalised immunotherapy may be advantageous, and this may require the production of a different set of peptides for each patient to be treated. In order for such an approach to be viable, it must be practically possible to produce enough peptides with required characteristics for each patient, within a reasonable time frame. Thus, there is a need to identify which patient specific peptides can be produced with desired characteristics, without the heavy burden of optimisation that is currently required. More generally, there is a need for improved methods for predicting the outcome of peptide production by chemical synthesis.

### SUMMARY OF THE INVENTION

Peptide synthesis experts have informally known for many years that some peptides have so called “difficult sequences”. However, what characterises these sequences is a matter of debate in the community. Milton et al. (1990) described two different categories of “sequence-related incomplete aminoacylations”. Random difficult aminoacylations were thought to occur where the peptide bond is formed between two residues with bulky side chains. Non-random difficult aminoacylations were described as characterised by reproducible repetitive incomplete aminoacylations, i.e. “difficult sequences”, which are thought to be associated with beta sheet formation. Milton et al. suggested the use of a solubility metric P*_{c} to predict reaction success for a chain, based on the Chou-Fasman parameters Pₐ and P_{b} of the residues in the chain (see Chou & Fasman, 1978). These are empirically derived parameters that characterise the probability for each amino acid to appear in an alpha helix or a beta sheet. However, this approach has not been able to reliably predict the occurrence of aggregation during synthesis, nor has it enabled elucidation of the factors affecting aggregation.

More recently, the development of flow-based peptide synthesis methods (also referred to as “automated fast-flow peptide synthesis”, AFPS) where each reaction can be monitored in real time, has enabled the development of more complex methods for predicting peptide synthesis outcome. In particular, Mohapatra et al. (2020) proposed a deep learning model trained using a data set containing 17,459 unique reaction steps characterised by the preceding amino acid sequence, the features of the incoming amino acid, and a set of synthesis parameters. Each of these is associated with UV-vis deprotection traces quantifying the time-dependent UV-vis signal associated with deprotection of fluorenylmethyloxycarbonyl (Fmoc) groups, which can be used to determine both synthesis efficiency and mass transfer issues during deprotection indicative of aggregation. The deep learning model was trained to predict the integral, height and width of UV-vis Fmoc deprotection traces for each coupling based on a topological representation of the pre-chain and incoming amino acids (extended-connectivity fingerprints, ECFP) and synthesis parameters (coupling agent, coupling strokes, deprotection strokes, flow rate).

While this approach showed some success in predicting aggregation, it is intrinsically linked to AFPS and requires large amounts of high-quality data obtained from this platform. Most industrial production of peptides is performed using SPPS in batch productions that does not produce this type of data. Thus, the present inventors have realised that there was an unmet need for methods that could predict whether a peptide can be successfully manufactured using such widely available chemical synthesis platforms. The inventors further identified that due to the extensive use of SPPS methods, there was untapped potential in the data that is associated with this use. This data is not suitable to train models such as those in Mohapatra et al. (2020) and Milton et al. (1990) and has, to the best of the inventors' knowledge, not been exploited for this purpose. Prior to the present invention, the potential use of this data had not been recognised, investigated or even postulated. Thus, the inventors set out to investigate whether they could develop a machine learning approach that uses data from batch SPPS to predict the outcome of peptide manufacture. They have surprisingly found that it was possible to obtain a machine learning model that can successfully predict the outcome of peptide chemical synthesis based solely on the peptide sequences, and without requiring data from individual couplings for the training of the model. That such a successful prediction was even possible using this approach was surprising, especially in light of previously unsuccessful attempts to characterise the sequence of “difficult peptides”.

Thus, the present inventors have developed a new method for predicting the outcome of peptide production by chemical synthesis that addresses one or more of the problems of prior art approaches. This method finds particular use in the production of diverse sets of peptides, for example for personalised therapy. The method uses a machine learning approach to predict metrics that characterise the outcome of the production of peptides by chemical synthesis. In its simplest form, the method is able to provide such predictions solely based on the primary structure of the peptides. Further, the machine learning approach uses models trained using data that is obtainable from batch chemical synthesis of peptides, which is the most commonly available type of peptide synthesis. Thus, the method can rapidly predict which peptides are likely to be difficult to make by chemical synthesis, compare related peptides for their likelihood of successful chemical synthesis, and select a subset of peptides from a candidate set that are more likely to be successfully synthesised. Further, the method can make use of large amounts of data available to peptide manufacturers through extensive use of existing chemical synthesis processes.

Thus, according to a first aspect, there is provided a method comprising: providing a primary amino acid sequence for one or more peptides, and predicting the outcome of production of the one or more peptides by chemical synthesis using a machine learning model that has been trained to predict one or more metrics characterising the outcome of production of peptides using training data comprising training peptide primary sequences and measured metrics characterising the outcome of production of the training peptides by chemical synthesis, wherein the measured metrics are metrics obtained after completion of the chemical synthesis process. Thus, the method may be a method of predicting the outcome of production of one or more peptides by chemical synthesis.

The method of the present aspect may have one or more of the following features.

The methods described herein may be computer implemented. At least the step of predicting the outcome of production may be computer implemented. The method may be a method for producing one or more peptides. The method may be a method for predicting the outcome of production of one or more peptides. The method may be a method for selecting peptides for chemical synthesis.

The machine learning model may take as input one or more features derived from the primary structure of peptides. The machine learning model may take as input the value of one or more process parameters. The machine learning model may take as input one or more features selected from a set of candidate features using a feature selection process. The feature selection process may comprise using a regularisation technique and/or removing features that show a correlation in the training data above a predetermined threshold. The one or more features may be standardised or may have been standardised prior to being used as input to the machine learning model. Standardisation may be performed using predetermined parameters, such as parameters derived from a training data set. The features derived from the primary structure of a peptide may be selected from: features quantifying the amino acid composition of the peptide, features indicative of the secondary structure of the peptide derivable from its primary structure, features indicative of the propensity of the peptide to aggregate during manufacture, features associated with the behaviour of the peptide in solution, features indicative of physico-chemical properties of the peptide, and features learned by a peptide sequence model. Features quantifying the amino acid composition of the peptide may comprise the percentage or proportion of each of one or more amino acids or groups of amino acids. Features indicative of the secondary structure of the peptide derivable from its primary structure may comprise the predicted proportion or percentage of amino acids in alpha helixes, beta chains and/or turns, and scores quantifying the propensity for amino acids in a chain to form alpha helixes, beta chains and/or turns. Scores quantifying the propensity for amino acids in a chain to form alpha helixes, beta chains and/or turns may comprise the Chou-Fasman parameters Pₐ, P_{b} and Pₜᵤᵣₙ. Features indicative of the propensity of the peptide to aggregate during manufacture may comprise scores that aggregate amino acid based metrics quantifying likelihood of aggregation. The amino acid based metrics may be empirical metrics. The features indicative of the propensity of the peptide to aggregate during manufacture may comprise the aggregation parameters P_{agg} and P*_{c}. Features associated with the behaviour of the peptide in solution may comprise metrics indicative of the solubility of the peptide, instability of the peptide, and flexibility of the peptide. A metric indicative of solubility may be a summarised solubility score calculated using amino acid specific solubility scores. A metric indicative of solubility may be the average of the amino acid specific solubility scores S provided in Table 2. A metric indicative of the instability of a peptide may be an instability metric calculated by summing empirical weight values associated with dipeptides in proteins and quantifying the impact of said dipeptides on protein stability, normalised by the length of the peptide. A metric indicative of the flexibility of a peptide may be a metric based on average flexibility parameters over windows of a fixed length. Features indicative of physico-chemical properties of the peptide may comprise the density, charge at a particular pH (e.g. charge at pH=7), isoelectric point, chromatographic retention time, a feature indicative of hydrophobicity, and molar extinction coefficient of the peptide. A feature indicative of hydrophobicity may be selected from: the aromaticity or the gravy score. Features learned by a peptide sequence model may comprise features learned by one or more neural network models trained to learn encodings of unlabelled peptide data.

The one or more peptides may have a length of at least 12, 13, 14, 15, 16, 17, 18, 19 or 20 amino acids. The one or more peptides may have a length of at least 15 amino acids. The one or more peptides may have a length of at most 50, 45, 40, 35, 34, 33, 32, 31 or 30 amino acids. The one or more peptides may have a length of at most 40 amino acids. The one or more peptides may have a length between 15 and 40 amino acids or between 20 and 35 amino acids. The one or more peptides may have a length within the same length boundaries as the peptides in the training data. Thus, the peptides in the training data may similarly have a length of at least 12, 13, 14, 15, 16, 17, 18, 19 or 20 amino acids, a length of at most 50, 45, 40, 35, 34, 33, 32, 31 or 30 amino acids, a length between 15 and 40 amino acids, and/or a length between 20 and 35 amino acids.

The chemical synthesis (for which the outcome is to be predicted and/or which was used to obtain the training data) may be a solid phase peptide synthesis. The chemical synthesis (for which the outcome is to be predicted and/or which was used to obtain the training data) may be a batch process. The chemical synthesis for which the outcome is predicted may use a similar process to the chemical synthesis process used to produce the training data. A similar process may be a process that uses the same instrument(s), instrument(s) of the same type, the same protection chemistry, the same activators, the same number of equivalents per coupling, the same concentrations of reagents, the same detection system, and/or the same additives.

Predicting the outcome of production of the one or more peptides by chemical synthesis may comprise predicting the value of the one or more metrics characterising the outcome of production of the peptide, wherein the metrics characterising the outcome of production of the peptide are metrics derivable from a chromatographic analysis of the composition resulting from the process of chemical synthesis of the peptide(s). The chromatographic analysis may be a LC or LC-MS analysis. Predicting the outcome of production of the one or more peptides by chemical synthesis may comprise predicting the value of the one or more metrics characterising the outcome of production of the peptide selected from: the purity of the resulting composition, one or more features of one or more chromatographic peaks associated with the composition, the identity of one or more products of the composition, whether the composition satisfies one or more criteria that apply to said metrics, the probability that the composition satisfies one or more criteria that apply to said metrics, and metrics derived from said metrics combining features for multiple chromatographic peaks. Predicting the outcome of production of the one or more peptides by chemical synthesis may comprise predicting the purity of the composition resulting from the process of chemical synthesis of the one or more peptides, and/or predicting whether the purity satisfies one or more criteria and/or predicting the probability that the purity satisfies one or more criteria. Purity of a composition may be defined for one or more target peptides in relation to a chromatogram of said composition as the percentage area of one or more chromatographic peak(s) in the chromatogram corresponding to the target peptides relative to the total area of the chromatogram. The one or more criteria may comprise a minimum purity.

The machine learning model may comprise a regression model or a classification model. The machine learning model may comprise a linear model. The machine learning model may comprise a neural network. The machine learning model may be a regularised model. A regularised model may be a L1 or L2 regularised linear regression or L1 or L2 regularised linear classification model. The machine learning model may comprise a regularised logistic regression model and/or regularised linear regression model.

The machine learning model may take as input the value of one or more process parameters, wherein process parameters are parameters that characterise how a particular chemical synthesis process is run. A process parameter may be a parameter set by a user or measured prior to, during or subsequent to carrying out the process. One or more process parameters may be selected from: the type, model or identifier of a synthesis instrument, the identity of an operator, the batch number of one or more reagents used to perform the synthesis, the value of one or more physico-chemical variables associated with the process, the value of one or more flows of solutions in the instrument, the presence or concentration of one or more activators, and the maintenance status of the instrument or any part thereof.

The machine learning model may take as input one or more features learned by a peptide sequence model, wherein the peptide sequence model comprises one or more neural network models trained to learn encodings of unlabelled peptide data. The one or more neural network models may be sequential models. The one or more neural network models may be deep neural networks. The one or more neural network models may be selected from: autoencoders and transformers. The one or more neural networks may be autoencoders. The one or more neural networks may be selected from recurrent neural networks, long short term memory networks, variational autoencoders, neural variational document models (NVDMs), or Wasserstein autoencoders. The one or more neural networks may have been trained in an unsupervised manner using training data comprising at least 1000 peptides, at least 2000, at least 3000, at least 4000, at least 5000 peptides, or at least 10,000 peptides. The one or more neural networks may have been trained as part of the supervised training of a neural network comprising the one or more neural networks generating encodings of unlabelled peptide data, the encodings being used as input to a neural network regressor or classifier trained to predict the one or more metrics characterising the outcome of production of peptides. The one or more neural networks may have been trained in an unsupervised manner using training data comprising peptide sequences drawn from a collection of peptides and/or proteins from a reference sequence, peptide sequences drawn from a previously obtained data set, randomly sampled peptides, or combinations thereof.

The machine learning model may take as input one or more features selected from the features listed in Table 3 or equivalents thereof. The machine learning model may take as input a plurality of features selected from the features listed in Table 3 or equivalents thereof. The machine learning model may take as input at least 5, at least 10 or at least 15 features listed in Table 3 or equivalents thereof. The machine learning model may take as input substantially all of the features listed in Table 3 or equivalents thereof. The machine learning model may be a linear regression or logistic regression model taking as input the features listed in Table 3 and equivalents thereof. The model may have the coefficients of any of the models listed in Table 3 or equivalent coefficients learned by fitting logistic regression or linear regression models to a particular training data set. Features that are equivalent to those listed in Table 3 may comprise features that quantify substantially the same property in a different manner, such as e.g. using different empirical formulae or different empirical parameters. The machine learning model may take as input one or more features quantifying the amino acid composition of the peptide. The features may include one or more of the percentage or proportion of Cys, Pro, Ser, Arg and Met. The features may include at least one of or all of the percentage or proportion of Cys, Pro and Arg.

The machine learning model may have been trained using cross-validation. The machine learning model may have been trained using a training data set comprising data for at least 1500, at least 2000, at least 3000, at least 4000, or at least 5000 peptides. The training data may comprise data for a plurality of peptide production batches and cross-validation was performed in a manner that did not split data in the same batch between cross-validation training and test sets.

The method may comprise predicting the outcome of production of a plurality of peptides by chemical synthesis. The method may comprise ranking or otherwise prioritising the plurality of peptides using one or more of the predicted metrics characterising the outcome of production of the peptides. The method may comprise providing to a user, for example through a user interface, one or more results of the method, optionally comprising the one or more predicted metrics characterising the outcome of the peptide production and/or a value derived therefrom or associated therewith and/or the sequence of one or more peptides selected from a plurality of peptides for which an outcome of production has been predicted. The method may comprise predicting the outcome of production by chemical synthesis of a plurality of candidate peptides, and selecting one or more of the candidate peptides that satisfy one or more predetermined criteria including at least one criterion that applies to a predicted metric characterising the outcome of producing the one or more candidate peptides. The method may comprise predicting the outcome of production by chemical synthesis of one or more candidate peptides and one or more peptides derived from the candidate peptides by shifting, trimming and/or substituting one or more positions in the sequence of the candidate peptides, and selecting one or more of the candidate peptides that satisfy one or more predetermined criteria including at least one criterion that applies to a predicted metric characterising the outcome of producing the one or more candidate peptides. The at least one criterion that applies to the predicted outcome of producing the one or more candidate peptides may be selected from: having a predicted purity that is above a predetermined threshold, having a predicted purity that is above a threshold set adaptively to select a predetermined number of candidate peptides with the highest predicted purity, and having a predicted purity that is above a threshold set adaptively to select a predetermined top percentile of candidate peptides. The predetermined number or top percentile of candidate peptides may also satisfy one or more further criteria. Selecting one or more of the candidate peptides may comprise selecting the one or more candidate peptides for synthesis with a first or second process. Selecting one or more of the candidate peptides may comprise selecting the one or more candidate peptide for synthesis using a process that includes more purification steps, different reagent concentration(s), different temperatures, or a different chemistry from a process to be used for one or more peptides that are not selected. The selected peptides may be neoantigen peptides comprising a tumour-specific mutation that satisfies at least a criterion selected from: being likely to be clonal, being associated with an expression product that is expressed in tumour cells, being predicted to result in a protein or peptide that is not expressed in the normal cells of the subject, being predicted to result in at least one peptide that is likely to be presented by an MHC molecule, preferably an MHC allele that is known to be present in the subject, and being predicted to result in a protein or peptide that is immunogenic.

According to a second aspect, there is provided a method of providing a tool for predicting the outcome of production of peptides by chemical synthesis, the method comprising: (i) obtaining a training data set comprising: training peptide primary sequences and measured metrics characterising the outcome of production of the training peptides by chemical synthesis, wherein the measured metrics are metrics obtained after completion of the chemical synthesis process; and (ii) providing a machine learning model that predicts the values of the one or more metrics characterising the output of chemical synthesis of peptides in the training data.

The method of the present aspect may have any one or more of the following features. The method according to the present aspect may have any of the features disclosed in relation to the first aspect. In particular, references to features of the trained model in relation to the first aspect may be interpreted as active steps of training the model in relation to the present aspect. The method of the present aspect may further have any one or any combination of the following optional features. Step (i) may further comprise obtaining the value(s) of one or more process parameters. Step (i) may further comprise obtaining the value of one or more features derived from the primary structure of peptides in the training data. The method may further comprise one or more of: providing the machine learning model to a user, data storage device or computing device, providing the image analysis algorithm to a user, data storage device or computing device. Obtaining a training data set may comprise synthesising a plurality of peptides and measuring the corresponding values of one or more metrics characterising the outcome of peptide synthesis process. Obtaining training data may comprise receiving data from a computer, computer readable medium or user interface.

As the skilled person understands, the complexity of the operations described herein (due at least to the complexity of obtaining predictive features and predictions as described herein, and the amount of data and computation that is typically required for training of machine learning models) are such that they are beyond the reach of a mental activity. Thus, unless context indicates otherwise (e.g. where sample preparation or acquisition steps are described), all steps of the methods described herein are computer implemented.

According to a third aspect, there is provided a method of producing one or more peptides, the method comprising: predicting the outcome of production of one or more candidate peptides the method of any embodiment of the first aspect, selecting one or more of the candidate peptides for chemical synthesis based on the results of the predicting, such as by selecting candidate peptides that satisfy one or more predetermined criteria including at least one criterion that applies to the predicted outcome of producing the one or more candidate peptides, and optionally producing the selected one or more selected peptides by chemical synthesis.

The method of the present aspect may have any one or more of the following features. The method may comprise predicting the outcome of production by chemical synthesis of one or more candidate peptides and one or more peptides derived from the candidate peptides by shifting, trimming and/or substituting one or more positions in the sequence of the candidate peptides, and selecting one or more of the candidate peptides and peptides derived from the candidate peptides that satisfy one or more predetermined criteria including at least one criterion that applies to a predicted metric characterising the outcome of producing the one or more peptides.

The at least one criterion that applies to the predicted outcome of producing the one or more peptides may be selected from: having a predicted purity above a predetermined threshold, having a predicted purity that is above a threshold set adaptively to select a predetermined number of candidate peptides with the highest predicted purity, and having a predicted purity that is above a threshold set adaptively to select a predetermined top percentile of candidate peptides. The predetermined number or top percentile of candidate peptides may also satisfy one or more further criteria.

Selecting one or more of the candidate peptides (and optionally peptides derived therefrom) may comprise selecting a first subset of the peptides for synthesis with a first process and a second subset of the peptides for synthesis with a second process. For example, the first process may include more purification steps, different reagent concentration(s), different temperatures, or a different chemistry, compared to the first process. The first and second subset may each be subsets in the strict sense, or empty or complete subsets.

The method may comprise synthesising the first subset using the first process. The method may comprise synthesising the second subset with the second process.

According to a fourth aspect, there is provided a method of monitoring a process for chemical synthesis of peptides. The method comprises: predicting the outcome of producing one or more peptides using the method of any embodiment of the first aspect, thereby obtaining predicted values of one or more metrics characterising the outcome of production of the one or more peptides; obtaining a measured value of one or more metrics characterising the outcome of production of the one or more peptides using the process; and comparing the measured values and the predicted values. Deviation between the measured and predicted value is indicative of a deviation between the expected and observed performance of the process. The obtaining may comprise synthesising the one or more peptides and determining the value of the one or more metrics. Instead or in addition to this, the obtaining may comprise providing previously measured values. A deviation between measured and predicted value may be a difference, a significant difference (e.g. subject to statistical testing), an increasing difference, and/or a difference above a predetermined threshold (e.g. in absolute value). A deviation may be determined for a plurality of peptides (also referred to as a “batch”). The predicted metrics may be compared to corresponding measured metrics. The predicted metrics for a respective peptide may comprise the probability that a composition resulting from synthesis of the respective peptide satisfies one or more criteria that apply to: the purity of the resulting composition, one or more features of one or more chromatographic peaks associated with the composition, and/or the identity of one or more products of the composition. The measured metrics for a respective peptide may comprise whether a composition resulting from synthesis of the respective peptide satisfies one or more criteria that apply to: the purity of the resulting composition, one or more features of one or more chromatographic peaks associated with the composition, and/or the identity of one or more products of the composition. The predicted metrics for a plurality of peptides may comprise a summarised metric (e.g. average, median) over the plurality of peptides, of the probability that a composition resulting from synthesis of each of the respective peptide satisfies one or more criteria that apply to: the purity of the resulting composition, one or more features of one or more chromatographic peaks associated with the composition, and/or the identity of one or more products of the composition. The measured metrics for a plurality of peptides may comprise the proportion of the plurality of peptides for which a composition resulting from synthesis of the respective peptide satisfies one or more criteria that apply to: the purity of the resulting composition, one or more features of one or more chromatographic peaks associated with the composition, and/or the identity of one or more products of the composition. The method may further comprise, based on the deviation, determining that the process performance is lower than expected (e.g. deviation with a first sign), that the process performance is higher than expected (e.g. deviation with a second sign), that the process performance is degrading over time (e.g. deviation with a first sign and that increases in absolute value when repeating the method for a plurality of peptides synthesised using the process at a plurality of different times), or that the process is improving over time (e.g. deviation with a second sign and that increases in absolute value when repeating the method for a plurality of peptides synthesised using the process at a plurality of different times). The method may further comprise identifying a set of one or more peptides that have been synthesised using the process with at least one common implementation parameter and for which the deviation is indicative of a process performance that is lower than expected. A common implementation parameter may be selected from: the same one or more machines, the same operator, the same synthesis batch, the same batch of one or more reagents, The same value of one or more operating parameters. The method may further comprise modifying the common implementation parameter prior to synthesis of one or more further peptides.

According to a fifth aspect, there is provided a method of providing an immunotherapy for a subject that has been diagnosed as having cancer, the method comprising: optionally identifying one or more cancer neoantigens for the subject or obtaining a set of one or more candidate neoantigens for the subject, wherein the one or more candidate neoantigens were identified using a process comprising analysing one or more samples from the subject comprising tumour genetic material, and designing an immunotherapy that targets one or more of the cancer neoantigens, wherein the designing comprises predicting the outcome of production of one or more peptides identified for at least one of the candidate neoantigens using the method of any of embodiment of the first aspect. The one or more neoantigens may be clonal neoantigens. The immunotherapy that targets the one or more of the neoantigens may be an immunogenic composition, a composition comprising immune cells or a therapeutic antibody. The method may further comprise producing one or more peptides selected from the identified peptides and/or producing an immunotherapy using one or more peptides selected from the identified peptides. The immunotherapy that targets the one or more of the cancer neoantigens may be an immunogenic composition, a composition comprising immune cells or a therapeutic antibody. Also provided are: methods for expanding a T cell population for use in the treatment of cancer in a subject, compositions comprising a population of T cells obtained or obtainable by such a method, compositions comprising a neoantigen peptide, neoantigen peptide specific immune cell, or an antibody that recognises a neoantigen peptide, for use in the treatment or prevention of cancer in a subject, wherein said neoantigen peptide has been identified using the methods described herein, compositions comprising a neoantigen peptide, neoantigen peptide specific immune cell, or an antibody that recognises a neoantigen peptide, wherein said neoantigen peptide has been produced using the methods described herein, neoantigen peptide, immune cell which recognises a neoantigen peptide, or antibody which recognises a neoantigen peptide, for use in the treatment or prevention of cancer in a subject, wherein said neoantigen peptide has been produced using the methods described herein, uses of said products and compositions in the manufacture of a medicament for use in the treatment or prevention of cancer in a subject, and methods of treating a subject that has been diagnosed as having cancer, the method comprising administering an immunotherapy that has been provided using the methods described herein, or a composition as described herein.

According to a further aspect, there is provided a system comprising: a processor; and a computer readable medium comprising instructions that, when executed by the processor, cause the processor to perform the steps of any method described herein, such as a method according to any embodiment of the first, second, third, fourth or fifth aspects above. The system may further comprise peptide synthesising means. The system may further comprise one or more sensors for measuring one or more parameters of the peptide synthesis process performed by the peptide synthesising means and/or one or more peptide analysis means. The peptide analysis means may comprise a high-pressure liquid chromatography instrument and/or a mass spectrometry instrument. Preferably, the peptide analysis means comprises both a high-pressure liquid chromatography instrument and a mass spectrometry instrument (also referred to as a combined LC-MS instrument).

According to a further aspect, there is provided or more computer readable media comprising instructions that, when executed by one or more processors, cause the one or more processors to perform the steps of any method described herein, such as a method according to any embodiment of the first, second, third, fourth or fifth aspects above.

According to a further aspect, there is provided a computer program comprising code which, when the code is executed on a computer, causes the computer to perform the steps of any method described herein, such as a method according to any embodiment of the first, second, third, fourth or fifth aspects above.

### BRIEF DESCRIPTION OF THE FIGURES

FIG. 1 is a flowchart illustrating schematically a method of predicting the outcome of production of peptides and a method of providing a tool according to the disclosure.

FIG. 2 is a flowchart illustrating schematically a method of providing an immunotherapy for a subject.

FIG. 3 shows an embodiment of a system for producing peptides.

FIG. 4 shows example LC-MS data for peptides with high (A) and low (B) purity. Each plot shows a chromatographic trace (top) and mass spectrometry spectrum for a selected window of chromatographic retention time (bottom) The tables below the plots show the chromatographic peaks automatically quantified.

FIG. 5 shows the results of investigation of the purity variability (A) and pass rate variability (B) by batch for data sets of peptides manufactured by two different manufacturers. A. These plots show the standard deviation of passed peptides purity in each of the plurality of batches analysed. The continuous line represents the standard deviation of purity over all batches and the dotted line represents variability of +/−two standard deviations in this number. B. Pass rate by manufacturer is recorded. The solid line represents the average value of this number over all observations and the dotted line represents +/−two standard deviations in this number.

FIG. 6 shows the results of analysis of repeat batches performed by the same manufacturer (A) and by two different manufacturers (B). On the left, a scatter plot uses one run to explain the other. On the right, a confusion matrix using one run to predict pass/fail in the other.

FIG. 7A shows the results of an analysis of correlation between expert defined sequence derived features of peptides in a data set of manufactured peptides. The heatmap shows Pearson correlation coefficients between features. FIG. 7B shows the weights for the predictive features in trained models for pass/fail prediction for two manufacturers (MFG1, MFG2) for pass/fail classification models (balanced logistic regression and logistic regression, respectively top and second row for each manufacturer) and purity prediction regression models. The weights for the logistic regression model and balanced regression models were very similar.

FIG. 8 shows the performance of Ridge and Logistic Regression models predicting purity and pass/fail for two different manufacturers (top and bottom rows). Left plots are predicted vs realised purity. Middle plots show a histogram of regression residuals with zero purity observations in orange. Right plot is a confusion matrix. The zero purity peptides cause the residual distribution to be non-normal. Excluding these, the distributions look normal.

FIG. 9 shows the feature importance as L1 regularisation in the models of FIG. 8 is increased. A. Regression model, MFG1. B. Classification model, MFG1. C. Regression model, MFG2. D. Classification model, MFG2.

FIG. 10 shows regression and classification errors by batch (MFG1). Top: left bars show purity standard deviations by batch. Right bars are regression residuals by batch—the unexplained variance. Bottom: variation in false positive and negative rates by batch is studied alongside pass rate variability.

FIG. 11 shows learning curves for Ridge (A) and Logistic Regression (B) models by batch, using MFG1 data.

FIG. 12A shows a diagram of sequential VAE and WAE models (VLSTM and WLSTM) used in the examples. The encoder learns a latent state z that is used at each step by the decoder. V or W LSTMs are configured by changing the distance loss (top right).

FIG. 12B shows a diagram of NVDM model used in the examples.

FIG. 13 shows the training loss with KL annealing for the NVDM model used in the examples. Turning on the KL divergence later in training allows successful annealing from the deterministic to the regularised state.

FIG. 14 shows the cross validated performance of a L2 regularised logistic regression using expert defined features and learned features from various unsupervised models.

FIG. 15 shows fully random (left), randomly sampled from the proteome (middle), and synthesised neoantigen (right) peptides in the space of the WLSTM encoder used in the examples, projected into 2-d.

FIG. 16 shows a diagram of a supervised neural prediction framework used in the examples. This framework accepts non-linear interactions, via adding layers and non-linear activations in the output network. Feature models like the WLSTM can be added to generate features from sequence data in addition to expert defined features. Feature models operating on sequence data can also be learned at training time, if they are allowed to have gradients backpropagated through them.

FIG. 17 shows the prediction performance of neural models as described on FIG. 17 for MFG 1 data.

FIG. 18 shows the activations of LSTM models in a classification task demonstrated in the examples.

FIG. 19 shows learning curves for the LSTM model of FIG. 18 using MFG 1 data.

FIG. 20 shows the simulated effect of tuning on pass rate (A) and purity (B) of batches.

FIG. 21 shows the results of an investigation of the reliability of model calibration for models of the disclosure, for two different manufacturers (A: MFG1, B: MFG2). Classification models (logistic regression model for prediction of “pass” probability) were calibrated using manufacturer-specific training data and tested using a holdout test dataset from the same manufacturer. The predictions were binned by pass probability then (i) the average predicted pass probability per bin was compared with the corresponding average observed pass rate for the bin (top graph, continuous line shows the identity line), and (ii) the number of observed passing vs failed peptides were plotted for each predicted probability bin (bottom graph, left bar in each group shows the numbers of failed peptides predicted as passing peptides and the right bar shows the number of passing peptides predicted as passing peptides).

FIG. 22 demonstrates the use of models described herein to monitor process performance over time. X axis is time in months, Y axis is difference between predicted and observed pass rate (proportion of peptides in a batch predicted as “pass” vs. actually manufactured to required “pass” criteria) of any particular batch. Continuous line shows the 60-day average. Dots show data for individual batches, coloured by location relative to the 95% confidence interval (proportion of pass peptides for the batch below, within or above the 95% confidence interval for a difference between two binomial proportions).

FIG. 23 shows for a plurality of batches (x-axis), the range of predicted pass probabilities for the peptides in the batch (small dot and whiskers showing the median and interquartile range) using models as described herein, together with the observed pass rate (proportion of peptides in the batch that were manufactured to the required “pass” criteria), ordered by increasing observed pass rate. A. MFG1. B. MFG2.

### DETAILED DESCRIPTION

In describing the present invention, the following terms will be employed, and are intended to be defined as indicated below.

The disclosure relates at least in part to the prediction of metrics characterising or indicative of the outcome of chemical synthesis of one or more peptides.

The term “peptide” is used in the normal sense to mean a series of residues, typically L-amino acids, connected one to the other typically by peptide bonds between the a-amino and carboxyl groups of adjacent amino acids. The term includes modified peptides and synthetic peptide analogues. In particular, peptides as used herein may include one or more non-canonical amino acids (also referred to as “nonstandard amino acids” or “modified amino acids”. Further, in the context of providing features derived from the primary structure of a peptide comprising one or more non-canonical amino acids, as will be described further below, the features may be assumed to have the same values as that for a corresponding non-modified peptide, a predetermined value or a value derived therefrom (e.g. a value for the peptide derived using a default value for the noncanonical amino acid), or a value specific to the peptide or noncanonical amino acid (such as e.g. when the specific values are available or can be learned for the respective noncanonical amino acid). The methods described herein are particularly useful for the prediction of the outcome of chemical synthesis of peptides that are long enough to be non-trivial to synthesise (such as e.g. at least 12, 13, 14, 15, 16, 17, 18, 19 or 20 amino acids, preferably at least 15 amino acids) and short enough to be likely possible to synthesise chemically using a SPPS batch method and/or to be satisfactorily modelled using features associated with the primary sequence or predicted secondary structure of the peptides (such as e.g. at most 50, 45, 40, 35, 34, 33, 32, 31 or 30 amino acids, preferably at most 40 amino acids). Thus, reference to “peptides” herein may refer to amino acids chains of between 12 and 50 residues, such as between any of 12, 13, 14, 15, 16, 17, 18, 19 or 20 residues and any of 50, 45, 40, 35, 34, 33, 32, 31 or 30 residues, for example between 15 and 40 residues, between 15 and 35 residues, between 20 and 35 residues or between 20 and 30 residues. The present methods may be particularly useful in the context of production of peptides that are difficult to make by chemical synthesis, such as e.g. sets of peptides that comprise tumour neoantigens, or sets of peptides that are on average more hydrophobic than random peptides. Peptides may be characterised by one or more features derived from the peptide's primary structure. In the context of the present disclosure, one or more features derived from the peptide's primary structure are used as predictive features to predict one or more metrics characterising or indicative of the outcome of chemical synthesis of the peptide. The primary structure (also referred to simply as “sequence”) of a peptide refers to the linear sequence of amino acids that form the peptide. By convention, primary structures are provided from the amino-terminal (N) end of the peptide to the carboxyl-terminal (C) end of the peptide. Features derived from the primary structure of a peptide may include one or more of:
features quantifying the amino acid composition of the peptide sequence (such as e.g. the percentage or proportion of each of one or more amino acids or groups of amino acids),
features indicative of the secondary structure of the peptide derivable from primary structure (such as e.g. the predicted proportion or percentage of amino acids in alpha helixes, beta chains and/or turns, where prediction of the secondary structure adopted by a peptide sequence can be obtained using one or more algorithms for secondary and/or tertiary structure as known in the art; scores quantifying the propensity for amino acids in a chain to form alpha helixes, beta chains and/or turns),
features indicative of the propensity of peptides to aggregate during manufacture, such as scores that aggregate amino acid based metrics (such as e.g. empirical metrics) quantifying likelihood of aggregation,
features associated with the behaviour of the peptide in solution, such as metrics indicative of the solubility of the peptide, instability of the peptide, and flexibility of the peptide, and
features indicative of physico-chemical properties of the peptide such as density, charge at a particular pH (e.g. charge at pH=7), isoelectric point, chromatographic retention time, hydrophobicity, and molar extinction coefficient.

All of the above features may be referred to as “expert defined features”. They are typically defined by an expert based on knowledge of peptides physicochemical properties and/or peptide synthesis. Features derived from the primary structure of a peptide may also include features learned by peptide sequence models, typically deep learning models trained to learn features relevant to the “language” of proteins in an unsupervised manner or in a supervised manner as part of a model (e.g. a neural model) trained to predict the outcome of peptide manufacturability as described herein. Such features are referred to herein as “learned features”. In embodiments, one or more neural network models (also referred to as deep learning models or deep neural networks) are used to learn features derived from peptides by training the models to learn a latent representation of peptide sequences from which the peptide sequences can be reconstructed by the model. One or more latent variables comprised in the latent representation of peptide sequences learned by the neural network model(s) can then be used as predictive features to predict the outcome of peptide synthesis as described herein. For example, one or more variables of the latent state from a model comprising an encoder and a decoder may be used as predictive features. The one or more neural network models may be any models developed for or suitable for learning encodings of unlabelled data, for example for the purpose of natural language processing or computer vision. The one or more neural network models may have been trained or may be trained as part of a method described herein using peptide data (such as e.g. peptide sequences) drawn from a collection of peptides and/or proteins from a reference sequence (e.g. peptides drawn from the proteome sequence of a relevant organism), from a previously obtained data set (such as e.g. previously manufactured peptides, proteomic data sets, etc.), randomly sampled peptides (such as e.g. peptide sequences drawn sequentially at random using predetermined distributions of amino acid representations, where the predetermined distributions may capture the likelihoods of each of a set of amino acids being sampled—for example, each amino acid may be individually drawn by sampling a uniform distribution and including an amino acid depending on the bin in the [0,1] interval in which the value falls, wherein the bins may be defined to give equal or different likelihoods of sampling to each of a set of amino acids), or combinations thereof. In embodiments, the one or more models are trained using sequences drawn from a reference sequence. For example, the models may be trained using sequences drawn from the human proteome. The peptides used for training of these neural network models may have lengths within the same boundaries as those used to train the models for predicting the output of production as described herein. The one or more models may have been trained or may be trained as part of the methods described herein using training data comprising at least 1000 peptides, at least 2000, at least 3000, at least 4000, at least 5000 peptides, or at least 10,000 peptides. Preferably, the one or more models is/are sequential models. Sequential models are designed to analyse sequences of data, where the points in a sequence are not independent from each other. The models may be sequence-to-sequence models. The models may comprise an encoder and a decoder, where the latent state from the encoder is used as an input to the decoder for each prediction of an element of the output sequence. In embodiments, the one or more models is/are selected from: autoencoders (preferably recurrent neural networks such as long short term memory networks (LSTM), variational autoencoders (also referred to as variational LSTM), neural variational document models (NVDMs), Wasserstein autoencoders (WAE, also referred to as WLSTM)) and transformers. In embodiments, the one or more models are regularised autoencoders. Regularised autoencoders may be trained using one or more regularisation terms (such as e.g. KL divergence or MMD distance as explained further in the examples below). Regularisation terms may reduce the risk of overfitting the training data. For example, KL divergence and MMD distance penalise autoencoder networks if they learn a posterior distribution different to N(0, I). Other regularisation approaches as known in the art may be used.

A particular example of a feature quantifying the amino acid composition of a peptide sequence is the percentage or proportion of an individual of the 20 canonical amino acids listed in Table 2. When the percentage or proportion of each of these amino acids are used as features, this leads to a total of 20 individual features. One or more non-canonical amino acids may also be included instead or in addition to canonical amino acids. In embodiments, the features derived from the primary structure of a peptide includes at least a plurality of features quantifying the amino acid composition of a peptide sequence, such as the percentage or proportion of a plurality of individual amino acids. In embodiments, instead or in addition to these, the features derived from the primary structure of a peptide include one or more learned features. The learned features may be latent variables from a sequential model. The use of learned features may be particularly advantageous when large amounts of training data are available, such as e.g. training data comprising data for at least 5,000 or at least 10,000 peptides. As will be explained further below, specific features may be found to be more or less informative depending on a particular predictive model, such that some features may be excluded entirely from a model. Examples of algorithms for secondary and/or tertiary structure prediction include IntFOLD (McGuffin et al., 2015), ESyPred3D (Lambert et al., 2002), ROBETTA (Baek et al., 2021), SWISS-MODEL (Waterhouse, 2018), HHpred (Söding, 2005), PSIPRED (Buchan and Jones, 2019), etc. Examples of scores quantifying the propensity for amino acids in a chain to form alpha helixes, beta chains and/or turns include the Chou-Fasman parameters Pₐ, P_{b} and Pₜᵤᵣₙ provided in Table 2 (and these may be summarised for a peptide using any summary statistic known in the art such as e.g. a median or average, based on these parameters or metrics derived therefrom), the secondary structure fraction defined as the fraction of amino acids which tend to be in helix, turn or sheet (where amino acids which tend to be in helix may be defined as: V, I, Y, F, W, L; amino acids which tend to be in turns may be defined as: N, P, G, S; and amino acids which tend to be beta sheets: E, M, A, L). Examples of scores that aggregate amino acid based metrics quantifying likelihood of aggregation include the aggregation parameter P_{agg} calculated as the average amino acid specific P_{agg} over the chain where amino acid specific P_{agg} are empirical metrics quantifying the propensity of residues to be present in chains that aggregate as measure by a swellometer (as provided e.g. in Table 2), and P*_{c} (Milton et al., 1990—calculated as the average over the chain of P, =1/(0.739 Pₐ+0.345 P_{b}) where Pₐ, P_{b} are the Chou-Fasman parameters for each amino acid in the chain, such as provided in Table 2). The hydrophobicity of a peptide may be predicted using one or more metrics such as aromaticity (which quantifies the relative frequency of aromatic amino acids in a sequence) or the gravy score (GRand AVerage of hydropathicity (gravy) according to Kyte and Doolittle, 1982, which is the average of amino acid specific hydropathy scores such as provided in Table 2). Examples of metrics indicative of the solubility of a peptide include a summarised solubility score calculated based on amino acid specific solubility scores, for example the average of the amino acid specific solubility scores S in Table 2. Examples of metrics indicative of the instability of a peptide include the instability metric defined in Guruprasad et al. (1990), which sums empirical weight values associated with dipeptides in proteins and quantifying the impact of said dipeptides on protein stability, normalised by the length of the peptide. A particular example of a metric indicative of the flexibility of a peptide is the metric described in Vihinen et al (1994) which computes average flexibility parameters over windows of a fixed length (9 residues was found by Vihinen et al. to be optimal in their particular set up).

The terms “chemical synthesis”, “chemical peptide synthesis”, “peptide synthesis” or simply “synthesis” are used interchangeably to refer to any chemical method of synthesis of peptides or proteins, as opposed to synthesis through recombinant technologies. In the context of the present disclosure, the synthesis is typically solid-phase synthesis (also known as solid-phase peptide synthesis, SPPS), where peptides are synthesised on a solid support by successive deprotection and coupling of each subsequent amino acid. Peptides synthesised by SPPS are typically up to about 80 amino acids in length. Longer peptides can be obtained by ligation of peptides of lengths achievable by SPPS. For example, two deprotected peptides synthesised by SPPS may be ligated in solution (also referred to as “native chemical ligation”). Multiple types of SPPS exist depending primarily on the choice of protecting groups used during peptide synthesis. All such types are envisaged herein, including Fmoc protected synthesis (using a fluorenylmethoxycarbonyl protecting group), Boc-protected synthesis (using a terbutoxycarbonyl protecting group) and Smoc protected synthesis (using a disulfo-fluorenylmethoxycarbonyl). Typically, an Fmoc protected synthesis is used. The methods described herein may have improved performance when training data using the same type of chemistry is used as the process for which an outcome of production is to be predicted, compared to predicting the outcome of production for a process that is different from that sued to obtain the training data. In this context, the use of the methods and tools described herein for prediction of outcome of production using Fmoc protected synthesis may be particularly advantageous as such a process is common and training data more likely to be readily available. Nevertheless, other chemistries could be used, particularly if training data is available for these. Preferably, the SPPS is a batch method, not a flow method. In a batch method, each successive coupling is performed in a closed reaction vessel, and the content of the reaction vessel is heated and/or exchanged at discrete steps. Reaction vessels of a few ml or dozens of ml to multiple litres can be used depending on the scale of the production process. For example, reaction vessels of 20 ml to multiple litres (up to 1001 or more) may be used. The methods disclosed herein are particularly useful when making diverse sets of peptides on relatively small scales, for example custom sets of peptides (also referred to as “panels”) for a particular application (e.g. treatment of a particular subject). Thus, reaction vessels of between 20 ml and 51, or 20 ml and 11 may be used. By contrast, in a continuous flow method (also referred to as “flow”, “rapid-flow” or “micro-flow” peptide synthesis), a stream of reagent is passed through a relatively low volume reaction vessel. Typically, the stream of reagent is passed through a heat exchanger prior to entering the reaction vessel, and exits said reaction vessel through a UV detector which enables continuous monitoring of the process. Flow-based peptide synthesis processes are described e.g. in Simon et al., (2014).

The methods described herein are applicable to peptides obtained through batch or flow-based SPPS. However, without wishing to be bound by theory, the inventors believe that the methods described herein are particularly advantageous in the context of batch SPPS as very limited information about individual couplings can be obtained using such methods. This is in contrast with flow based SPPS where the efficiency of individual couplings can be estimated and used to train machine learning models as described in Mohapatra et al. (2020).

The terms “outcome”, “metrics characterising the outcome” and “metrics indicative of the outcome” refer to the characteristics of the composition resulting from a process of chemical synthesis of one or more peptides. These characteristics may be derived from a chromatographic elution profile associated with the composition resulting from the process of chemical synthesis of the peptide(s). These may include one or more characteristics selected from: the purity of the resulting composition, one or more features (such as e.g. height, width, area, % area, number of peaks) of one or more chromatographic peaks associated with the composition, the identity of one or more products of the composition (as assessed based on the size of the products determined using e.g. mass spectrometry and/or HPLC), and whether the composition satisfies one or more criteria that apply to the above metrics, as well as metrics derived from the above such as metrics combining chromatographic features for multiple peaks (e.g. peaks corresponding to the full length peptide to be produced, peaks corresponding to peptides within a predetermined distance of said peptide, peaks corresponding to impurities). The purity of the composition may be defined in relation to one or more target peptides for each peptide to be produced. The term “target peptides” refers to all peptides that are a desired product of the synthesis. These may include the full-length peptide, peptides within a predetermined distance of the full length peptide, and/or peptides with predetermined sequence features. The term “impurities” may refer to peptides that are not the full-length peptide, peptides that are not within a predetermined distance of the full length peptide, and/or peptides that have or do not have predetermined sequence features. Purity may be defined in relation to target peptides and/or in relation to impurities. For example, the purity of the composition may be defined in relation to the target peptides that have the full length of the peptide to be produced. Alternatively, the purity of the composition may be defined based on a plurality of target peptides within a predetermined length of the full-length peptide to be produced. Purity may be defined as the ratio of the size of the chromatographic peak(s) for the one or more target peptides relative to the size of all other chromatographic peaks (also referred to as % area for one or more peaks). Alternatively, purity may be defined as the ratio of the size of the chromatographic peak(s) for the one or more target peptides relative to the size of one or more chromatographic peaks for one or more impurities. In the examples provided below, the term “purity” is used to refer to the default meaning of the % area of the chromatographic peak associated with the full-length peptide to be synthesised. The identity of the peptides associated with one or more chromatographic peaks can be assessed by mass spectrometry as known in the art. One or more criteria that apply to the above metrics may be selected from: a criterion that applies to the identity of the peptides in the dominant peak (peak with largest % area) in the chromatogram (where identity can be determined by mass spectrometry), a criterion that applies to the characteristics of the peak(s) in the chromatogram that correspond to the full length peptide to be manufactured, a criterion that applies to the characteristics of the peak(s) in the chromatogram that correspond to non-full length peptides within a predetermined distance of the full length peptide, a criterion that applies to the characteristics of peaks that do not correspond to the full length peptide to be manufactured (and optionally also do not correspond to non-full length peptides within a predetermined distance of the full length peptide). An example of a criterion that applies to the identity of the peptides in the dominant peak in the chromatogram includes whether the dominant peak corresponds to peptide(s) that are within a target list, where the target list may include the full length target peptides and optionally one or more peptides that are within a certain distance from the full length target peptide (e.g. no more than 1, 2, 3, 4, 5, 6, 7, 8, 9 or 10 amino acids shorter). An example of a criterion that applies to the characteristics of the peak(s) in the chromatogram that correspond to the full length peptide to be manufactured includes whether said peak(s) are dominant, whether said peaks have a % area above a predefined threshold (e.g. 5, 10, 15, 20, 25, 30, 35, 40, 45, 50, 55, or 60%), whether said peaks are within a predetermined distance from one or more other peaks (such as e.g. peaks corresponding to impurities that are preferably removed). Similar criteria can be applied to peaks that correspond to non-full-length peptides within a predetermined distance of the full length peptide, for example when these peptides are also desirable products. An example of a criterion that applies to the characteristics of peaks that do not correspond to the full-length peptide (or other peptides that are within a predetermined distance) to be manufactured includes the number and/or size of such peaks.

The term “process parameter” refers to a parameter that characterises how a particular synthesis process is run. A process parameter can be set by a user or measured prior to, during or subsequent to carrying out the process. Process parameters may include one or more parameters selected from: the particular instrument used to perform the synthesis, the identity of an operator, the batch number of one or more reagents used to perform the synthesis, the value of one or more physico-chemical variables associated with the process (e.g. temperature, pH, concentration(s) of one or more reagents), the value of one or more flows (in flows or outflows) of solutions in the instrument (such as e.g. the amount of solvent flowing in or out of the process, the amount of waste product exiting the instrument during a synthesis), the maintenance status of the instrument or any part thereof (such as e.g. the length of time elapsed since a filter was replaced), the presence or concentration of one or more activators, and any other experimental parameter that may influence the synthesis process. The machine learning models described herein used to predict one or more metrics characterising the output of a chemical peptide synthesis may take as inputs the values of one or more process parameters. The specific identity of the one or more process parameters may depend on the context and in particular on the particular chemical synthesis process (e.g. type of instrument used) as well as the metrics to be predicted. Further, whether a candidate process parameter influences the outcome of a chemical synthesis may be determined by including the candidate process parameter as a further input of a machine learning model trained/fitted to predict the one or more metrics characterising the output of the chemical synthesis, and determining whether the resulting trained/fitted model is predictive of the one or more metrics of interest. The features of the resulting trained/fitted model may further be investigated (such as e.g. the coefficients of a linear model, the weights or a regression tree, etc.) in order to determine whether the candidate parameter contributes significantly to the prediction made by the model. Instead or in addition to this, a feature selection process as known in the art may be applied during the training/fitting of the statistical model to identify variables that are predictive of the metrics of interest.

According to the present disclosure, the outcome of the chemical synthesis of one or more peptides is predicted using one or more machine learning models. A “machine learning model” may also be referred to herein as a “statistical model” or “mathematical model”. The term “machine learning model” refers to a mathematical model that has been trained to predict one or more output values based on input data, where training refers to the process of learning, using training data, the parameters of the mathematical model that result in a model that can predict outputs values that satisfy an optimality criterion or criteria. In the case of supervised learning, training typically refers to the process of learning, using training data, the parameters of the mathematical model that result in a model that can predict outputs values that with minimal error compared to comparative (known) values associated with the training data (where these comparative values are commonly referred to as “labels”). The term “machine learning algorithm” or “machine learning method” refers to an algorithm or method that trains and/or deploys a machine learning model. Regression models can be seen as machine learning models. Conversely, some machine learning models can be seen as regression models in that they capture the relationship between a dependent variable (the values that are being predicted) and a set of independent variables (the values that are used as input to the machine learning model, from which the machine learning model makes a prediction). Any machine learning regression model may be used according to the present disclosure as a statistical model to predict metrics characterising the output of a peptide chemical synthesis. A model that predicts one or more metrics characterising the output of a peptide chemical synthesis may be trained by using a learning algorithm to identify a function F: p→mᵢ where F is a function parameterised by a set of parameters θ such that:

$${m}_{i} \approx {\hat{m}}_{i} = F ( p \vert θ ) \qquad ( 1 )$$

where \hat{m}ᵢ is a predicted metric characterizing the output of a peptide chemical synthesis, and ρ is a set of predictor variables including one or more features derived from the primary structure of a peptide for which the metric(s) m are to be predicted, and an optional set of additional predictor variables such as process parameters, and θ is a set of parameters identified as satisfying equation (2):

$$θ = arg {min}_{θ} L ( {m}_{i} , {\hat{m}}_{i} ) \qquad ( 2 )$$

where L is a loss function that quantifies the model prediction error based on the observed and predicted metrics characterizing the output of a peptide chemical synthesis. The specific choice of the function F, parameters θ and function L as well as the specific algorithm used to find 0 (learning algorithm) depends on the specific machine learning method used. Any method that satisfies the equations above can be used within the context of the present disclosure, including in particular any choice of loss function, model type and architecture. In embodiments, a statistical model (also referred to herein as “machine learning model”) that may be used to predict one or more metrics characterizing the output of a peptide chemical synthesis is a linear regression model. In embodiments, a statistical model that may be used to predict one or more metrics characterizing the output of a peptide chemical synthesis is a non-linear regression model. A linear regression model is a model of the form according to equation (3), which can also be written according to equation (3b):

$$Y = X β + ε \qquad ( 3 )$$

$$\begin{matrix}{y}_{i} = {β}_{0} + {β}_{1} {x}_{i 1} + … {β}_{p} {x}_{ip} + {ε}_{i} & i = 1 , … , n\end{matrix} \qquad ( 3 b )$$

where Y is a vector with n elements yᵢ (one for each dependent/predicted variable), X is a matrix with elements xᵢ₁ . . . xᵢₚ for each of the p predictor variables and each of the n dependent variables, and n elements of 1 for the intercept value, β is a vector of p+1 parameters, and E is a vector of n error terms (one for each of the dependent variables). Linear regression models can be seen as neural networks consisting of a single artificial neuron, or a single layer neural network. Thus, the machine learning model used to predict the output of chemical synthesis may be a neural network that comprises an artificial neuron or single layer neural network for linear regression. The model may comprise additional elements for example for providing input predictive features, which may themselves be neural networks as described above and further in the examples. In embodiments, the machine learning model used to predict one or more metrics characterizing the output of a peptide chemical synthesis is a truncated regression model, such as a truncated linear or non-linear regression model. A truncated regression model may be useful when part of the training data to be modeled is less reliable or best excluded, such as e.g. peptides associated with purity below a threshold. In embodiments, the machine learning model used to predict one or more metrics characterizing the output of a peptide chemical synthesis is a linear classification model. For example, the model may be or may comprise a linear discriminant analysis model, a naïve Bayes classifier, a logistic regression model (including a neural network representation of a logistic regression model), a perceptron, or a support vector machine. In embodiments, a machine learning model that may be used to predict one or more metrics characterizing the output of a peptide chemical synthesis is a non-linear classification model. For example, the model may be or may comprise a k-nearest neighbor classifier, a kernel SVM, a decision tree (including a random forest), or a multilayer perceptron. In embodiments, the model is chosen from any machine learning model known in the art suitable for performing a binary classification. In embodiments, the model is a logistic regression model. A logistic regression model, also referred to simply as “logistic model”, is a model that learns a sigmoid function (also referred to as logistic function) to predict a dependent variable (typically a binary dependent variable) from one or more predictive variables. A logistic regression model is a model of the form according to equation (4), which can also be written according to equation (4b):

$$p = ( P ( Y = 1 ) ) = \frac{1}{1 + {b}^{- ( {β}_{0} + {β}_{1} {x}_{1} + … {β}_{p} {x}_{p} + ε )}} \qquad ( 4 )$$

$$p = {S}_{b} ( {β}_{0} + {β}_{1} {x}_{1} + … {β}_{p} {x}_{p} + ε ) \qquad ( 4 b )$$

Where S_{b} is the sigmoid function in base b, x₁ . . . xₚ are predictor variables and p is the probability that response variable Y takes the value 1. Preferably, the machine learning model (whether a classification model, a regression model or otherwise) used to predict the output of chemical synthesis is a linear model.

In embodiments, a machine learning model is an artificial neural network (ANN, also referred to simply as “neural network” (NN)). ANNs are typically parameterized by a set of weights that are applied to the inputs of each of a plurality of connected neurons in order to obtain a weighted sum that is fed to an activation function to produce the neuron's output. The parameters of an NN can be trained using a method called backpropagation through which connection weights are adjusted to compensate for errors found in the learning process, in combination with a weight updating procedure such as stochastic gradient descent. An ANN may be a deep neural network, i.e. a neural network comprising more than one layer (also referred to as “hidden layer”) between the input layer and the output layer. An ANN may be a convolutional neural network (CNN, or ensemble of CNNs). In embodiments, a machine learning model comprises an ensemble of models whose predictions are combined. Alternatively, a machine learning model may comprise a single model. The machine learning model may comprise a plurality of individual machine learning models. Theses may have been trained to perform the same task (in which case they machine learning model may be referred to as an “ensemble model”. Alternatively, the machine learning model may comprise a plurality of machine learning models (each of which may comprise a single model or an ensemble model) trained in a supervised manner to predict one or more metrics characterizing the output of a peptide chemical synthesis, wherein the one or more signals differ between the plurality of machine learning models. In embodiments, a machine learning model may be trained to predict a single metric characterizing the output of a chemical peptide synthesis. Alternatively, a machine learning model may be trained to jointly predict a plurality of metrics characterizing the output of a chemical peptide synthesis. In such cases, the loss function used may be modified to be an (optionally weighted) average across all variables that are predicted, as described in equation (5):

$${L}_{K} ( m , \hat{m} ) = \frac{1}{n} {\sum }_{i ∈ k} {α}_{i} L ( {m}_{i} , {\hat{m}}_{i} ) \qquad ( 5 )$$

where αᵢ are optional weights that may be individually selected for each of the k metrics i, m and \hat{m} are the vectors of actual and predicted metrics. Optionally, the values of mᵢ may be scaled prior to inclusion in the loss function (e.g. by normalizing so that the labels for all the jointly predicted variables have equal variance), for example to reduce the risk of some of the jointly predicted \hat{m}ᵢ dominating the training. A linear regression model may be selected from a simple linear regression model, a multiple linear regression model, a partial least square regression model, an orthogonal partial least square regression, a random forest regression model, a decision tree regression model, a support vector regression model, and a k-nearest neighbour regression model. The statistical model may have been obtained by training a statistical model to predict the one or more metrics characterizing the output of chemical peptide synthesis using training data comprising the primary structure of peptides previously synthesised and the corresponding values of the one or more metrics characterizing the output of chemical peptide synthesis for these peptides. The training data may comprise, for a plurality of peptides: the primary structure of the peptides, or features derived from said primary structure; the value(s) of one or more further predictive features, such as e.g. process parameters; and corresponding values of the one or more metrics characterizing the output of chemical peptide synthesis. The wording “corresponding values” means that the values are obtained for chemical synthesis performed to obtain the one or more peptides, and to which the further predictive features relate. In other words, the statistical model is trained/fitted to predict metrics of interest for a peptide synthesis based on predictive variables for the same peptide synthesis. Preferably, the training data comprises or consists of data that relates to chemical synthesis performed using the same type of chemical synthesis as that for which an outcome is to be predicted. Preferably, the training data comprises or consist of data that relates to chemical synthesis performed using the same peptide synthesis means (e.g. same type and/or model of instrument) as that for which an outcome is to be predicted. The training data may comprise data for at least 1500 peptides, at least 2000, 3000, 4000, or 5000 peptides. The training data may be divided between a training set and a test set. The training set may comprise data for at least 1500 peptides, at least 2000, 3000, 4000, or 5000 peptides. The test set may comprise data for at least 100, at least 200, 300, 400, 500, 600, 700 or 800 peptides. The training of the model may be performed using cross-validation. The training of the model may be performed including or excluding one or more peptides in the training data that do not meet one or more criteria applying to metrics indicating the outcome of peptide manufacturing. For example, models trained to predict a metric indicative of the outcome of manufacturing which is a continuous metric such as e.g. purity may be trained using training data that includes only peptides for which purity was measured (as opposed to e.g. imputed).

In embodiments, the machine learning model (whether a classification model, a regression model or otherwise) used to predict the output of chemical synthesis is a regularized model. Regularized models may reduce the risk of overfitting models to training data. The machine learning model used to predict the output of chemical synthesis may be L1 regularized (also referred to as “Lasso regression” in the case of a L1 regularized regression or logistic regression model) or L2 regularized (also referred to as “Ridge regression” in the case of a L2 regularized regression or logistic regression model). L2 regularized models include a squared penalty term in the loss function, i.e. a term that includes the squared value(s) of the model coefficient(s). L1 regularized models include an absolute value penalty term in the loss function, i.e. a term that includes the absolute value(s) of the model coefficient(s). Regularization may shrink the coefficient of less important features to zero, thus resulting in feature selection. The level of regularization (whether L1 or L2) can be tuned using a parameter (typically referred to as A), that weights the penalty term in the loss function. Increasing the weight of the penalty term disincentivizes the model from giving any input variable too much importance unless its predictive value is high, thus providing an indication of the importance of the input variables by studying their coefficients in the model as regularization penalty is increased. In embodiments, the machine learning model (whether a classification model, a regression model or otherwise) used to predict the output of chemical synthesis is trained using cross validation and/or feature selection. Any feature selection process known in the art may be used, including but not limited to L1 regularization. In embodiment, the machine learning model may have been trained using features selected from a set of candidate features by removing candidate features that have a correlation above a predetermined threshold (such as e.g. above 80%, above 90%, above 95%) in the training data or any other suitable data set. Preferably, the machine learning model (whether a classification model, a regression model or otherwise) used to predict the output of chemical synthesis is trained using cross validation. In embodiments, the predictive variables are normalized, preferably standardized (i.e. z-scored) prior to input in the model.

A “sample” as used herein may be a cell or tissue sample, a biological fluid, an extract (e.g. a DNA extract obtained from the subject), from which genomic material can be obtained for genomic analysis, such as genomic sequencing (e.g. whole genome sequencing, whole exome sequencing). The sample may be a cell, tissue or biological fluid sample obtained from a subject (e.g. a biopsy). Such samples may be referred to as “subject samples”. In particular, the sample may be a blood sample, or a tumour sample, or a sample derived therefrom. The sample may be one which has been freshly obtained from a subject or may be one which has been processed and/or stored prior to genomic analysis (e.g. frozen, fixed or subjected to one or more purification, enrichment or extraction steps). The sample may be a cell or tissue culture sample. As such, a sample as described herein may refer to any type of sample comprising cells or genomic material derived therefrom, whether from a biological sample obtained from a subject, or from a sample obtained from e.g. a cell line. In embodiments, the sample is a sample obtained from a subject, such as a human subject. The sample is preferably from a mammalian (such as e.g. a mammalian cell sample or a sample from a mammalian subject, such as a cat, dog, horse, donkey, sheep, pig, goat, cow, mouse, rat, rabbit or guinea pig), preferably from a human (such as e.g. a human cell sample or a sample from a human subject). Further, the sample may be transported and/or stored, and collection may take place at a location remote from the genomic sequence data acquisition (e.g. sequencing) location, and/or any computer-implemented method steps described herein may take place at a location remote from the sample collection location and/or remote from the genomic data acquisition (e.g. sequencing) location (e.g. the computer-implemented method steps may be performed by means of a networked computer, such as by means of a “cloud” provider).

A “normal sample” or “germline sample” refers to a sample that is assumed not to comprise tumour cells or genetic material derived from tumour cells. A germline sample may be a blood sample, a tissue sample, or a purified sample such as a sample of peripheral blood mononuclear cells from a subject. Similarly, the terms “normal”, “germline” or “wild type” when referring to sequences or genotypes refer to the sequence/genotype of cells other than tumour cells. A germline sample may comprise a small proportion of tumour cells or genetic material derived therefrom, and may nevertheless be assumed, for practical purposes, not to comprise said cells or genetic material. In other words, all cells or genetic material may be assumed to be normal and/or sequence data that is not compatible with the assumption may be ignored.

The terms “tumour-specific mutation”, “somatic mutation” or simply “mutation” are used interchangeably and refer to a difference in a nucleotide sequence (e.g. DNA or RNA) in a tumour cell compared to a healthy cell from the same subject. The difference in the nucleotide sequence can result in the expression of a protein which is not expressed by a healthy cell from the same subject. For example, a mutation may be a single nucleotide variant (SNV), multiple nucleotide variant (MNV), a deletion mutation, an insertion mutation, a translocation, a missense mutation, a translocation, a fusion, a splice site mutation, or any other change in the genetic material of a tumour cell. A mutation may result in the expression of a protein or peptide that is not present in a healthy cell from the same subject. Mutations may be identified by exome sequencing, RNA-sequencing, whole genome sequencing and/or targeted gene panel sequencing and or routine Sanger sequencing of single genes, followed by sequence alignment and comparing the DNA and/or RNA sequence from a tumour sample to DNA and/or RNA from a reference sample or reference sequence (e.g. the germline DNA and/or RNA sequence, or a reference sequence from a database). Suitable methods are known in the art.

An “indel mutation” refers to an insertion and/or deletion of bases in a nucleotide sequence (e.g. DNA or RNA) of an organism. Typically, the indel mutation occurs in the DNA, preferably the genomic DNA, of an organism. In embodiments, the indel may be from 1 to 100 bases, for example 1 to 90, 1 to 50, 1 to 25 or 1 to 10 bases. An indel mutation may be a frameshift indel mutation. A frameshift indel mutation is a change in the reading frame of the nucleotide sequence caused by an insertion or deletion of one or more nucleotides. Such frameshift indel mutations may generate a novel open-reading frame which is typically highly distinct from the polypeptide encoded by the non-mutated DNA/RNA in a corresponding healthy cell in the subject.

A “neoantigen” (or “neo-antigen”) is an antigen that arises as a consequence of a mutation within a cancer cell. Thus, a neoantigen is not expressed (or expressed at a significantly lower level) by normal (i.e. non-tumour) cells. A neoantigen may be processed to generate distinct peptides which can be recognised by T cells when presented in the context of MHC molecules. As described herein, neoantigens may be used as the basis for cancer immunotherapies. References herein to “neoantigens” are intended to include also peptides derived from neoantigens. The term “neoantigen” as used herein is intended to encompass any part of a neoantigen that is immunogenic. An “antigenic” molecule as referred to herein is a molecule which itself, or a part thereof, is capable of stimulating an immune response, when presented to the immune system or immune cells in an appropriate manner. The binding of a neoantigen to a particular MHC molecule (encoded by a particular HLA allele) may be predicted using methods which are known in the art. Examples of methods for predicting MHC binding include those described by Lundegaard et al., O'Donnel et al., and Bullik-Sullivan et al. For example, MHC binding of neoantigens may be predicted using the netMHC-3 (Lundegaard et al.) and netMHCpan4 (Jurtz et al.) algorithms. A neoantigen that has been predicted to bind to a particular MHC molecule is thereby predicted to be presented by said MHC molecule on the cell surface.

A “clonal neoantigen” (also sometimes referred to as “truncal neoantigen”) is a neoantigen that results from a mutation that is present in essentially every tumour cell in one or more samples from a subject (or that can be assumed to be present in essentially every tumour cell from which the tumour genetic material in the sample(s) is derived). Similarly, a “clonal mutation” (sometimes referred to as “truncal mutation”) is a mutation that is present in essentially every tumour cell in one or more samples from a subject (or that can be assumed to be present in essentially every tumour cell from which the tumour genetic material in the sample(s) is derived). Thus, a clonal mutation may be a mutation that is present in every tumour cell in one or more samples from a subject. A “sub-clonal” neoantigen is a neoantigen that results from a mutation that is present in a subset or a proportion of cells in one or more tumour samples from a subject (or that can be assumed to be present in a subset of the tumour cells from which the tumour genetic material in the sample(s) is derived). Similarly, a “sub-clonal” mutation is a mutation that is present in a subset or a proportion of cells in one or more tumour samples from a subject (or that can be assumed to be present in a subset of the tumour cells from which the tumour genetic material in the sample(s) is derived). As the skilled person understands, a neoantigen or mutation may be clonal in the context of one or more samples from a subject while not being truly clonal in the context of the entirety of the population of tumour cells that may be present in a subject (e.g. including all regions of a primary tumour and metastasis). Thus, a clonal mutation may be “truly clonal” in the sense that it is a mutation that is present in essentially every tumour cell (i.e. in all tumour cells) in the subject. This is because the one or more samples may not be representative of each and every subset of cells present in the subject. Thus, within the context of the present disclosure, a “clonal neoantigen” or “clonal mutation” may also be referred to as a “ubiquitous neoantigen” or “ubiquitous mutation”, to indicate that the neoantigen is present in essentially all tumour cells that have been analysed, but may not be present in all tumour cells that may exist in the subject. The terms “clonal” and “ubiquitous” are used interchangeably unless context indicates that reference to “true clonality” was intended. The wording “essentially every tumour cell” in relation to one or more samples or a subject may refer to at least 70%, at least 75%, at least 80%, at least 85%, at least 90%, at least 91%, at least 92%, at least 93%, at least 94% at least 95%, at least 96%, at least 97%, at least 98%, or at least 99% of the tumour cells in the one or more samples or the subject.

A cancer immunotherapy (or simply “immunotherapy”) refers to a therapeutic approach comprising administration of an immunogenic composition (e.g. a vaccine), a composition comprising immune cells, or an immunoactive drug, such as e.g. a therapeutic antibody, to a subject. The term “immunotherapy” may also refer to the therapeutic compositions themselves. In the context of the present disclosure, the immunotherapy typically targets a neoantigen. For example, an immunogenic composition or vaccine may comprise a neoantigen, neoantigen presenting cell or material necessary for the expression of the neoantigen. As another example, a composition comprising immune cells may comprise T and/or B cells that recognise a neoantigen. The immune cells may be isolated from tumours or other tissues (including but not limited to lymph node, blood or ascites), expanded ex vivo or in vitro and re-administered to a subject (a process referred to as “adoptive cell therapy”). Instead or in addition to this, T cells can be isolated from a subject and engineered to target a neoantigen (e.g. by insertion of a chimeric antigen receptor that binds to the neoantigen) and re-administered to the subject. As another example, a therapeutic antibody may be an antibody which recognises a neoantigen. One skilled in the art will appreciate that if the neoantigen is a cell surface antigen, an antibody as referred to herein will recognise the neoantigen. Where the neoantigen is an intracellular antigen, the antibody will recognise the neoantigen peptide-MHC complex. As referred to herein, an antibody which “recognises” a neoantigen encompasses both of these possibilities. Further, an immunotherapy may target a plurality of neoantigens. For example, an immunogenic composition may comprise a plurality of neoantigens, cells presenting a plurality of neoantigens or the material necessary for the expression of the plurality of neoantigens. As another example, a composition may comprise immune cells that recognise a plurality of neoantigens. Similarly, a composition may comprise a plurality of immune cells that recognise the same neoantigen. As another example, a composition may comprise a plurality of therapeutic antibodies that recognise a plurality of neoantigens. Similarly, a composition may comprise a plurality of therapeutic antibodies that recognise the same neoantigen.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

References to “an immune cell” are intended to encompass cells of the immune system, for example T cells, NK cells, NKT cells, B cells and dendritic cells. In a preferred embodiment, the immune cell is a T cell. An immune cell that recognises a neoantigen may be an engineered T cell. A neoantigen specific T cell may express a chimeric antigen receptor (CAR) or a T cell receptor (TCR) which specifically binds a neoantigen or a neoantigen peptide, or an affinity-enhanced T cell receptor (TCR) which specifically binds a neoantigen or a neoantigen peptide (as discussed further hereinbelow). For example, the T cell may express a chimeric antigen receptor (CAR) or a T cell receptor (TCR) which specifically binds to a neo-antigen or a neo-antigen peptide (for example an affinity enhanced T cell receptor (TCR) which specifically binds to a neo-antigen or a neo-antigen peptide). Alternatively, a population of immune cells that recognise a neoantigen may be a population of T cell isolated from a subject with a tumour. For example, the T cell population may be generated from T cells in a sample isolated from the subject, such as e.g. a tumour sample, a peripheral blood sample or a sample from other tissues of the subject. The T cell population may be generated from a sample from the tumour in which the neoantigen is identified. In other words, the T cell population may be isolated from a sample derived from the tumour of a patient to be treated, where the neoantigen was also identified from a sample from said tumour. The T cell population may comprise tumour infiltrating lymphocytes (TIL).

The term “Antibody” (Ab) includes monoclonal antibodies, polyclonal antibodies, multispecific antibodies (e.g., bispecific antibodies), and antibody fragments that exhibit the desired biological activity. The term “immunoglobulin” (Ig) may be used interchangeably with “antibody”. Once a suitable neoantigen has been identified, for example by a method according to the disclosure, methods known in the art can be used to generate an antibody.

An “immunogenic composition” is a composition that is capable of inducing an immune response in a subject. The term is used interchangeably with the term “vaccine”. The immunogenic composition or vaccine described herein may lead to generation of an immune response in the subject. An “immune response” which may be generated may be humoral and/or cell-mediated immunity, for example the stimulation of antibody production, or the stimulation of cytotoxic or killer cells, which may recognise and destroy (or otherwise eliminate) cells expressing antigens corresponding to the antigens in the vaccine on their surface. The immunogenic composition may comprise one or more neoantigens, or the material necessary for the expression of one or more neoantigens. In addition, a neoantigen may be delivered in the form of a cell, such as an antigen presenting cell, for example a dendritic cell. The antigen presenting cell such as a dendritic cell may be pulsed or loaded with the neo-antigen or neo-antigen peptide or genetically modified (via DNA or RNA transfer) to express one, two or more neoantigens or neoantigen peptides, for example 2, 3, 4, 5, 6, 7, 8, 9 or 10 neo-antigens or neoantigen peptides. Methods of preparing dendritic cell immunogenic compositions or vaccines are known in the art.

A “neoantigen peptide” as described herein refers to a peptide that comprises a cancer cell specific mutation (e.g a non-silent amino acid substitution encoded by a single nucleotide variant (SNV), an indel or any other genetic alteration that results in a change in primary structure of a peptide or protein) at any residue position within the peptide. By way of example, a peptide which is capable of binding to an MHC class I molecule is typically 7 to 13 amino acids in length. As such, the amino acid substitution may be present at position 1, 2, 3, 4, 5, 6, 7, 8, 9, 10, 11, 12 or 13 in a peptide comprising thirteen amino acids. In embodiments, longer peptides, for example 21-31-mers, may be used, and the mutation may be at any position, for example at the centre of the peptide, e.g. at positions 10, 11, 12, 13, 14, 15 or 16. Such peptides can also be used to stimulate both CD4 and CD8 cells to recognise neoantigens.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” or “computer device” includes the hardware, software and data storage devices for embodying a system or carrying out a method according to the above-described embodiments. For example, a computer system may comprise one or more processing units such as a central processing unit (CPU) and/or a graphical processing unit (GPU), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. For example, a computer system may be implemented as a cloud computer. The term “computer readable media” includes, without limitation, any non-transitory medium or media which can be read and accessed directly by a computer or computer system. A computer readable medium may be a tangible computer readable medium. A computer readable medium may be realized as a plurality of discrete tangible computer readable media. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Prediction of Outcome of Peptide Production

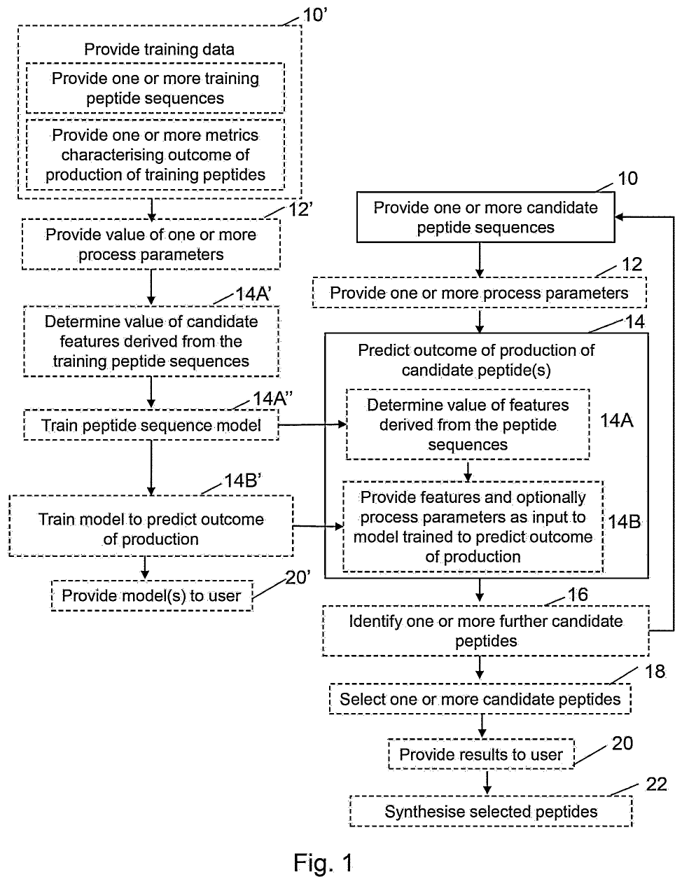

The present disclosure provides methods for predicting the outcome of production of one or more peptides, and methods for providing a tool for predicting the outcome of production of one or more peptides. An illustrative method for providing a tool and/or predicting the outcome of production of one or more peptides will be described by reference to FIG. 1. At step 10, sequence data for one or more candidate peptides is obtained. At optional step 12, the value of one or more process parameters is provided. These may be determined based on a process with which the peptides are planned to be synthesised. At step 14, the outcome of production of the candidate peptide sequences is predicted based on the candidate peptide sequences themselves and optionally also the process parameters. This may comprise predicting the value of one or more metrics characterising the outcome of production of the one or more candidate peptides, where these metrics are metrics that are obtainable after completion of the chemical synthesis process. In other words, these metrics characterise the product of a completed chemical synthesis of a peptide. This may comprise optional step 14A of determining the value of one or more features derived from the candidate peptide sequences. For example, one or more features derived from the candidate peptide sequences may be calculated at this point using predetermined methods for each of the respective features. For example, when the features comprise the percentage or proportion of each of a set of amino acids, these percentages or proportions may be calculated for each candidate sequence at step 14A. As another example, when the features comprise learned features from a deep neural network trained to determine encodings associated with peptide sequences, the encodings may be calculated at step 14A. At optional step 14B, the value of the one or more features and optional process parameters are provided as input to a machine learning model trained to predict the outcome of production of the peptides. Steps 14A and 14B may be combined for example using a machine learning model trained to determine the values of features derived from the peptide sequences and predict the outcome of production based on these. At optional step 16, one or more further candidate peptides may be identified. These may for example be based on the previously provided candidate peptides, such as e.g. peptides obtained by shifting, trimming or mutating one or more of the previously candidate peptides. Alternatively, these may be provided by a user, selected from a set from which the previously provided candidate peptides were selected or identified using the same method used to identify the previously provided candidate peptide sequences (such as e.g. by identifying further peptides comprising one or more mutations, by identifying one or more further peptides forming part of a scanning library, etc.). Any one or more of steps 10 to 14 may then be repeated for the one or more further candidate peptides. At step 18, one or more of the candidate peptides may be selected using one or more criteria including at least one criterion that applies to the predictions from step 14. For example, peptides with a predicted purity above a predetermined threshold may be selected. This may be combined with one or more further criteria that do not apply to the predictions from step 14. For example, the peptides may be peptides that each comprise one of a set of mutations, and the one or more peptides with highest predicted purity (such as e.g. the top 1, 2, 3, 4 or 5 peptides) may be selected for each set of peptides comprising a respective mutation from the set of mutations. At optional step 20, the results of one or more of steps 14, 16 and 18 may be provided to a user, for example through a user interface. At optional step 22, one or more of the peptides selected at step 18 may be synthesised. For example, the results of step 18 may be automatically provided to a peptide synthesising means or computer system associated therewith. The models used at steps 14A and 14B may have been previously trained or may be trained as part of a method described herein. Thus, also described is a method of providing a tool for predicting the outcome of production of one or more peptides by chemical synthesis. The method may comprise at step 10′, obtaining a training data set comprising: training peptide primary sequences and measured metrics characterising the outcome of production of the training peptides by chemical synthesis. The measured metrics are metrics obtained after completion of the chemical synthesis process. Step 10′ may comprise providing one or more training peptide sequences, synthesising the one or more training peptide sequences, and measuring the value of one or more metrics associated with the completed chemical synthesis process, such as metrics characterising the composition provided as final output of the chemical synthesis process. Alternatively, the training sequences and corresponding metrics may be obtained from a user, computing device or data store. At optional step 12′, the value of one or more process parameters associated with the training data may be provided. This may comprise the value of one or more process parameters used during the chemical synthesis process from which the training data was obtained. Instead or in addition to this, this may comprise one or more default values. At steps 14A′ to 14B′, a machine learning model that predicts the values of the one or more metrics characterising the output of chemical synthesis of peptides in the training data may be provided. This may comprise step 14A′ of determining the value of one or more candidate features derived from the training peptide sequences. The candidate features may be selected from a predetermined list, and may be obtained according to predetermined processes. This may instead or in addition comprise step 14A″ of training a peptide sequence model. The peptide sequence model comprises one or more neural network models trained to learn encodings of unlabelled peptide data. The training of the peptide sequence model may be performed in an unsupervised manner, by providing unlabelled training data comprising peptides sequences that may include some or all of the training peptide sequences or may be defined independently of the training peptide sequences, such as e.g. by sampling a reference proteome. The term “unlabelled” refers to training data that does not include the values to be predicted (i.e. the values characterising the outcome of production of the training peptides). Thus, unlabelled training data may consist of the peptide sequences. The training of the peptide sequence model may be performed in a semi-supervised manner, by using a pretrained model (e.g. a model pre-trained on unlabelled peptide sequence data), and at least partially retraining the peptide sequence model as part of the supervised training of a neural network comprising the one or more neural networks generating encodings of unlabelled peptide data, the encodings being used as input to a neural network regressor or classifier trained to predict the one or more metrics characterising the outcome of production of peptides. Steps 14A′ and/or 14A″ may further comprise the step of standardising the one or more candidate features. Providing a machine learning model that predicts the values of the one or more metrics characterising the output of chemical synthesis of peptides in the training data may further comprise step 14B′ of training a machine learning model to predicts the values of the one or more metrics characterising the output of chemical synthesis of peptides, using as input the value of one or more of the features determined at step 14A′ or provided as output of the sequence model(s) trained at step 14A″. Training the machine learning model may comprise selecting one or more features from the candidate features defined at step 14A′ and/or 14A″, for example using a feature selection process such as e.g. filtering and/or regularisation. Training the machine learning model may comprise identifying the value of one or more parameters of the model(s) that optimise a chosen criterion such as e.g. prediction loss. The method may further comprise step 20′ of providing the one or more models to a user. The model provided at steps 14A″ and/or 14B′ may comprise a single model or a plurality of models. These tools and methods described may be used for example to produce one or more peptides, for example for use in providing an immunotherapy, as will be described further below.

### Applications

The above methods find applications in the context of producing one or more peptides, preferably custom peptides for a specific application, such as for treatment of a subject, and/or in the context of producing a large variety of peptides, such as when providing a peptide library (examples of which include libraries prepared for Alanine-scans, D-amino acid scans or other similar scans of one or more sequences of interest), where experimental optimisation of production of each individual peptide is impractical. The methods described herein may also find use in analysing, defining or optimising a peptide production process, by obtaining a tool for predicting the outcome of peptide synthesis using a method as described herein and identifying one or more process parameters that are predictive of the outcome of peptide production, using a machine learning model as described herein. This may be performed for example by using the machine learning model to predict the outcome of production of a set of peptides when the machine learning model takes as input the values of one or more candidate process parameters, and identifying those candidate process parameters that significantly influence the predictions. Instead or in addition to this, one or more parameters of the model may be analysed to determine the predictive character of one or more process parameters. For example, when the machine learning model comprises a regression model, the coefficients of the regression model may be analysed to identify process parameters that significantly influence the predictions made by the model. Such methods may further comprise identifying the values of one or more of said process parameters that are predicted to result in an improved outcome of peptide production, by analysing the parameters of the machine learning model and/or by using the machine learning model to predict the outcome of production of a set of peptides using candidate values of said one or more process parameters. The methods herein find use in monitoring a process for chemical synthesis of peptides, by comparing observed and predicted metrics characterising the outcome of production of one or more peptides using the process. The monitoring may be performed for a particular batch of peptides, for example to ensure that each batch is produced with an expected level of performance (i.e. identify batches that may have been subject to one or more faults, errors, etc.). The monitoring may be performed for a plurality of batches of peptides, for example to ensure consistent performance over time or identify systematic faults, errors, etc.

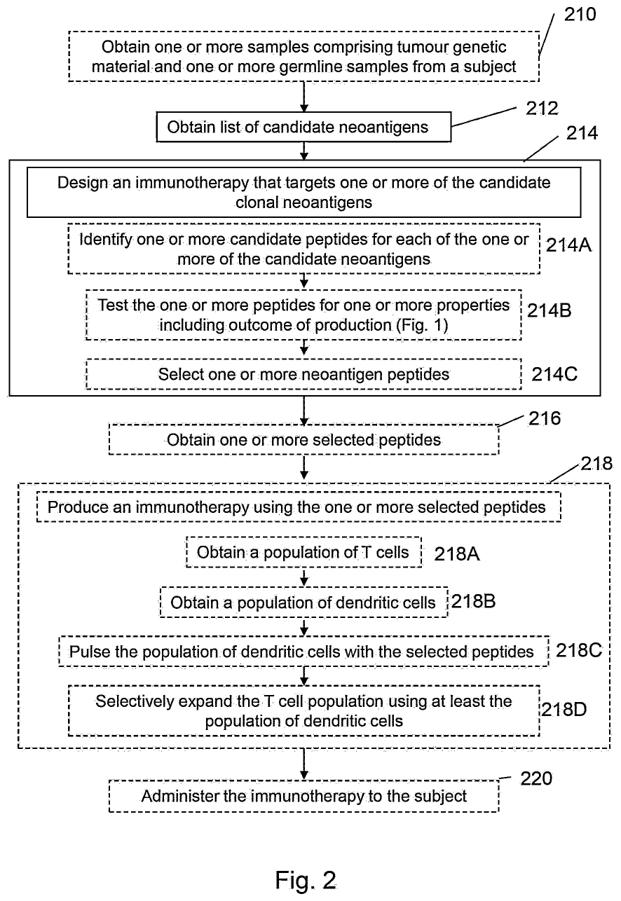

The methods described herein may be particularly useful in the context of providing or designing scanning libraries (where single residues in one or more sequences are replaced e.g. by an alanine or d-amino acid unit in order to e.g. identify a biologically active residue). As another example, the methods described herein are particularly useful in the context of personalised therapies, where the production of the therapy requires production of one or more peptides that are specific to the subject to be treated. For example, the above methods find applications in the context of immunotherapeutic approaches. In particular, the above methods may be used to provide cancer immunotherapies that target cancer-specific antigens (also referred to herein as “cancer neoantigens”, or simply “neoantigens”). As the skilled person understands, a cancer-specific antigen may be truly specific to cancer cells (in the sense that it is only expressed by the genome of cancer cells), or may be practically specific to cancer cells (in the sense that it is expressed by cancer cells at a significantly higher level than by normal cells). The cancer neoantigens may be clonal neoantigens. Thus, also described herein are methods of providing an immunotherapy for a subject, the method comprising identifying and optionally producing one or more peptides that comprise cancer neoantigens, wherein the identifying is based on data from one or more samples from the subject and further comprises predicting the output of manufacturing of the one or more peptides using a method as describe herein. An example of such a method will be described by reference to FIG. 2.

FIG. 2 illustrates schematically an exemplary method of providing an immunotherapy. At optional step 210, one or more samples comprising tumour genetic material and one or more germline samples are obtained from a subject. The subject may be a subject that has been diagnosed as having cancer, and may be (but does not need to be) the same subject for which the immunotherapy is provided. At step 212, a list of candidate neoantigens is obtained using methods known in the art, for example as described in WO 2016/16174085, Landau et al. (2013), Lu et al. (2018), Leko et al. (2019), Hundal et al. (2019), and others. The neoantigens may be clonal neoantigens. Methods to identify clonal neoantigens are known in the art and include the methods described in WO 2016/16174085, Landau et al. (2013), Roth et al. (2014), McGranahan et al. (2016), and in WO 2022/207925 The list may comprise a single neoantigen, or a plurality of neoantigens. Preferably, the list comprises a plurality of neoantigens. At step 214, an immunotherapy that targets at least one (and optionally a plurality) of the candidate neoantigens is designed. Designing such an immunotherapy comprises identifying one or more candidate peptides for each of the candidate clonal neoantigens (step 214A). For example, a plurality of peptides may be designed for at least one of the candidate clonal neoantigens, which differ in their lengths and/or the location of a sequence variation that characterises the neoantigen compared to the corresponding germline peptide. At step 214B, the one or more peptides identified are tested to evaluate the likely outcome of production of the peptide using a method as described herein, and optionally one or more additional properties such as their immunogenicity, likelihood of being displayed by a MHC molecule, etc. At step 214C, one or more of the peptides are selected based on at least some of the results of step 214B. The peptides may be selected for manufacture and/or processing using a particular process. For example, peptides may be selected for manufacture with a particular process for which the predicted outcome is better than for another candidate process. As another example, peptides may be selected for manufacture with an adapted process (e.g. using higher concentrations of reagents) if their predicted outcome satisfies one or more criteria indicating that the peptides are likely to be difficult to manufacture (e.g. predicted purity below a certain threshold). As another example, peptides may be selected for manufacture in combination with one or more downstream purification steps. One or more purification steps may be selected from: desalting, salt exchange, catch-and-release purification, prep LC, or any other peptide purification process used in the art. As another example, peptides may be selected for modification (e.g. by shifting or modifying the sequence), optionally followed by repeating step 214C. The peptides may be selected using one or more rounds of testing 214B and selection 216C, whereby peptides with an improved likely outcome of production are designed and selected. For example, a set of candidate peptides may be tested, then new candidate peptides may be generated corresponding to one or more of the candidate peptides tested (such as e.g. those that do not satisfy one or more criteria applying to the predicted outcome of synthesis) by shifting and/or trimming and/or otherwise modifying (such as e.g. by inclusion of substitute amino acids) the candidate peptides. These may be tested 214B again, then a set of peptides which may comprise peptides from the original set as well as peptides corresponding to these obtained through one or more rounds of testing and selection may be selected 216C.

At step 216, the selected peptides may be obtained. Peptides with selected sequences may be obtained using any method known in the art but they are preferably obtained using chemical synthesis. Preferably, the peptides are synthesised at step 216 using the chemical synthesis method for which the method of predicting the outcome of peptide production has been trained. At step 218, an immunotherapy may be produced using at least some of the one or more peptides produced at step 216. The immunotherapy may comprise the one or more peptides (e.g. in the case of an immunogenic composition such as a synthetic long peptide vaccine), or may comprise molecules or cells that have been obtained using the selected peptides (e.g. in the case of therapeutic antibodies that selectively bind the candidate peptides, or immune cells that specifically recognise the candidate peptides). In the illustrated embodiment, the immunotherapy comprises cells that have been obtained using the selected peptides. Methods of producing an immunotherapy comprising cells that have been obtained using neoantigen peptides are known in the art, for example as described in WO 2016/16174085, McGranahan et al. (2016), Lu et al. (2018), Leko et al. (2019), Robbins et al. (2013), and in co-pending application GR 20210100409. At optional step 220, the immunotherapy may be administered to a subject, which is preferably the subject from which the samples used to identify the cancer neoantigens have been obtained. An example of producing an immunotherapy comprising a T cell population selectively enriched with T cells that recognise one or more neoantigens, preferably clonal neoantigens, will be described. At step 218A, a population of T cells may be obtained. The T cells may be obtained from the subject to be treated, but do not need to be. The T cells may be obtained from a tumour sample, from a blood sample, or from any other tissue sample. At step 218B, a population of dendritic cells may be obtained. For example, a population of dendritic cells may be derived from mononuclear cells (e.g. peripheral blood mononuclear cells, PBMCs) from the subject to be treated. At step 218C, the population of dendritic cells may be pulsed with the selected peptides. At step 218D, the T cell population may be selectively expanded using the population of pulsed dendritic cells. Additional expansion factors such as e.g. cytokines or stimulating antibodies may be used.

Thus, the disclosure provides a method of providing an immunotherapy for a subject that has been diagnosed as having cancer, the method comprising: optionally identifying one or more cancer neoantigens for the subject, and designing an immunotherapy that targets one or more of the cancer neoantigens, wherein the designing comprises performing the method of the first aspect for one or more candidate peptides comprising the one or more of the cancer neoantigens. The method may have any one or more of the following features. The immunotherapy that targets the one or more of the cancer neoantigens may be an immunogenic composition, a composition comprising immune cells or a therapeutic antibody. The immunogenic composition may comprise one or more of the candidate peptides (such as e.g. a neoantigen peptide or protein or a cell displaying the neoantigen). The composition comprising immune cells may comprise T cells, B cells and/or dendritic cells. The composition comprising a therapeutic antibody may comprise one or more antibodies that recognise at least one of the one or more of the candidate peptides. An antibody may be a monoclonal antibody.

Designing an immunotherapy that targets one or more of the cancer neoantigens identified may comprise designing one or more candidate peptides for each of the one or more neoantigens targeted, each peptide comprising at least a portion of a neoantigen targeted. The method may further comprise obtaining the one or more candidate peptides. The method may further comprise testing the one or more candidate peptides for one or more further properties (in addition to the outcome of production of the one or more candidate peptides as described herein). Further testing may be performed in vitro or in silico. For example, the one or more peptides may be tested for immunogenicity, propensity to be displayed by MHC molecules (optionally by specific MHC molecule alleles, where the alleles may have been chosen depending on the MHC alleles expressed by the subject), ability to elicit proliferation of a population of immune cells, etc. The method may further comprise producing the immunotherapy. The method may further comprise obtaining a population of dendritic cells that has been pulsed with one or more of the candidate peptides. The immunotherapy may be a composition comprising T cells that recognise at least one of the one or more of the neoantigens identified. The composition may be enriched for T cells that target at least one of the one or more of the neoantigens identified. The method may comprise obtaining a population of T cells and expanding the population of T cells to increase the number or relative proportion of T cells that target at least one of the one or more of the neoantigens identified. The method may further comprise obtaining a T cell population. A T cell population may be isolated from the subject, for example from one or more tumour samples obtained from the subject, or from a peripheral blood sample or a sample from other tissues of the subject. The T cell population may comprise tumour infiltrating lymphocytes. T cells may be isolated using methods which are well known in the art. For example, T cells may be purified from single cell suspensions generated from samples on the basis of expression of CD3, CD4 or CD8. T cells may be enriched from samples by passage through a Ficoll-paque gradient. The method may further comprise expanding the T cell population. For example, T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28. The T cells may be co-cultured with antigen-presenting cells (APCs), which may have been irradiated. The APCs may be dendritic cells or B cells. The dendritic cells may have been pulsed with the candidate peptides (containing one or more of the identified neoantigens) as single stimulants or as pools of stimulating neoantigen peptides. Expansion of T cells may be performed using methods which are known in the art, including for example the use of artificial antigen presenting cells (aAPCs), which provide additional co-stimulatory signals, and autologous PBMCs which present appropriate peptides. Autologous PBMCs may be pulsed with peptides containing neoantigens as discussed herein as single stimulants, or alternatively as pools of stimulating neoantigens. Also described herein is a method for expanding a T cell population for use in the treatment of cancer in a subject, the method comprising: producing one or more neoantigen peptides using a method as described herein, such as a method according to any embodiment of the second aspect; obtaining a T cell population comprising a T cell which is capable of specifically recognising one of the neoantigen peptides; and co-culturing the T cell population with a composition comprising the neoantigen peptide. The method may have one or more of the following features. The method may further comprise identifying the one or more neoantigen peptides. The T cell population obtained may be assumed to comprise a T cell capable of specifically recognising one of the neoantigen peptides. The method preferably comprises identifying a plurality of neoantigen peptides. The neoantigen peptides may comprise one or more clonal neoantigens. The T cell population may comprise a plurality of T cells each of which is capable of specifically recognising one of the plurality of neoantigen peptides, and co-culturing the T cell population with a composition comprising the plurality of neoantigen peptides. The co-culture may result in expansion of the T cell population that specifically recognises one or more of the neoantigen peptides. The expansion may be performed by co-culture of a T cell with the one or more neoantigen peptides and an antigen presenting cell. The antigen presenting cell may be a dendritic cell. Thus, the expansion may be a selective expansion of T cells which are specific for the neoantigen peptides. The expansion may further comprise one or more non-selective expansion steps. Thus, also described herein is a composition comprising a population of T cells obtained or obtainable by a method as described above.

Thus, the disclosure also provides a T cell composition comprising a T cell population selectively enriched with T cells that recognise one or more neoantigens, preferably clonal neoantigens, wherein the T cell population has been selectively enriched using peptides that have been produced using any of the methods described herein.

In a T cell composition as described herein the expanded population of neoantigen-reactive T cells may have a higher activity than the population of T cells which have not been expanded, as measured by the response of the T cell population to restimulation with a neoantigen peptide. Activity may be measured by cytokine production, and wherein a higher activity is a 5-10 fold or greater increase in activity.

References to a plurality of neoantigens may refer to a plurality of peptides or proteins each comprising a different tumour-specific mutation that gives rise to a neoantigen. Said plurality may be from 2 to 250, from 3 to 200, from 4 to 150, or from 5 to 100 tumour-specific mutations, for example from 5 to 75 or from 10 to 50 tumour-specific mutations. Each tumour-specific mutation may be represented by one or more neoantigen peptides. In other words, a plurality of neoantigens may comprise a plurality of different peptides, some of which comprise a sequence that includes the same tumour-specific mutation (for example at different positions within the sequence of the peptide, or within peptides of varying lengths). Thus, the one or more selected peptides obtained at step 216 (or any method comprising selecting peptides using the methods described herein) may comprise from 2 to multiple hundred peptides, such as e.g. between 2 and 400 peptides, between 2 and 300 peptides, between 2 and 250 peptides, between 2 and 200 peptides, between 10 and 400 peptides, between 10 and 300 peptides, between 10 and 250 peptides, between 10 and 200 peptides, between 50 and 400 peptides, between 50 and 300 peptides, between 50 and 250 peptides, or between 50 and 200 peptides. In particular, the one or more selected peptides may comprise up to a maximum number of peptides that is set by the capacity of a synthesis process of or a step thereof, such as for example the number of wells in a reaction plate used for a single synthesis run or a multiple thereof. For example, when 96 wells plates are used the number of selected peptides may be set to a maximum of 96, 192, 288, or 384. Instead or in addition to this, in the context of peptides comprising a different tumour-specific mutation that gives rise to a neoantigen, the number of peptides selected may be set to a maximum corresponding to the number of tumour-specific mutations that give rise to a neoantigen identified in a subject, or to the number of different peptides of a predetermined length that comprise said tumour-specific mutations. For example, as many as 1,000 to 10,000 peptides comprising one or more coding mutations may be identified and peptides comprising each of said mutations may be selected using the methods described herein. A T cell population that is produced in accordance with the present disclosure will have an increased number or proportion of T cells that target one or more neoantigens that are represented in peptides selected using the methods described herein. That is to say, the composition of the T cell population will differ from that of a “native” T cell population (i.e. a population that has not undergone the expansion steps discussed herein), in that the percentage or proportion of T cells that target a neoantigen that is produced as described herein will be increased. The T cell population according to the disclosure may have at least about 0.2, 0.3, 0.4, 0.5, 0 6, 0 7, 0 8, 0 9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen for which a peptide is produced as described herein.

The immunotherapies described herein may be used in the treatment of cancer. Thus, the disclosure also provides a method of treating cancer in a subject comprising administering an immunotherapeutic composition as described herein to the subject.

Suitably, in any embodiment of any aspect described herein, the cancer may be ovarian cancer, breast cancer, endometrial cancer, kidney cancer (renal cell), lung cancer (small cell, non-small cell and mesothelioma), bladder cancer, gastric cancer, oesophagal cancer, colorectal cancer, cervical cancer, endometrial cancer, brain cancer (gliomas, astrocytomas, glioblastomas), melanoma, merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas. For example, the cancer may be lung cancer, such as lung adenocarcinoma or lung squamous-cell carcinoma. As another example, the cancer may be melanoma. The cancer may be bladder cancer. The cancer may be head and neck cancer. In embodiments, the cancer may be selected from melanoma, merkel cell carcinoma, renal cancer, non-small cell lung cancer (NSCLC), urothelial carcinoma of the bladder (BLAC) and head and neck squamous cell carcinoma (HNSC) and microsatellite instability (MSI)-high cancers. In some embodiments, the cancer is non-small cell lung cancer (NSCLC). In any embodiment of any aspect, the subject may be human.

Treatment using the compositions and methods of the present disclosure may also encompass targeting circulating tumour cells and/or metastases derived from the tumour. Treatment according to the present disclosure targeting one or more neoantigens, preferably clonal neoantigens, may help prevent the evolution of therapy resistant tumour cells which may occur with standard approaches such as chemotherapy, radiotherapy, or non-specific immunotherapy. The methods and uses for treating cancer described herein may be performed in combination with additional cancer therapies. In particular, the T cell compositions described herein may be administered in combination with immune checkpoint intervention, co-stimulatory antibodies, chemotherapy and/or radiotherapy, targeted therapy or monoclonal antibody therapy. ‘In combination’ may refer to administration of the additional therapy before, at the same time as or after administration of the T cell composition as described herein.

The invention also provides a method for producing an immunotherapeutic composition, the method comprising predicting the outcome of production of one or more candidate peptides each comprising a neoantigen, selecting one or more peptides from the candidate peptides based on the predicting, and producing an immunotherapeutic composition that targets the neoantigen(s).

Also described herein is a composition comprising a neoantigen peptide, neoantigen peptide specific immune cell, or an antibody that recognises a neoantigen peptide, for use in the treatment or prevention of cancer in a subject, wherein said neoantigen peptide has been identified using the methods described herein. Also described herein is a composition comprising a neoantigen peptide, neoantigen peptide specific immune cell, or an antibody that recognises a neoantigen peptide, wherein said neoantigen peptide has been produced using the methods described herein. Also described herein is a neoantigen peptide, immune cell which recognises a neoantigen peptide, or antibody which recognises a neoantigen peptide, for use in the treatment or prevention of cancer in a subject, wherein said neoantigen peptide has been produced using the methods described herein. Also described herein is the use of a neoantigen peptide, immune cell which recognises a neoantigen peptide, or antibody which recognises a neoantigen peptide, in the manufacture of a medicament for use in the treatment or prevention of cancer in a subject, wherein said neoantigen peptide has been produced using the methods described herein. Also described herein is a method of treating a subject that has been diagnosed as having cancer, the method comprising administering an immunotherapy that has been provided using the methods described herein, or a composition as described herein.

### Systems

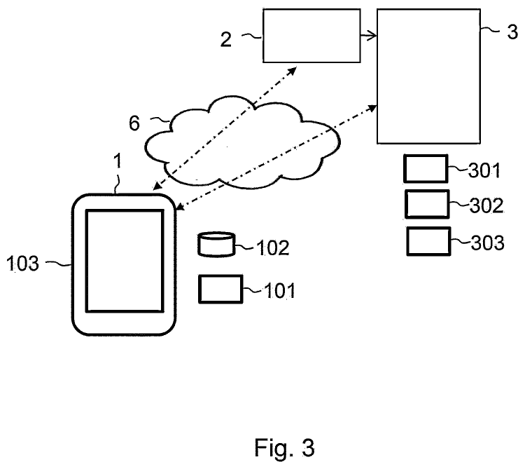

FIG. 3 shows an embodiment of a system for predicting the outcome of manufacture of peptides by chemical synthesis, and/or providing an immunotherapy based at least in part on the peptides to be manufactured, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network 6, to peptide synthesis means 3, such as a peptide synthesizer, and/or to one or more databases 2 storing peptide data. The one or more databases may additionally store other types of information that may be used by the computing device 1, such as e.g. manufacturing constraints, product constraints, parameters, etc. The computing device may be a smartphone, tablet, personal computer or other computing device. The computing device is configured to implement a method for predicting the outcome of peptide manufacturing, as described herein. In alternative embodiments, the computing device 1 is configured to communicate with a remote computing device (not shown), which is itself configured to implement a method of predicting the outcome of peptide manufacturing, as described herein. In such cases, the remote computing device may also be configured to send the result of the method to the computing device. Communication between the computing device 1 and the remote computing device may be through a wired or wireless connection, and may occur over a local or public network such as e.g. over the public internet or over WiFi.

The peptide synthesis means 3 may be in wired connection with the computing device 1, or may be able to communicate through a wireless connection, such as e.g. through a network 6, as illustrated. The peptide synthesis means 3 may be located in a physically separate location. The connection between the computing device 1 and the peptide production means 3 may be direct or indirect (such as e.g. through a remote computer). The peptide production means 3 are configured to synthesize peptides by chemical synthesis, for example solid phase peptide synthesis using a batch method (as opposed to a flow method). The peptide production means 3 may be connected to or comprise one or more means for characterising the output of a manufacturing process 301, such as a high pressure liquid chromatography (HPLC) and/or a mass spectrometry machine. In some embodiments, the output of the manufacturing process (typically, a solution comprising the peptides that have been manufactured) may manually or automatically be assessed by HPLC and/or mass spectrometry. Preferably, the output of the manufacturing process is assessed by HPLC. Any method that is suitable for use in the determination of the composition of a peptide sample may be used within the context of the present invention. The means for characterizing the output of the manufacturing process 301 may be provided as a physically separate instrument, which may be located in a different location from the peptide synthesis means 3 and/or the computing device 1. The means for characterizing the output of the manufacturing process may be operatively connected to the peptide synthesis means 3 and/or the computing device 1 and/or the one or more databases 2. The peptide production means 3 may further be connected to or comprise one or more sensors 302 for measuring one or more parameters of the production process. The peptide production means 3 may be in direct or indirect connection with one or more databases 2, on which the outcome of the manufacturing process and/or one or more parameters of the manufacturing process (raw or partially processed) may be stored. The peptide production means 3 may further be connected to or comprise one or more purification means 303 for purifying one or more products of the process. Any peptide purification method used in the art may be used and implemented by the peptide purification means 303. For example, the peptide purification means may comprise a prep LC (preparative liquid chromatography, also referred to as preparative HPLC) system, a desalting system (whereby a sample is for example loaded on a silica plate and washed to get rid of non-peptide impurities), a catch-and-release peptide purification system such as that available from Belyntic GmbH (a system based on PEC linkers that selectively catch a product of desired length, performed in a 96 well plate format), for example a system as described in EP3408257A1, or others.

The following is presented by way of example and is not to be construed as a limitation to the scope of the claims.

### EXAMPLES

These examples describe the training and use of machine learning models for predicting the outcome of manufacture of peptides by chemical synthesis.

### Data

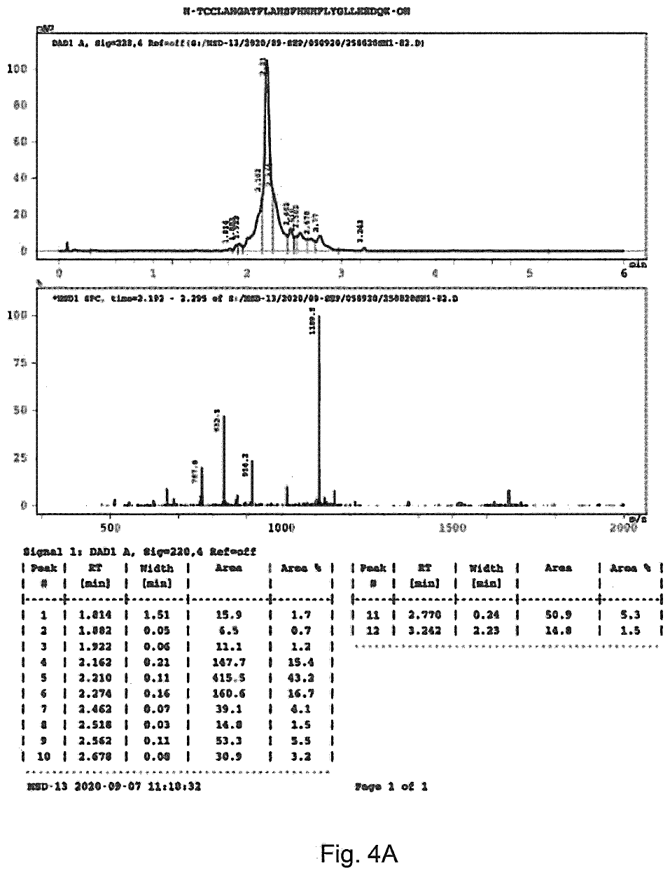

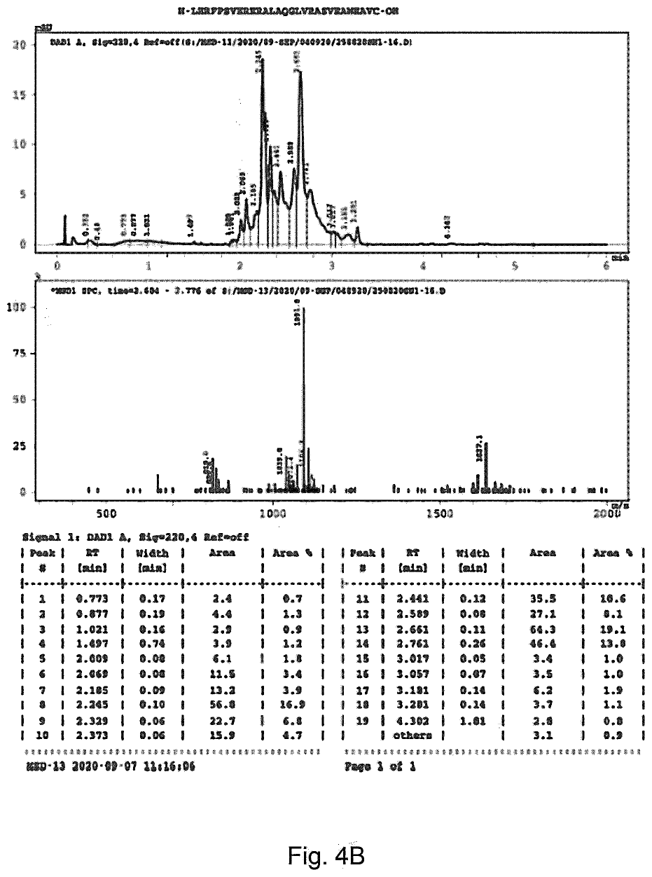

The data used in these examples comprises metrics derived from chromatography data (from HPLC—in particular, reverse-phase C-18 columns were used, with 100 A pore size, 2-5 μm particle size) related to the product of chemical synthesis of a total of 8,810 peptides of 29 amino acids, manufactured by two different manufacturers labelled as “MFG1” and “MFG2”. The characteristics of this data are shown in Table 1. In Table 1, “purity” refers to the relative area of the chromatographic peak corresponding to the full-length peptide and the area of all other chromatographic peaks quantified. In table 1, the “pass rate” refers to the proportion of peptides in the batch that satisfy a pass/fail criterion. In this example, the pass-fail criterion was selected as whether the % HPLC area of the chromatogram peak with the desired molecular weight (full length peptide, in this case) is >5% of the total % area of the chromatogram (i.e. purity as described above >5%). Other pass/fail criteria that can be used may include whether the peak corresponding to the desired peptide is the largest peak in the chromatogram, whether the peak corresponding to the desired peptide represents more than 10% of the % area in the chromatogram, amongst others. These concepts are illustrated in the example chromatograms on FIG. 4, where FIG. 4A shows a chromatogram for a high purity peptide and FIG. 4B shows a chromatogram for a low purity peptide (both passing the pass/fail criterion). On FIG. 4A, it can be seen that 12 chromatographic peaks were quantified, but there is a clear dominant peak (peak number 5) with a mass to charge ratio of 1109.5 (where in this case the peak corresponds to a triple charged peptide, i.e. ([M+H]3+), of a peptide with molecular weight of 3326.7 g/mol, the signal at 832.5 corresponding to the quadruple charged ion ([M+H]4+) for the same peptide) having the largest % area (43.2%). On FIG. 4B, it can be seen that 19 peaks were quantified, with several peaks having large % area (in particular peaks 8, 13 and 14, respectively with 16.9%, 19.1% and 13.8%), where the peak having the molecular weight of interest corresponds to peak 13 (i.e. not the dominant peak). These shorter peaks correspond to incomplete reaction products, i.e. shorter versions of the desired sequence. The peptide on FIG. 4A has a purity of 43.2% whereas the peptide in FIG. 4B has a purity of 19.1%. Both satisfy the pass/fail criterion described above.

TABLE 1
Summary statistics for purity and pass rate data.

| | MFG1 | MFG2 |
|---|---|---|
| Batch count | 48 | 21 |
| Number of observations | 6733 | 2077 |
| Average purity | 41% | 45% |
| Pass rate | 79% | 85% |

No chromatographic data was available for the peptides that do not satisfy the pass/fail criterion described above, and a purity of 0 was therefore assigned to all of these peptides. Each batch refers to a set of peptides designed for a single patient. In the particular data used here, batches have an average size of approximately 50 to 150 peptides. Batches are run by the manufacturer in 96 wells plates.

For some peptides in the data from MFG1, chromatography was not possible because the peptide synthesised was too insoluble. In such cases, mass spectrometry was used to detect the peptides and the purity was inferred from the size of the mass spectrometric peak. This is reported with a lower level of accuracy than the chromatographic peak area, in this case rounder to the nearest 10%.

The data was cleaned to remove duplicates and put it in a common standard format. For each manufacturer data set, 4 or 5 batches were held back for testing of the models. The test set was defined by batch (rather than by sampling across batches) because strong batch to batch variability was observed (see below).

### Methods

All code was written in Python 3.9.5. Scikit-Learn 0.24.2 (Pedregosa et al., 2011) was used for the majority of non-neural modelling, cross validation and score calculating. Pandas 1.2.4 (McKinney, 2010) and NumPy 1.20.2 (Harris, 2020) were used to load and clean the data. Statsmodels 0.12.2 (Seabold & Perktold, 2010) was used for linear mixed effects models. Neural models were all written in PyTorch 1.9.0 (Paszke et al., 2019). All experiments were carried out on a single AWS G3 instance comprising an 8 core Intel Xeon CPU and a 8 gig NVIDIA Tesla M60 GPU. The BioPython (Cock et al., 2009) and Pyteomics (Goloborodko et al., 2013) packages were used to obtain some peptide features.

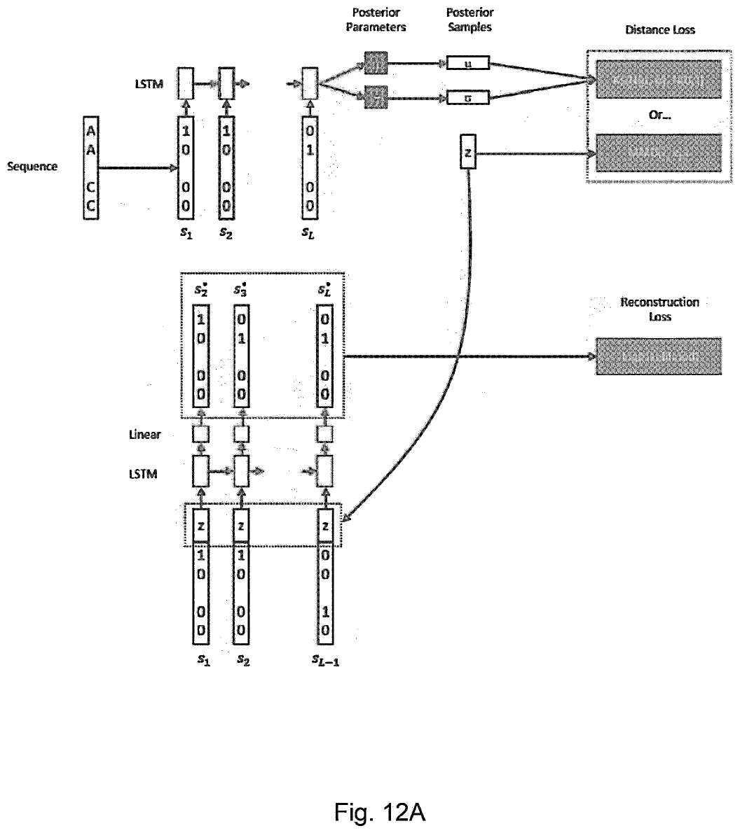

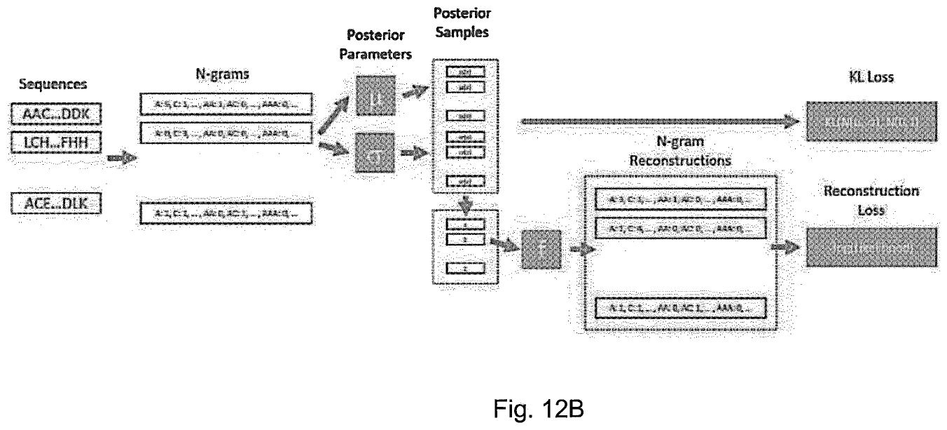

Variational autoencoders (VAEs): Variational autoencoders are trained by minimising the loss function provided by:

$$\frac{N}{M} {\sum }_{i = 1}^{M} ( log {p}_{θ} ( {x}_{i} \vert {z}_{i} ) - λ * {D}_{KL} ( log q ( {z}_{i} \vert {x}_{i} ) ‖ p ( {z}_{i} ) )$$

where i is the training minibatch of size M, N is the dataset size, zᵢ are samples from the latent space (dimension K), using a K dimensional unit Gaussian ε:
zᵢ=g_{Φ}(εᵢ, x)=μ(xᵢ)+Σ(xᵢ)*εᵢ where μ and Σ are encoding networks of variable architecture, log p_{θ}(xᵢ|zᵢ) is a probability distribution that depends on the application and parameterised by some decoding function of f_{θ}(zᵢ), again of variable architecture, D_{KL} is the Kubek Leibler (KL) divergence between two probability distributions, that effectively regularises the autoencoder. λ controls the strength of the KL regularisation. The sequential VAE used in the examples below is a VLSTM (very long short term memory network) based on a model in Das et al. (2020). It uses sequential encoder and decoders. μ and Σ are LSTM networks that operate on one-hot representations of the peptides. The decoding network is also conditional not only on the latent state zᵢ, but also on earlier steps in the sequence, indexed by n, which influence the distribution log p_{θ} via decoding LSTM parameters: log p_{θ}(xᵢ|zᵢ)=log p(x_{i,n}|f_{θ}(x_{i,n-1}, x_{i,n-2} . . . , zᵢ)). This network decodes as a LSTM using the previous state of the sequence, concatenated with the hidden state, at every step. The conditional probability distribution used above for x is then a product of L categorical distributions with dimension equal to V, where L is the sequence length and V is the vocab size. The network parameterises the probability of each element of the vocab at each step. The schematic architecture of this model (and the WLSTM model discussed below) is illustrated on FIG. 12A. The encoder (top branch) learns a latent state z that is used at each step by the decoder (bottom branch). The encoder branch takes as input a sequence and produces as output a latent vector h which is projected into vectors p and a using a fully connected layer. These are used together with a vector of independent identically distributed variables to construct a random vector z. The latent vector z is a random vector conditioned on the input, rather than a deterministic output for a given input sequence. The decoder samples output sequences conditional on a given latent vector z. At each step of the decoder, the previous element in the sequence and the latent vector z are used as a concatenated input. More details on the architecture can be found in Ha and Eck, 2017. V or W LSTMs are configured by changing the distance loss (top right): the VAE is similar to a standard autoencoder except that it includes a regularisation term minimising the KL loss; a WAE similarly includes a regularisation term but that term minimises the sampled MMD distance. Thus, depending on the loss term use (KL or MMD), the autoencoder depicted on FIG. 12A functions as a VAE or a WAE. Both of these models are sequential probabilistic models, where the latent state gets fed into the decoder at every step. The decoder works much like a language model, however it is forced to use the latent state to encode information about the sequences.

Another VAE was looked at that was based on the Neural Variational Document Model (NVDM) in Miao et al. 2016. Sequences are represented as n-gram vectors of size V. Here, the encoder networks μ and Σ are multi-layer neural networks, as is the decoder f_{θ}. The conditional probability distribution is a multinomial of dimension V and the decoder network parameterises the probability of each n-gram occurring. The architecture of this model is illustrated on FIG. 12B. It is similar to the VAE studied in Kingma et al. (2013), except the reconstruction probability is a multinomial distribution. This is similar to a traditional autoencoder where an image is reconstructed using the latent state only. Thus, this model does not use the latent state at every step.

Sequential WAEs: A sequential WAE, or WLSTM, was also studied. In a WAE, the problem of training an autoencoder is re-cast as an optimal transport problem, minimizing the Wasserstein distance between the true and decoded probability distributions. This problem can be re-written as minimizing the expected distance between true and decoded data subject to the marginal on the posterior (over the data) on z being equal to its prior. This constraint is then relaxed into a penalty on some distance measure between the marginal posterior and the prior, leading to the WAE objective:

$$\frac{N}{M} {\sum }_{i = 1}^{M} ( log {p}_{θ} ( {x}_{i} \vert {z}_{i} ) - λ * N * {D}_{MMD} )$$

A maximum discrepancy distance measure (MMD) was used as in Tolstikhin et al. (2017):

$${D}_{MMD} = \frac{1}{M ( M - 1 )} {\sum }_{l \neq j}^{M} k ( {z}_{l} , {z}_{j} ) + \frac{1}{M ( M - 1 )} {\sum }_{l \neq j}^{M} k ( {\hat{z}}_{l} , {\hat{z}}_{j} ) - \frac{2}{{M}^{2}} {\sum }_{l , j}^{M} k ( {\hat{z}}_{l} , {\hat{z}}_{j} )$$

which is an empirical estimate for the distance between two distributions, measuring the difference between two distributions in a Reproducing Kernel Hilbert Space defined by a user supplied kernel k(.,.) —in this case the inverse multiquadratics kernel

$$k ( x , y ) = \frac{C}{C + {‖ x - y ‖}_{2}^{2}} ,$$

where \hat{z}_{l,j} are samples from the approximated posterior and z are samples from the prior, C=2σ₀² where σ₀ is a hyperparameter controlling the width of the kernel. The main difference between VAEs and WAEs comes from the regularisation term in the loss. For the VAE, all elements of the minibatch posterior are regularised back to the prior. For the WAE, the batch as a whole is regularised. So, observations can trade off distance to the prior. The WAE is less restrictive on the posterior. In practice, this means the VAE encodes with more latent variance than the WAE.

Training of unsupervised models: The VAE and WAE deep learning models described above are used below to learn additional features by unsupervised learning. For this purpose the models were trained on human proteome data (Pruitt et al., 2007). Using proteome data is a departure from Das et al. (2020), where naturally occurring peptides are used. The sampled peptidome was thought to be a better training set for this problem as it more closely matches the peptides manufactured in the data available, which represent peptides containing mutations identified in individual cancer patients. Assuming that mutations occur randomly in the genome, random sampling from the proteome rather than using a biased subset of the proteome captured by expressed peptide may more accurately reflect the characteristics of the peptides at hand. However, both approaches are likely to be useful, and choice of an approach may depend on context. A random sample of 5000 peptides was used to train these models. Other sample sizes were tested with little effect.

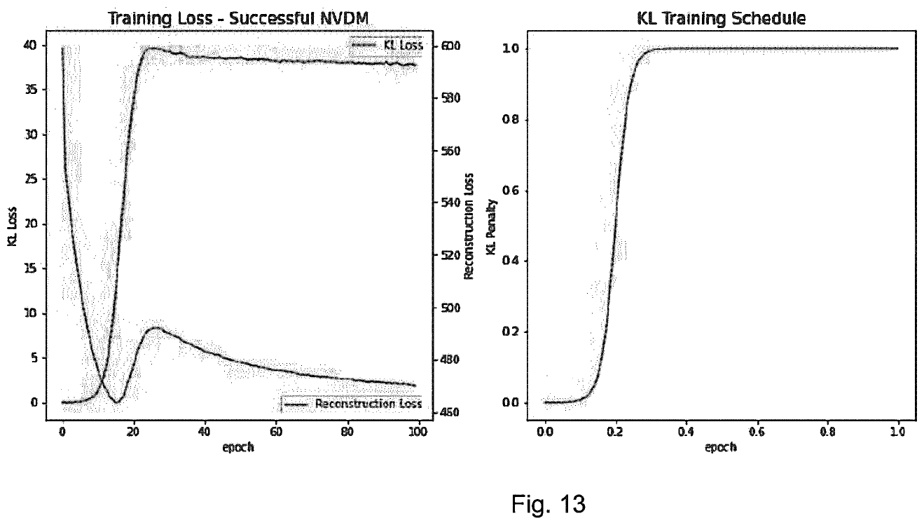

KL annealing, as set out in Bowman et al. (2015) was used to reduce the risk of settling into a local-minimum corresponding to the zero-information autoencoder, for the NVDM and the VLSTM. Using this approach, the KL penalty is increased from zero on a sigmoid schedule to some maximum, over the course of training. The network is therefore trained as a deterministic autoencoder at first, then the KL loss is slowly introduced to bring it into a regularized state, avoiding the failure mode at the local minimum. Successful application of this technique is shown in FIG. 13, where an NVDM (with 256-dimensional encoder and decoder and 128-dimensional latent state, operating on trigram data) is trained using an annealing schedule that goes to a maximum by epoch 30. Initially, the KL loss is low and the reconstruction loss descends quickly as the encoder trains deterministically. As the KL loss is turned on, the encoder is driven back towards the prior and the reconstruction loss jumps. Then, the reconstruction and KL loss slowly decay as the encoder continues to train.

Transformer models: In addition to the models above, a language model of proteins (Rives et al., 2021), based on a bidirectional transformer (Devlin et al., 2018), was used to generate features from an intermediate encoder stack. The smallest transformer was used due to hardware constraints i.e. not being able to fit larger models on the GPU. The third stack was used as it gave best cross validated performance. Although there was a performance improvement for the classification problem, it was not significantly better than that obtained from the autoencoders. It is likely that the majority of the features learned by this model, which pertain to longer chains of amino acids, are not relevant for the shorter chains studied.

### Results

### Batch to Batch Variability

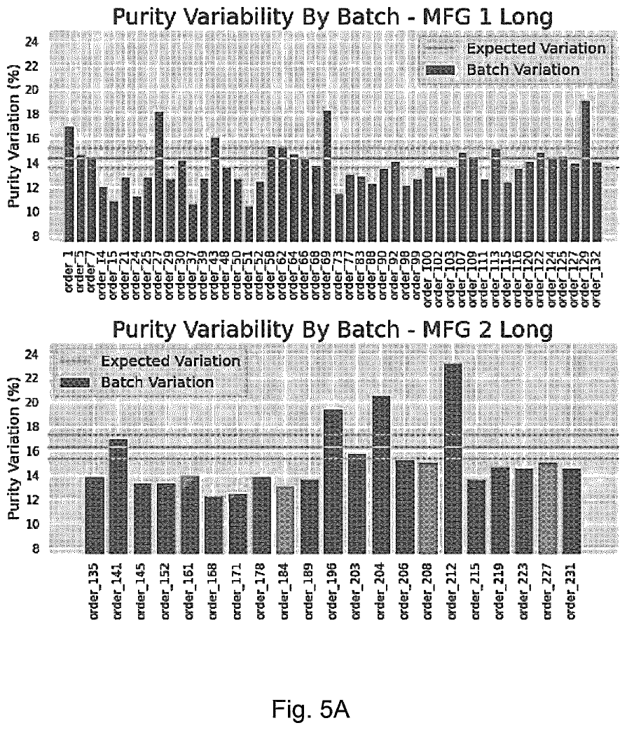

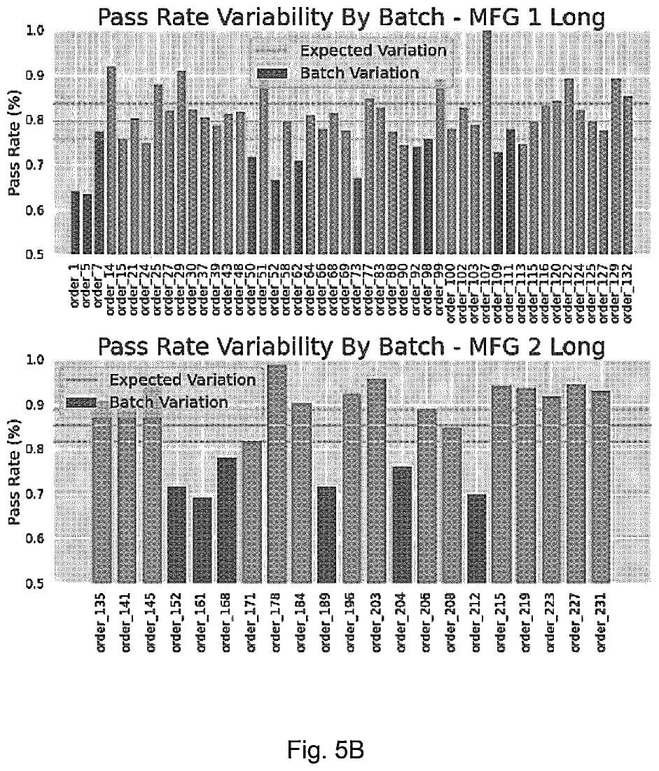

Batch to batch variability in terms of purity was investigated by calculating the within batch purity variation (standard deviation of non-zero purity values within a batch). This was compared to a standard deviation of purity over all observations (across batches—referred to as “expected variation”) and its variability estimated by repeatedly (50 times) sampling 500 purities at random from the whole data set and calculating the standard deviation from these. The results of this are shown on FIG. 5A (Top for MFG1, bottom for MFG2), where the expected variation is shown as a continuous line and its variability as a pair of dashed lines. Batch to batch variability in terms of pass/fail % was investigated by calculating the average pass rate across batches (expected pass rate) and the variability around that by repeating (50 times) sampling 500 peptides at random from the whole data set and calculating the standard deviation of the pass rate from these. The results of this are shown on FIG. 5B (Top for MFG1, bottom for MFG2), where the expected variation is shown as a continuous line and its variability as a pair of dashed lines. It can be seen from these plots that some batches show more variation in purity and pass rate than others, and more than would be expected if the batches all came from identical processes.

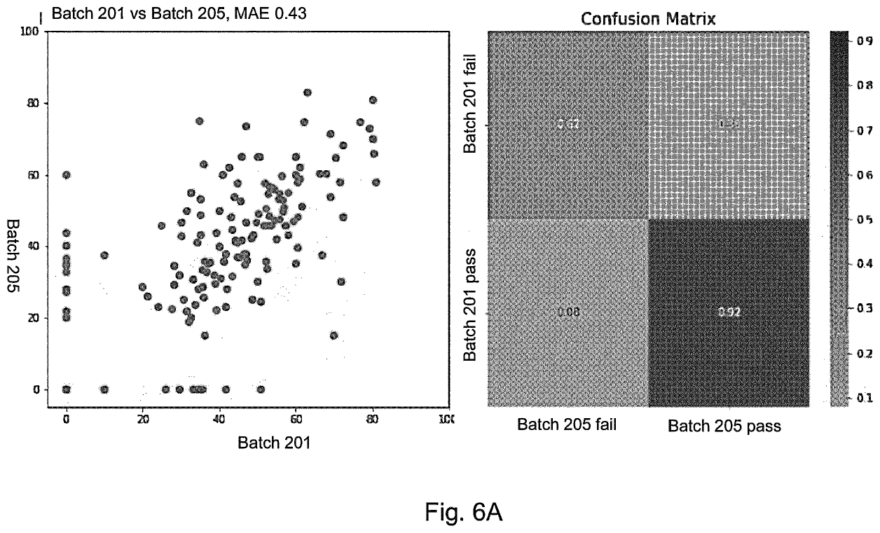

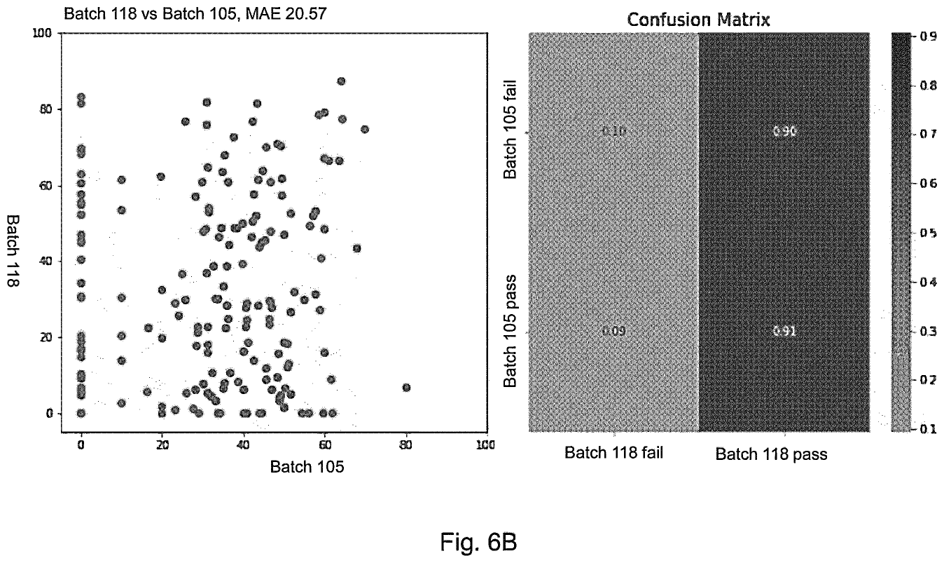

Batch to batch variability was also investigated by comparing 2 pairs of repeat batches, one pair repeated by the same manufacturer and one pair repeated by a different manufacturer. The results of this are shown on FIG. 6A (same manufacturer) and 6B (different manufacturer), which each show, for each pair, a scatterplot of purities for each of the peptides successfully manufactured in both repeat batches in a pair (left), and a confusion matrix showing the number of peptides that passed or failed in the respective batches. For the data on FIG. 6A, the mean absolute error of predicting purity in one batch from purity in the other is 10.4%. The peptides that passed in one batch generally passed in the other batch (92% of the pass in first batch also pass in the second one), but there is less consistency in the failed peptides (62% of the peptides that failed in the first batch also failed in the second batch). For the data on FIG. 6B, the mean absolute error was higher (20.57%) and the confusion matrix also shows lower consistency. The data indicates that there is some variability in peptide purity and which peptides fail, which may not be explainable even using a perfect sequence-based model. However, there is also some predictability, particularly within data from the same manufacturer, which an appropriate model may be able to capture (and was in fact able to capture as demonstrated below). The data further indicates that models are likely to perform better at predicting the outcome of manufacture for processes that are similar to those with which the training data was obtained. For all subsequent work, models were trained and tested using data from the same manufacturer.

The data was also examined for possible systematic effects (above expected variability) associated with the position of the peptides on the slides used for manufacture, but no such effect was found.

### Linear Models

The data described above was first used to train linear regression (for prediction of purity) and classification (for prediction of pass/fail) models. They are easy to implement, robust, errors caused by data issues can be spotted quickly and the models can be interpreted straightforwardly. They require features to be manually engineered; features based on the global characteristics of the sequences were used, as further described below. These models showed that the features that are most important to the model depended on the manufacturer, and hence are likely to be process-dependent at least to some extent. This is in agreement with the indications from the batch variability study. For all manufacturers and both the prediction of purity and pass/fail, models were identified that could satisfactorily predict the outcome of peptide manufacture. This was quantified using the mean square error (MSE) for regressions and the area under the curve (AUC) for classifications.

A number of features of potential relevance based on expert knowledge were included in the models (also referred to herein as “expert defined features”). These included:
the aggregation parameters P_{agg} (Krchnak et al., 1993—provided in Table 2, this quantifies the propensity of residues to be present in chains that aggregate as measure by a swellometer, this is calculated as an average over the chain), although the use of P*_{c} as described in Milton et al., 1990 was also investigated (a parameter calculated as the average over the chain of P_{c}=1/(0.739 Pₐ+0.345 P_{b}) where Pₐ, P_{b} are the Chou-Fassman parameters for each amino acid in the chain, where the Chou-Fassman parameters Pₐ, P_{b} and Pₜᵤᵣₙ (see Table 2) empirically quantify the preference for amino acids being in alpha helix, beta sheets or turns),
the average solubility score of a sequence (calculated as described in Narita et al., 1984, as an average of the solubility scores S in Table 2),
the % content of each of the 20 canonical amino acids (listed in Table 2) in the chain (calculated using the Bio.SeqUtils.ProtParam module in Biopython),
the secondary structure fraction (fraction of amino acids which tend to be in helix, turn or sheet as calculated in the Bio.SeqUtils.ProtParam module in Biopython with amino acids in helix: V, I, Y, F, W, L.; amino acids in Turn: N, P, G, S.; amino acids in sheet: E, M, A, L),
the predicted density, charge at pH=7, isoelectric point and chromatographic retention time of the peptides (all of which could affect the solubility and the behaviour of the peptide in the chromatography, which is used to quantify the output of the manufacturing process—calculated using Pyteomics for the retention time and density, and the Bio.SeqUtils.ProtParam module in Biopython for the charge and isoelectric point),
the predicted molar extinction coefficient (mec), hydrophobicity (this was quantified using both the aromaticity as defined in Lobry and Gautier (1994) which quantifies the relative frequency of Phe+Trp+Tyr, as well as the GRand AVerage of hydropathicitY (gravy) according to Kyte and Doolittle, 1982—this represents the average hydropathy values for all amino acids in the sequence, where the values H for individual amino acids are provided in Table 2), instability (Guruprasad et al., 1990) and flexibility (Vihinen et al, 1994) of the peptides (all of which were also hypothesised to affect at least solubility and also potentially the behaviour of the peptide during synthesis—all calculated using the Bio.SeqUtils.ProtParam module in Biopython).

All features were standardised, i.e. z-scored, prior to use. Standardisation was done one the training set and the test set separately.

TABLE 2
Chou-Fassman, Krchnak et al., Narita et al.
and Kyte & Doolittle parameters

| Amino acid | P(a) | P(b) | P(turn) | Pagg | S | H |
|---|---|---|---|---|---|---|
| Alanine | 1.42 | 0.83 | 0.66 | 1.32 | 0.70 | 1.8 |
| Arginine | 0.98 | 0.93 | 0.95 | 0.49 | 1.04 | −4.5 |
| Aspartic Acid | 1.01 | 0.54 | 1.46 | 0.73 | 1.20 | −3.5 |
| Asparagine | 0.67 | 0.89 | 1.56 | 0.96 | 1.35 | −3.5 |
| Cysteine | 0.70 | 1.19 | 1.19 | 1.07 | 1.18 | 2.5 |
| Glutamic Acid | 1.51 | 0.37 | 0.74 | 1.16 | 0.83 | −3.5 |
| Glutamine | 1.11 | 1.10 | 0.98 | 0.82 | 0.86 | −3.5 |
| Glycine | 0.57 | 0.75 | 1.56 | 0.96 | 1.50 | −0.4 |
| Histidine | 1.00 | 0.87 | 0.95 | 0.40 | 1.06 | −3.2 |
| Isoleucine | 1.08 | 1.60 | 0.47 | 1.53 | 0.66 | 4.5 |
| Leucine | 1.21 | 1.30 | 0.59 | 1.18 | 0.68 | 3.8 |
| Lysine | 1.14 | 0.74 | 1.01 | 1.19 | 0.98 | −3.9 |
| Methionine | 1.45 | 1.05 | 0.60 | 1.34 | 0.58 | 1.9 |
| Phenylalanine | 1.13 | 1.38 | 0.60 | 1.16 | 0.71 | 2.8 |
| Proline | 0.57 | 0.55 | 1.52 | 0.49 | 1.59 | −1.6 |
| Serine | 0.77 | 0.75 | 1.43 | 0.89 | 1.32 | −0.8 |
| Threonine | 0.83 | 1.19 | 0.96 | 1.10 | 1.07 | −0.7 |
| Tryptophan | 1.08 | 1.37 | 0.96 | 0.91 | 0.75 | −0.9 |
| Tyrosine | 0.69 | 1.47 | 1.14 | 1.03 | 1.06 | −1.3 |
| Valine | 1.06 | 1.70 | 0.50 | 1.45 | 0.62 | 4.2 |

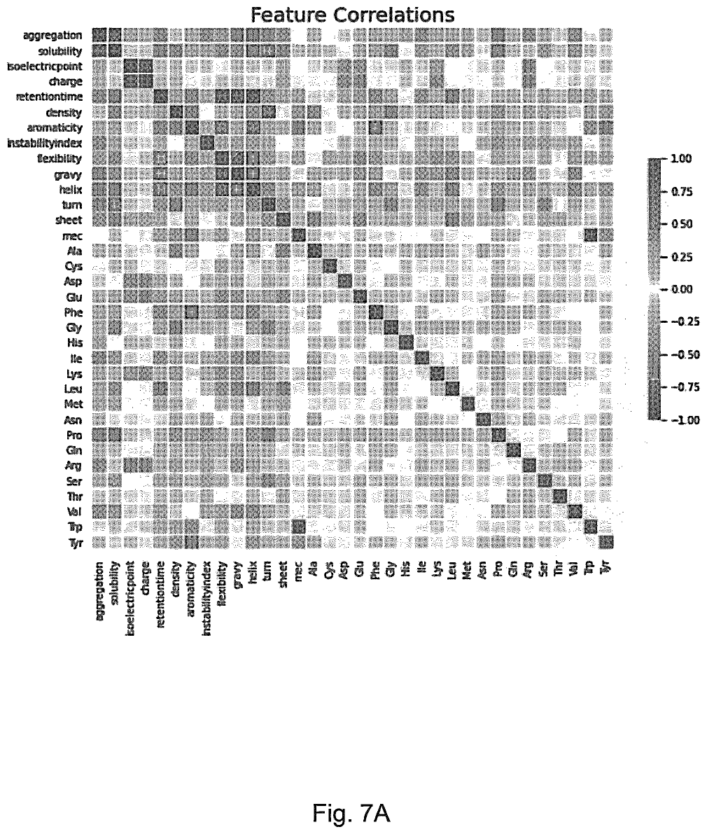

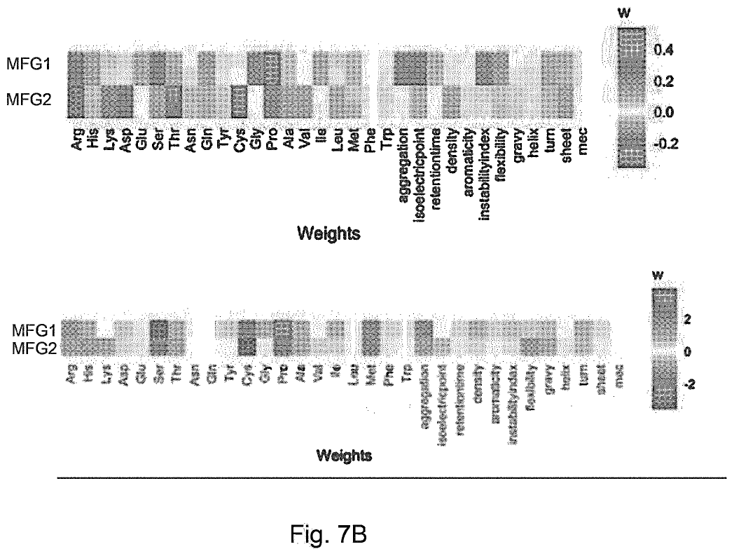

FIG. 7A shows the correlations between features used, for the data from MFG1 (a very similar plot was obtained for MFG2). This shows that there is some redundancy in the features investigated, since some are strongly correlated. For example, there is a strong negative correlation (approx. −0.8) between aggregation and solubility, and a strong positive correlation between isoelectric point and charge (approx. +0.8). This underlines the fact that not all features may be necessary or useful to provide a prediction, and different models may be built using feature selection processes (e.g. exclusion of highly correlated features, regularisation as shown below, or other feature selection processes known in the art) to select the most predictive features for a particular situation.

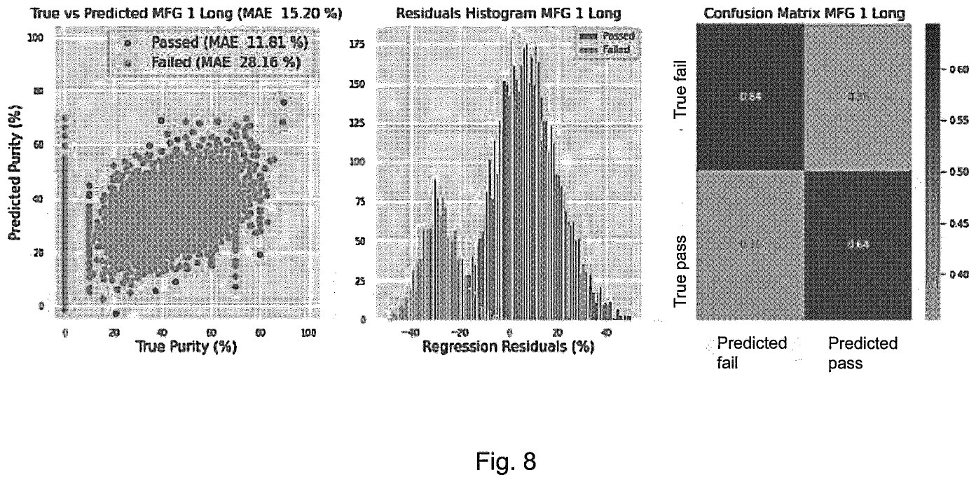

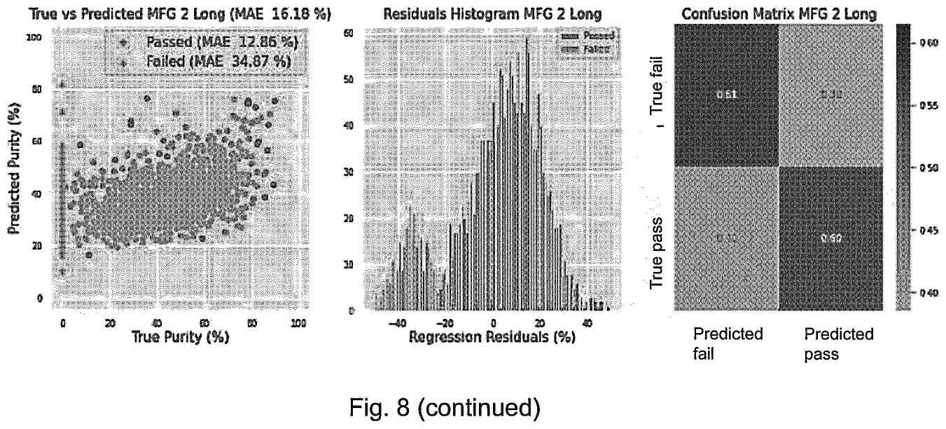

As a first step, ridge regression and L2 regularised logistic regression models were fitted to the data for each of the two manufacturers separately. Looking at the residuals of these regression models indicated that these had bimodal distributions (as shown on FIG. 8), with a mode reflecting the presence of the peptides for which a zero purity was imputed. As can be seen on FIG. 8, the MAE was lower for those peptides with imputed purity than peptides with measured purity, indicating that the model may in fact be able to predict a purity even for peptides with low purity (or at least that the assumption that these peptides have essentially zero purity does not appear to be verified). As these correspond to peptides that have not met the pass/fail criterion, which is an output predicted by the classifier models, removal of these peptides from the regression training data set is appropriate and resulted in regression models with improved MAE (e.g. from the 15.20% illustrated on FIG. 8 for the MFG1 model to 11.81%). Thus, it was possible to predict peptide purity with a MAE of 11.81% for those peptides for which real (measured) data is available as a control of the performance of the model. This is remarkably close to the MAE of 10.4% that was observed for prediction of purity in one batch from purity in a repeat of the identical batch, indicating that the model was able to capture much of the variability that is associated with the peptides sequences rather than experimental (i.e. batch) effects.

For the classifier (L2 regularised logistic regression) models, a balanced weighting was used to calculate the penalty for the pass and fail classes, with the aim of achieving nearly equal sensitivity and specificity. In particular, the weighing on each class i was calculated as: wᵢ=n/2nᵢ where n is the total number of samples in the training set and nᵢ is the number of examples of class i. In the present data this provided a failure class of weight=4 and a pass class of weight=1.

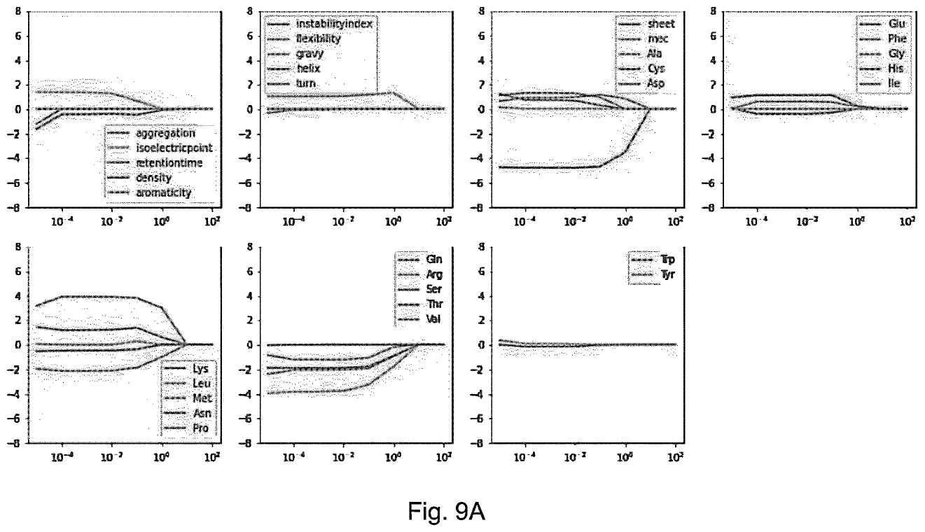

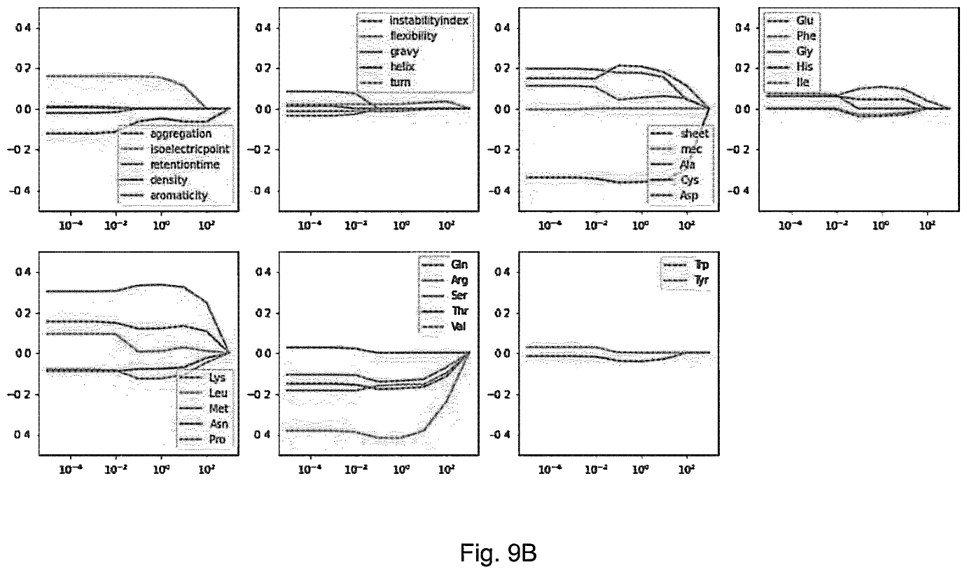

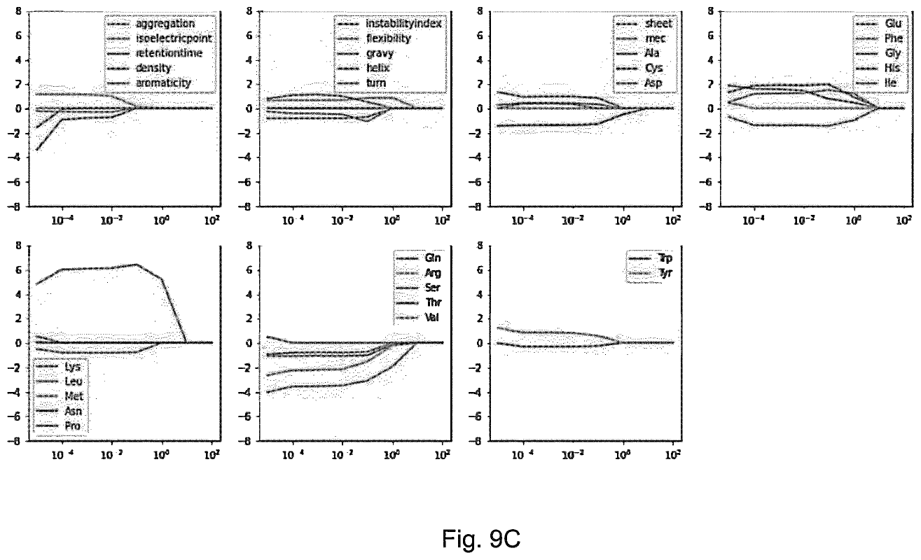

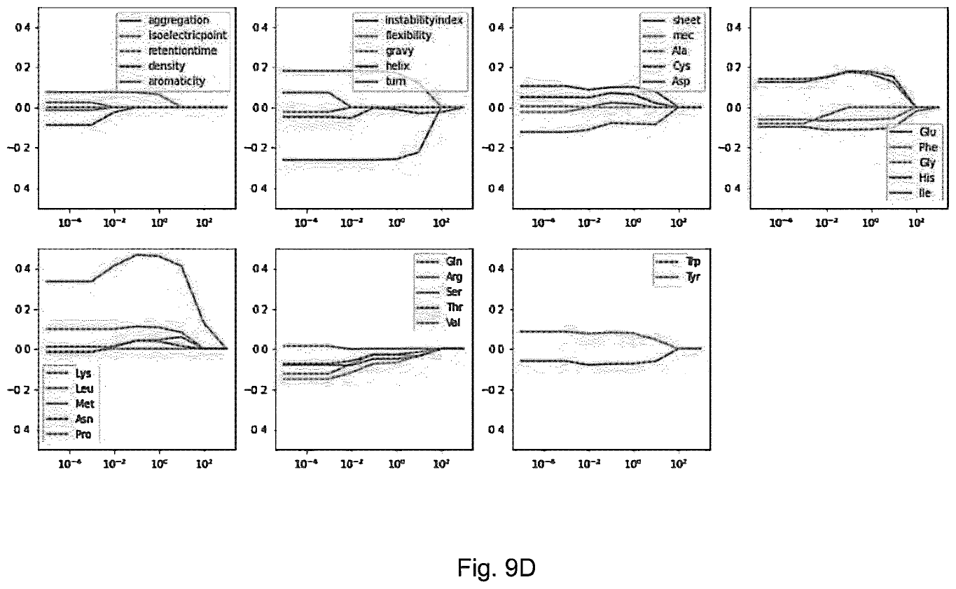

The coefficients of the trained models are provided in Table 3 and illustrated on FIG. 7B. FIG. 9 shows the change in feature importance (change in feature coefficients in the trained models) for selected features as L1 regularisation is increased in the regression and classification models, respectively for MFG1 (A, B) and MFG2 (C, D). Features that remain non-zero at higher regularisation strengths and are larger are believed to be more important. Although there is some variation, both MFG1 models agree on the importance of the % Cysteine, Proline and Arginine. Of these, only the % Proline also appears to be particularly important in the MFG2 models. The models for MFG1 have a negative coefficient for % cysteine, which is not the case for the MFG2 models, indicating that MFG2 may have been able to deal with problems associated with the presence of cysteine. All models agreed on a negative effect associated with the presence of isoleucine, serine and threonine, and a positive effect associated with the presence of alanine. Not all models had negative coefficients for alanine, valine and glutamine, all of which might have been expected to negatively affect manufacturability.

The data in Table 3 indicates that the purity prediction model for MFG1 had the following order of importance of features: Cys, Pro, Met, Ser, Lys, Arg, Flexibility, His, Ala, Thr, Aggregation, turn, Gravy, density, lie, Tyr, Gly, helix, Gln, Glu, Aromaticity, Asp, retention time, Asn, Phe, sheet, Leu, Val, mec, Trp, Instability index, Isoelectric point. The purity prediction model for MFG2 had the following order of importance of features: Pro, Ser, Cys, Aggregation, Arg, Met, Gly, lie, turn, His, Thr, Gln, density, Asp, Gravy, Glu, Ala, sheet, Flexibility, Instability index, Tyr, Aromaticity, Isoelectric point, Lys, Leu, Val, Phe, mec, retention time, Asn, Trp, helix. Thus, both models agree that the percentage of Cys, Pro, Ser, Arg and Met is highly relevant, as is the aggregation parameter though to a lower extent. In particular, the data demonstrates that a combination of simple sequence-based features such as amino acid composition metrics are likely to be more informative than single metrics such as the aggregation parameters which have been developed for the purpose of identifying “difficult” peptides.

This data indicates that the sequence-based features that are most relevant to prediction of the output of peptide synthesis likely vary depending on the particular process used (as evidenced by the different coefficients obtained for the two different manufacturer models). This indicates that the best model to predict the outcome of peptide synthesis is likely going to be one that has been trained using data from a similar process. In the context of the present disclosure, a similar process may refer to a process that uses the same protection chemistry, the same activators, the same number of equivalents per coupling, the same instrument, the same or similar concentrations of reagents (such as e.g. concentrations within an order of magnitude, within 100%, within 50%, or within 10% of each other), the same detection system (e.g. the same type of HPLC, and/or the same approach to analysing HPLC traces), and/or the same additives (such as e.g. oxyma). A first and second processes may be considered to be similar or sufficiently similar for the purpose of the present disclosure when the prediction of the outcome of production of peptides using a model as described herein trained on data set obtained from a first process achieves a predetermined prediction accuracy for a second process. The predetermined prediction accuracy may be defined in terms of e.g. AUC or MAE. The predetermined prediction accuracy may be set to a level that is sufficient for a particular application, such as e.g. an MAE of at most 20%, at most 15% or at most 12%, or an AUC of at least 0.6, at least 0.65 or at least 0.7. The predetermined accuracy may be set to a level that corresponds to the level that can be achieved for a process that is identical to the process from which training data was obtained, or to a level within a set tolerance of said level (e.g. within 10% of the accuracy achievable for the same process). A first and second processes may be considered to be similar or sufficiently similar for the purpose of the present disclosure when a first and second model trained for the prediction of the outcome of peptide production as described herein for the first and second processes, respectively, use the same predictive features and/or the same ranking of predictive features (where a ranking of predictor features ranks predictive features by their predictive importance). This does not however mean that a model trained on data from a different process will not be able to provide useful predictions, it merely underlines the fact that feature selection and importance may depend on the particular process and thus the invention is not limited to the exact features and coefficients provided in Table 3. Further, the success of the models in predicting in particular purity (but also pass rate, to some extent) with features importance that vary between manufacturers and does not necessarily align with “common wisdom” underlines the limitations of existing approaches based on simple rules and intuitions.

TABLE 3
Coefficients of trained linear models for the prediction of peptide purity and
pass/fail. Aggregation = Average aggregation parameter of a sequence, P_{agg},
from Kranach et al. 1993; aromaticity = aromaticity according to Lobry, 1994;
instability index = instability index according to Guruprasad et al 1990;
flexibility = flexibility according to Vihinen, 1994; gravy = GRand
AVerage of hydrophobicitY according to Kyte and Doolittle; Helix = fraction
of amino acids which tend to be in Helix (V, I, Y, F, W, L); turn = fraction
of amino acids which tend to be in Turn (N, P, G, S); sheet = fraction of
amino acids which tend to be in sheet (E, M, A, L); mec = Molar extinction
coefficient; retention time = chromatographic retention time.

| Feature | MFG1 purity | MFG1 pass | MFG2 purity | MFG2 pass |
|---|---|---|---|---|
| Y intercept | 41.30110736 | 0.172481429 | 43.75325786 | 0.11602455 |
| Aggregation | −0.736308358 | −0.033175863 | −1.464678009 | −0.091767837 |
| Isoelectric point | −7.79907E−05 | 0.116276406 | 0.313800944 | 0.108892452 |
| retention time | 0.128060514 | 0.012288415 | 0.160877761 | −0.011768409 |
| density | −0.426296692 | −0.108838776 | −0.630023412 | −0.013187567 |
| Aromaticity | 0.166359636 | 0.019911178 | 0.341376499 | −0.018213106 |
| Instability index | 0.025104289 | −0.043201788 | 0.369064371 | −0.22924571 |
| Flexibility | 1.143058224 | 0.040445869 | 0.416033329 | 0.003146541 |
| Gravy | −0.653519382 | −0.04177836 | −0.612097004 | −0.052626698 |
| helix | 0.205321113 | 0.019589143 | −0.022437283 | −0.072063602 |
| turn | 0.659641641 | 0.087633021 | 0.895549598 | 0.099916929 |
| sheet | 0.076774854 | 0.145219468 | 0.44411628 | 0.104343537 |
| mec | 0.043493762 | −0.004714642 | 0.172482463 | −0.022246475 |
| Ala | 1.031414385 | 0.11955422 | 0.54371961 | 0.008745733 |
| Cys | −3.274855251 | −0.373412838 | −1.594897554 | 0.034346035 |
| Asp | 0.15947632 | 0.176396974 | 0.625629361 | −0.08540174 |
| Glu | −0.196745789 | 0.060285941 | 0.600128992 | 0.154203277 |
| Phe | 0.087325352 | 0.009559183 | 0.18035293 | −0.06119627 |
| Gly | 0.206977539 | 0.046609552 | 1.149728619 | −0.081991455 |
| His | 1.113950408 | 0.054952555 | 0.810689529 | 0.061550156 |
| Ile | 0.258448773 | −0.004806393 | −1.143804525 | −0.10938708 |
| Lys | 1.253769247 | 0.160992473 | 0.279490754 | 0.017757518 |
| Leu | 0.068972271 | 0.093501668 | 0.258186354 | −0.023765982 |
| Met | −1.663474437 | −0.079903379 | −1.252633098 | 0.085061706 |
| Asn | −0.105404792 | −0.090368272 | −0.086010292 | 0.025279528 |
| Pro | 2.299042668 | 0.309533912 | 3.935452656 | 0.356489719 |
| Gln | 0.204382468 | 0.025913729 | −0.639543044 | −0.078970672 |
| Arg | −1.193890171 | −0.354314303 | −1.336821802 | −0.153390569 |
| Ser | −1.457141851 | −0.170213203 | −3.352277838 | −0.124805536 |
| Thr | −0.87903188 | −0.16165168 | −0.704845057 | −0.071354328 |
| Val | −0.057506786 | −0.100978425 | 0.203646517 | −0.008524005 |
| Trp | −0.037526567 | −0.019762884 | 0.03879745 | −0.055449969 |
| Tyr | 0.217172869 | 0.037711735 | 0.358542783 | 0.076426768 |

FIG. 7B shows the weights for the predictive features in trained models for pass/fail prediction for two manufacturers (MFG 1, MFG2) for pass/fail classification models (balanced logistic regression and logistic regression, respectively top and second row for each manufacturer) and purity prediction regression models. The data illustrate that there are some differences in the weights learned for the two manufacturers, although the trends (positive/negative predictors of manufacturability) are generally aligned. A negative weight for a feature indicates that the feature is associated with difficulty in manufacturing, and a positive weight indicates that the feature is correlated with manufacturing success.

Based on the above, a number of experiments were tried to improve performance of the regression and classifier, using different datasets. Manufacturing failures were removed from the training set. For one manufacturer, data from the other was added. Finally, failures were removed in addition to the other manufacturer being added. Cross validation was performed by batch to account for batch effects, as above. Peptides that failed manufacturing are not included in the error calculation to account for the bimodal residuals as discussed above. For the regression models, paired sample t-tests (between batches) showed improvement over baseline (“All Data”) when only training on passes (“Passes Only”); MAE=11.7% vs 10.4%, p=0.00. For the MFG 1 model, including the other manufacturer makes the model better but this gives no incremental benefit over removing failed peptides; MAE=10.4% vs 10.5%, p=0.99. For the MFG 2 model, removing failed peptides makes things better (MAE=13.3% vs 12.1%, p=0.01) but adding in MFG 1 makes things worse (MAE=12.1% vs 12.7%, p=0.01). This is believed to be because the MFG1 model uses features unused by MFG 2 so their inclusion makes the model less effective (while the reverse is not true). For the classifiers, for MFG 1, inclusion of data from the other manufacturer does not have a significant effect; AUC=69.3% vs 69.3%, p=0.44. For MFG 2 this makes things worse; AUC=65.6% vs 61.3%, p=0.01.

In order to study whether a batch effect is visible in the results of the model as well as in the raw data, the regression residual standard deviations per batch for the ridge regression model trained on the entire MFG 1 training set was examined alongside the purity standard deviation per batch. A similar approach was used for the classification model, looking at failure rate by batch and false positive/negative rates in the classification. The results of this are shown on FIG. 10. On FIG. 10, the top plot shows, for each batch, the purity standard deviation in the batch (left) and the standard deviation of the corresponding residual of the regression model (right)—i.e. the variance that is unexplained by the model. This showed large batch to batch variability in the unexplained variance, with some batches being very well explained by the model and others being harder to predict. A similar conclusion is visible on the bottom plot of FIG. 10, which shows for each batch the failure rate per batch (left), the classification false positive rate (middle) and the classification false negative rate (right). Linear mixed effect models (Lindstrom & Bates, 1988) were investigated where the intercept of the model and one or more additional features were allowed to vary by batch. However, no significant improvement in the model, or reductions in unexplained variance was obtained. Some batches were still found to have higher or lower residual variation (regression) and false positive negative rate (classification) than one would expect, as assessed using the approach explained above in relation to FIG. 5. This indicates that batches may be subject to complex sources of variability making some batches more difficult to explain than others. In turn, this indicates that batch-specific parameters (i.e. process parameters) would likely enable to entangle some of these effects, resulting in an improvement of the predictive capability of the models.

### Active Learning

Active learning is a machine learning approach to improving models when data is expensive to acquire but there is some freedom to choose data points to be acquired (Settles, 2012). An approach where multiple data points are collected at once (i.e. adding a batch of data) as set out in Pinsler et al. (2019) was used. Specifically, an optimisation problem was laid out involving maximising the likelihood of a model, having selected a subset of at most b points from a possible selection of M points. Several Active Learning scenarios were looked at to model choosing a new batch in this way. Starting with a small (100), medium (500) or large (1000) point dataset, 10, 100 or 200 more points were chosen from a possible set of 1000 (or 2400 in one case). These scenarios were modelled for both synthetic data and the MFG 1 regression data (real purity data). Synthetic data was generated using a Ridge model to correspond to the Ridge model that is being fitted. In this case, the Ridge model fitted was a linear regression model with a prior on the parameters coming from a Gaussian with mean 0 and a set precision (inverse variance) of 0.05. Thus, the synthetic data was obtained by creating a toy model by drawing coefficients from the prior (in this example 60 parameters were used), generating some data with this model and adding noise to the data generated.

Observations of this model were linear in the parameters and had residual variance of 350.

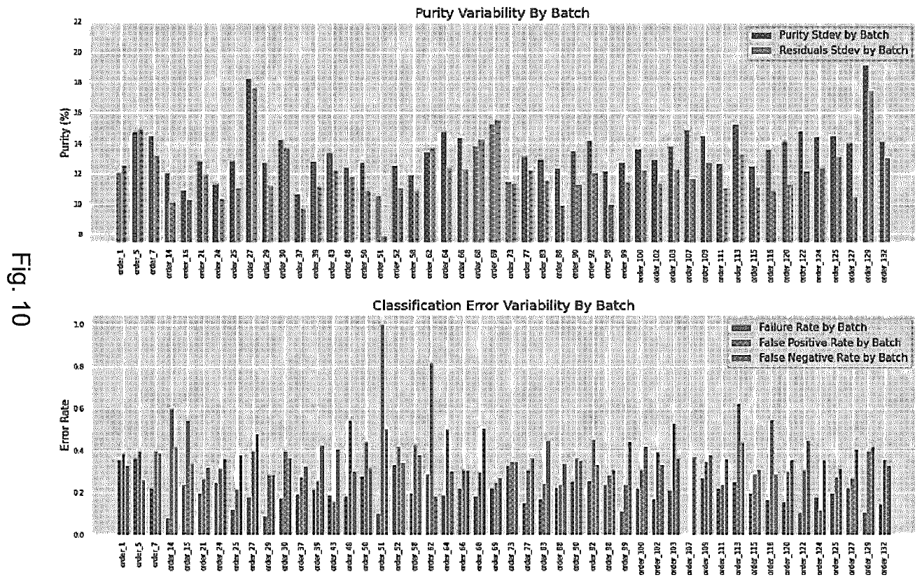

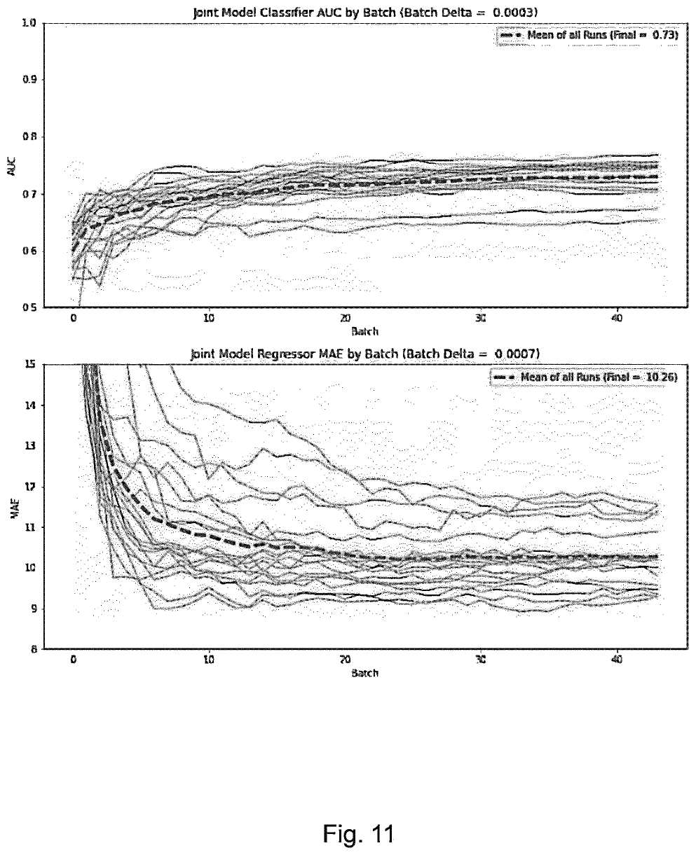

Then, the regression scores that can be obtained by fitting the corresponding Ridge regression model to the synthetic data using batch active learning was assessed. Results are shown in Table 4. Regression R² scores are reported before a batch is selected, then the change in score is also reported after a batch of randomly selected data is added, and after a batch of data selected with active learning is added. This data shows that active learning has a big impact when there is not much data available to train the models, but as more data becomes available, not only is the improvement over randomly selecting the data smaller, but the overall change is smaller too. Nevertheless, the approach demonstrates that there is a benefit to using additional data, with a particularly big jump between 100 and 200 observations, that active learning may be particularly useful to select this additional data when the data set is small, but that there are diminishing returns to the approach when a critical amount of data is already present (such as e.g. at least 500 data points). This is further shown on FIG. 11 which shows the learning curves for Ridge and Logistic Regression models (AUC and MAE by batch), using MFG1 data. The effect of adding batches incrementally to a training set is studied by investigating model performance on a validation set. In particular, for each of 20 runs, three batches from the training set were chosen as validation. For the remaining batches, each was added one at a time to a training set, the models were trained on these, then performance was calculated on the validation set. Each run is a green line and the average over all runs is a blue line. Batch to batch variability is evident as some green lines are worse than others—these have “difficult” batches in their validation sets. However, all lines are flattening. This means the marginal improvement to the linear models is small for each new batch. This indicates that the amount of data available is sufficient for the problem solved, and that models trained on even smaller data sets (e.g. 10 or more batches, at least 500 peptides, preferably at least 1000 peptides) are likely to perform well.

TABLE 4
Active Learning results.

| | | | | | | +Active |
|---|---|---|---|---|---|---|
| | Start | Batch | Selection | Base | +Random | learning |
| Data Type | Data | data | pool | (%) | (%) | (%) |
| Synthetic | 100 | 10 | 1000 | 33 | 7.3 | 11.1 |
| Synthetic | 100 | 100 | 1000 | 38 | 29.3 | 31.0 |
| Synthetic | 500 | 10 | 1000 | 74 | 0.0 | 0.1 |
| Synthetic | 500 | 100 | 1000 | 74 | 0.6 | 0.7 |
| Synthetic | 1000 | 200 | 1000 | 76 | 0.3 | 0.3 |
| Synthetic | 1000 | 200 | 2400 | 75 | 0.2 | 0.2 |
| Real | 100 | 10 | 1000 | −53 | 8.8 | 12.3 |
| Real | 100 | 100 | 1000 | −51 | 43.7 | 45.7 |
| Real | 500 | 10 | 1000 | 13 | 0.2 | 0.3 |
| Real | 500 | 100 | 1000 | 14 | 2.0 | 2.8 |
| Real | 1000 | 200 | 1000 | 19 | 1.0 | 1.0 |
| Real | 1000 | 200 | 2400 | 19 | 0.7 | 0.9 |

### Unsupervised Feature Learning

Another approach to improving models is to add new predictive features. This can be done automatically, using Unsupervised Learning or Transfer Learning. Several unsupervised approaches are studied here, in particular deep probabilistic autoencoders (a sequential VAE, a sequential WAE and a sequence-level VAE) as well as transfer learning using features from a pretrained transformer model. Briefly, this indicated that the most effective approaches to identify new informative feature as the use of sequential models, indicating that sequential effects are at play. Further, the models were also used to investigate whether the particular peptides in the data available are significantly different from random peptides (which may bias the results of the studies), by comparing latent representations of peptides from the models to latent representations of comparative peptides. This was found not to be the case. These models are all practically similar to a standard autoencoder, except with a regularisation term appearing as a KL divergence or a MMD distance (see methods). These penalise the network if it learns a posterior different to N(0, 1). The models are therefore disincentivised from memorising the training data completely in the encoding network, making them less likely to overfit the data. As explained above, a bidirectional transformer model was also tested to generate features from an intermediate encoder stack. This was not found to perform significantly better than the autoencoders, though it may be useful for longer peptides.

Several versions of the NVDM, VLSTM and WLSTM were looked at. Size of encoder and decoder as well as latent dimensions were varied, as well as regularisation strength. Each of these models was used to obtain latent features that were added to the features described above in relation to the linear models, and new regression and classification models using both the curated and unsupervised features were trained and evaluated. Tables 5-7 report these results. Encoder and decoder size, which for the NVDM refers to the size of the encoding and decoding MLPs, and for the VLSTM and WLSTM refer to the number of hidden states in the encoding and decoding LSTMs, was varied from 64 to 1024 dimensions. The latent dimensions were varied from 32 to 512. Broadly, an encoder/decoder size of 256 was representative of the larger sizes and likewise 128 for hidden dimensions. For the V/WLSTMs, a hidden size of 0 was additionally looked at to check the incremental effect of including hidden states. For all the networks, unregularised encoders are also reported, to check the effect of regularisation. The WLSTM unregularised autoencoders are the same as their VLSTM counterparts. For the NVDM, reconstruction log likelihood was reported, as well as average log of the variance of the test set in latent space, MAE of a regression including parameters learned by the model and AUC of a logistic regression. For the regression and classification, results for MFG 1 only are reported. For MFG2, no useful features were learned (see below), but this was thought to be due to a lack of data. For V/WLSTM, reconstruction distance was reported instead of likelihood, which is the average Levenshtein distance (Levenshtein, 1966) of a test set of proteome peptides reconstructed by the model. This Levenshtein distance is the edit distance between the predicted and actual peptides (i.e. the minimum number of single character edits required to change one sequence into the other). Random sequences 29 residues long would be on average 25 units apart by this measure.

TABLE 5
NVDM autoencoders. Model architecture and corresponding
unsupervised metrics (Reconstruction Log Likelihood and Latent Log
Variance). MAE and AUC on regression and classification tasks,
using the features described above in relation to the linear models
andthe learned features from the NVDM model, are also provided.
All models' MAE and AUC should be compared with base-line,
using only the hand crafted features, of MAE 10.4% and AUC 69.0%.

| Hidden | | | Reconstruction | Latent | | |
|---|---|---|---|---|---|---|
| dims | Z dims | KL Max | Log Like | Variance | MAE | AUC |
| 64 | 32 | 0 | −640.72 | −19.23 | 10.69 | 0.70 |
| 64 | 32 | 1 | −530.98 | −2.02 | 10.69 | 0.70 |
| 256 | 128 | 0 | −648.84 | −26.88 | 10.75 | 0.70 |
| 256 | 128 | 1 | −506.99 | −1.01 | 10.73 | 0.70 |

For the NVDM, Table 5 shows that increasing network capacity and regularisation improves the reconstruction likelihood. The encoder log variance is increased by regularisation, indicating that regularisation is working. However, the features learned by this model do not improve over baseline for regression or classification. Small differences in regression MAE are observed, possibly due to batch-to-batch variability and/or to overfitting of the training data. The differences are small indicating that at least similar performances could be obtained for example by optimising the regularisation process for example by optimising the number of epochs (early stopping process).

TABLE 6
VLSTM autoencoders. Model architecture and
corresponding unsupervised metrics (Reconstruction
Distance on a test set and Latent Log Variance), as well
as MAE and AUC on regression and classification tasks.

| Hidden | | | Test | Latent | | |
|---|---|---|---|---|---|---|
| dims | Z dims | KL Max | reconstruction | Variance | MAE | AUC |
| 64 | 0 | 0 | 23.82 | | | |
| 64 | 32 | 0 | 15.00 | −19.41 | 10.56 | 0.74 |
| 64 | 32 | 0.5 | 18.91 | −1.62 | 10.53 | 0.72 |
| 256 | 0 | 0 | 25.00 | | | 0.74 |
| 256 | 128 | 0 | 6.27 | −16.23 | 10.52 | 0.74 |
| 256 | 128 | 1 | 12.71 | −0.67 | 10.60 | 0.73 |

For the VLSTM, the data in Table 6 shows that addition of latent states enable the network to reconstruct more effectively. Regularisation impairs reconstruction performance. The networks all learn useful features and regularisation does not improve the features learned. Similar conclusions apply to the WLSTM (Table 7), however the latent log variances are low for this network, indicating it is close to deterministic, even with regularisation. It was not possible to train networks with higher log variance for the WLSTM.

TABLE 7
WLSTM autoencoders. Model architecture and
corresponding unsupervised metrics (Reconstruction
Distance on a test set and Latent Log Variance), as well
as MAE and AUC on regression and classification tasks.

| | | | | Test | | | |
|---|---|---|---|---|---|---|---|
| Hidden | Z | σ | | recon- | Latent | | |
| Dim | dim | kernel | λ | struction | Variance | MAE | AUC |
| 64 | 32 | 0.1 | 10 | 15.80 | −17.49 | 10.58 | 0.71 |
| 256 | 128 | 0.1 | 10 | 8.98 | −15.36 | 10.48 | 0.75 |

Thus, this data indicates that regression was not improved by the addition of latent features from deep learning networks (although it was also not significantly worse, indicating that the latent features may still be useful for other data sets—in particular if the variability observed between repeat batches could be untangled e.g. using process parameters). This further indicates that the linear models described above may have captured all explainable sources of variation. However, the sequential networks were able to learn useful information for the classification problem (see Table 8 below which shows an improved AUC with the LSTM and WSTM for an independent test data set). This is further investigated on FIG. 14 which shows the cross-validation performance of a L2 regularised logistic regression using expert defined features and learned features from various unsupervised AI models. The wide distribution in these violin plots is caused by batch effects. The “standard” model refers to a L2 regularised logistic regression classifier with only the expert defined features described in the Linear models section. Features from other models were then added to these features. The smallest transformer from Rives et al. (2021) was also used to generate features. This was achieved by downloading the pretrained model and passing MFG1 peptides through it. Paired sample t tests suggest that the VLSTM, WLSTM and transformer features all add something over baseline. They yield an AUC of 73%-74% and the t-tests are significant with p=0.00. However, the data indicates that combining these feature sets together does not improve performance. So, in additional to the expert defined features described above, there are additional features that have been discovered by the models in this section. There is likely significant overlap between the features discovered by different models as none offers incremental benefit over any other. The data indicates that these additional informative features are likely sequential in nature because they have only been found by the sequential models.

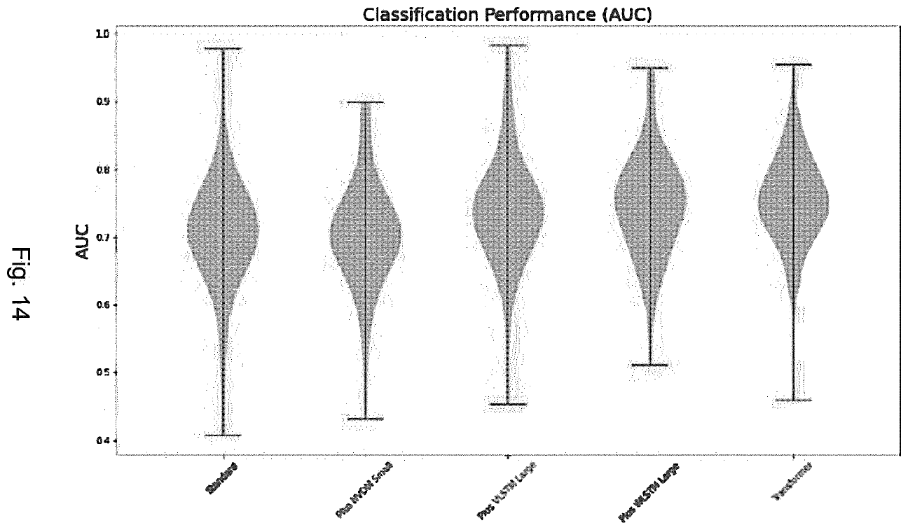

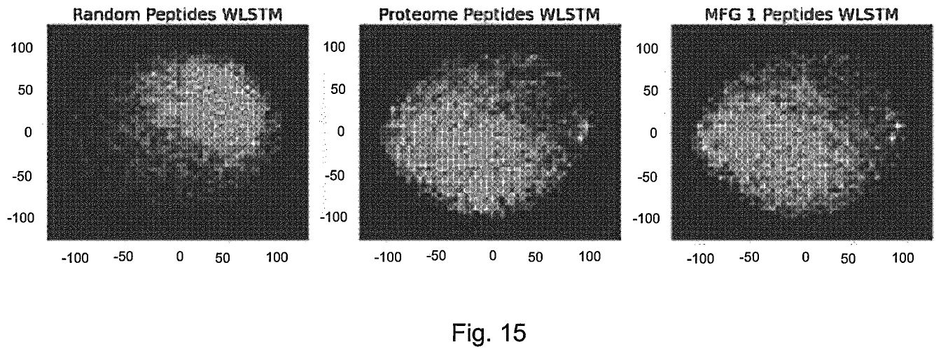

The peptides in the data were also compared with completely random peptides and peptides randomly selected from the peptidome in the space of latent variables learned from the models. These three sets of peptides were encoded by the largest regularised WLSTM tested above using the network that makes the posterior means. These encodings were projected in two dimensions using tSNE (Van der Maaten & Hinton, 2008) and heatmaps were drawn. The results of this are shown on FIG. 15 and indicate that the model learned features that differentiates the proteome and MFG1 peptides from the random peptides, and looked similar to each other with no evident clustering. This indicates that the model sees the peptides in the experimental data and those drawn from the proteome as coming from the same distribution, indicating that the choice of the random proteome as a training set was appropriate for this case. Colouring the MFG1 peptides in this space using output characteristics purity and pass/fail did not show any clear pattern. However, colouring these peptides using some of the expert defined features such as gravy and % proline revealed focal areas with higher values. This indicates that the autoencoder is learning known (expert defined features) in addition to unknown ones, as can be expected for a model that correctly learns sequence-based features. Similar results were seen for the VLSTM and NVDM (not shown).

### Neural Supervised Models

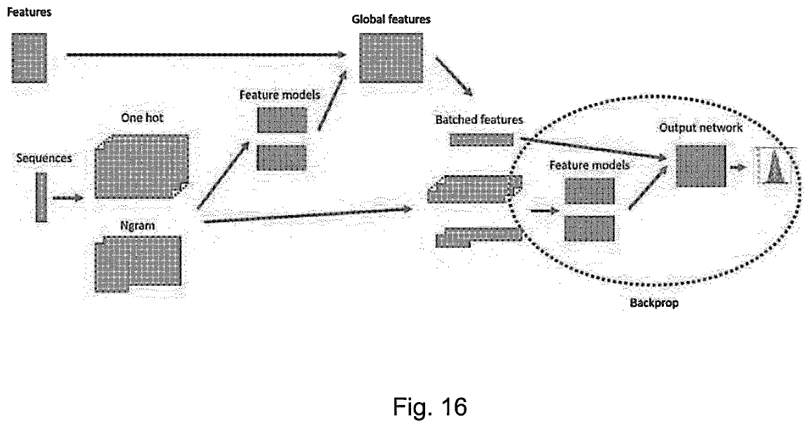

In this section, models that capture non-linear effects as well as a sequence model trained in a supervised way were tested using a common framework illustrated schematically on FIG. 16. A neural framework that can be easily configured to study non-linear interactions, pretrained features, or supervised training of sequence models. This framework can accept preconstructed features such as those used in the Linear Models section as well as models that generate features, for example the WLSTM from the previous section. The features used are generated on the fly and combined, prior to feeding into a regression or classification output network. This network may have one layer, in which case it is a linear model, or multiple layers with non-linear activations between, in which case it is non-linear. Moreover, networks that generate features from sequence data can be learned at training time. So, a LSTM with no prior training can be introduced; its parameters are learned in a supervised way as part of the regression or classification task.

Firstly, a model with global features only and linear output networks, leading to similar regression and classification networks to those already studied. These models were trained to convergence after 20 epochs for the classifier and 40 for the regression. A non-linear version of this model was studied, with a 50-node linear layer, followed by a ReLU activation, and then a 10 node layer, reducing to a single node, optionally with a sigmoid activation for the classifier. Both were stopped early at 20 epochs. Two feature models were also tested. A WLSTM was used to generate features from sequences to feed into the global features. This had a hidden state size of 256, a latent state size of 128, a kernel width of 0.1 and a distance penalty of 10. A LSTM network was also used to generate features on the fly. This was randomly initialized and trained by the network, appearing in the Feature Models section inside the backpropagation envelope on FIG. 16. It had 32 hidden states. Training was stopped early after 20 epochs for the classifier and 5 for the regression, to improve performance.

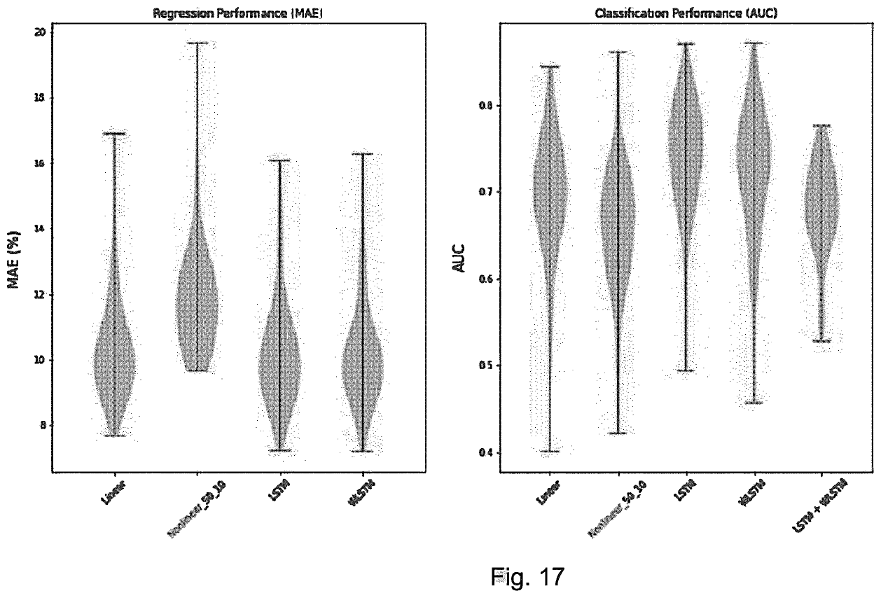

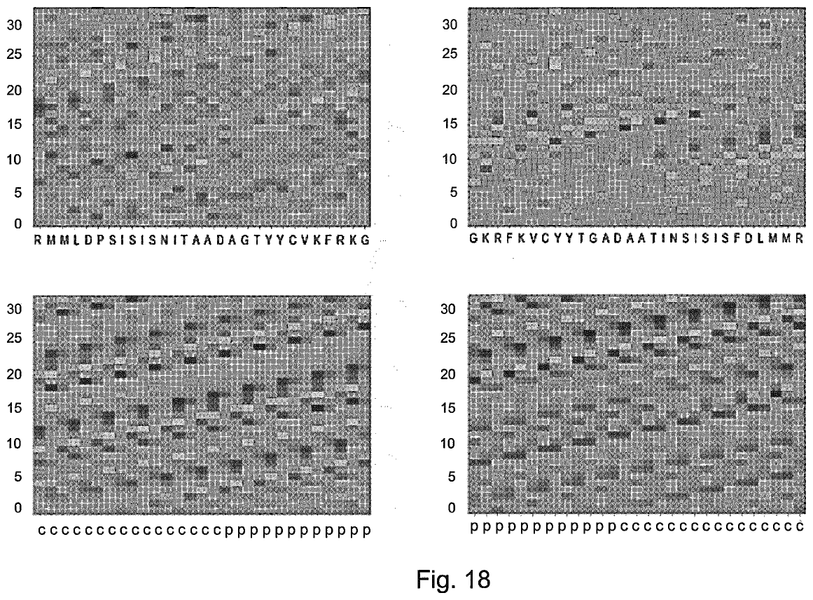

FIG. 17 and the first two columns of Table 8 show performance of these networks on the regression and classification tasks, for MFG 1. The non-linear output network makes performance worse for both regression (11.94% vs 10.41% average MAE, p=0.00) and classification (66% vs 69% average AUC, p=0.00). Both the WLSTM and LSTM networks make classification performance better (72% and 74% average AUC respectively, p=0.00 for both). We would expect this from the WLSTM given the results in the previous section. That the trained LSTM network does better is interesting. Combining these two approaches, both generating global features from the WLSTM and training an LSTM, seems to make things worse (67% average AUC, p=0.00). Performance of the LSTM vs the WLSTM is comparable (p=0.23). All p-values are for paired sample t-tests, where the samples are the batches. The last two columns of Table 8 show performance of the networks using MFG 2 data. Now, none of the non-linear models or pretrained features give higher performance. This is likely to be because of the smaller dataset for MFG 2. FIG. 18 illustrates the operation of the trained LSTM network, indicating that sequential information is being learned. In particular, the figures show the activations of LSTM models in the classification task. On the x axis is the amino acid at each point in the sequence. Along the y axis are the 32 hidden feature activations at each node of the LSTM. The top left plot shows network output for a representative sequence. There are clear, increasing lines of activation from left to right, indicating the network is learning about the position of the input amino acids. Reversing the order of the sequence (top right) yields different features, indicating that order matters. This reflects our understanding that difficult amino acid appearing at the start of the sequence have a different importance to ones appearing at the end. The bottom left plot shows a manually constructed sequence starting with 14 Prolines followed by 15 Cystines. This plot has stronger activations, which is a consequence of the repeated nature of the sequence. Reversing this sequence (bottom right) again yields a different plot. Thus, the regression task was once again not improved by the use of more complex models and features, bit the classification task could be improved using sequential model features trained as part of a neural network framework. The features learned by these models are likely to be similar to those learned by the unsupervised models described above, as no combination of these models resulted in better performance than any one alone.

TABLE 8
Performance of neural models in regression
and classification tasks.

| | MFG1 | MFG1 | MFG2 | MFG2 |
|---|---|---|---|---|
| | regression | classifier | regression | classifier |
| | MAE | AUC | MAE | AUC |
| Linear | 10.4% | 69% | 12.1% | 66% |
| Nonlinear | 12.0% | 68% | 15.6% | 59% |
| LSTM | 10.3% | 74% | 12.8% | 51% |
| WLSTM | 10.3% | 73% | 12.0% | 64% |
| LSTM + WLSTM | | 67% | | 60% |

### Test on Holdout Batches

All the regression/classification performances provided above relate to cross-validation results. As a final test of the conclusions obtained, a set of models were tested using holdout batches (i.e. unseen “Test” batches) and an additional batch that was obtained at a later time (“New Batch”). This represents a rigorous test of the models. In particular, a linear regression with the expert defined features described above, a linear classifier using learned LSTM features, a classifier using pretrained WLSTM features and a non-linear classifier with the expert defined features were tested. Table 9 shows the results of this for the MFG1 data and Table 10 shows the results of this for the MFG2 data. Training and testing results showed comparative performance for both MFG1 and MFG2, indicating that both sets of models were trained robustly (without overfitting). The LSTM classifier improved performance in the MFG1 data compared to the linear classifier with expert features only. This was not true for the MFG2 data, probably due to the small size of this data set. MFG 2 is also harder to model in the non-linear setting, possibly also because of less data. The marginal improvement of the LSTM model over the linear model is the same for training and test data.

TABLE 9
Out of sample results for MFG 1 peptides.

| | Train | Test | New batch |
|---|---|---|---|
| Linear regression MAE | 10.4% | 11.1% | 11.9% |
| Linear classifier AUC | 69.3% | 73.9% | 74.2% |
| LSTM classifier AUC | 74.0% | 78.7% | 78.8% |
| WSTM classifier AUC | 73.2% | 77.1% | 77.4% |
| Nonlinear classifier AUC | 70.9% | 67.6% | 72.6% |

TABLE 10
Out of sample results for MFG 2 Long peptides

| | Train | Test |
|---|---|---|
| Linear regression MAE | 12.1% | 14.7% |
| Linear classifier AUC | 66.0% | 63.4% |
| LSTM classifier AUC | 56.0% | 56.9% |
| WSTM classifier AUC | 69.3% | 60.5% |
| Nonlinear classifier AUC | 74.1% | 63.2% |

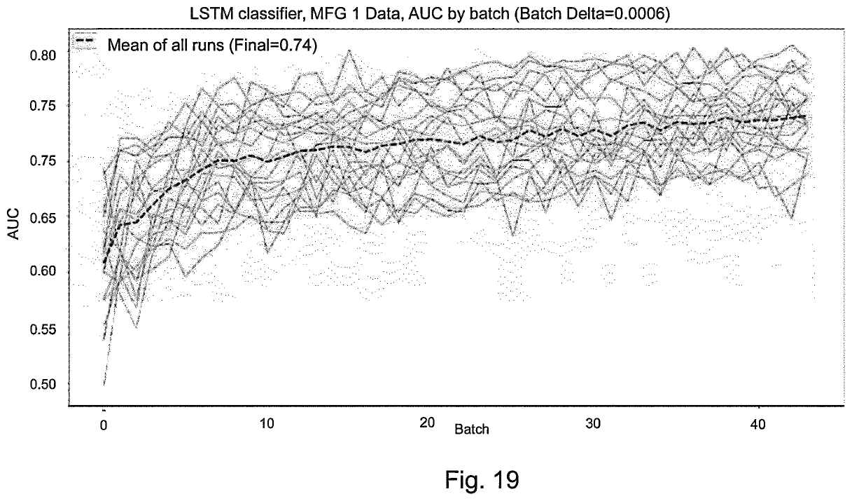

FIG. 19 shows the learning curve for the LSTM model using MFG 1 data. Once again, 20 runs have been performed. On each run, three batches from the training set were used as validation. For each curve in the plot, a new training set was made batch-by-batch and performance (on the validation set) of the model trained on these was recorded. The dashed line is the average of these runs. The average performance is steadily increasing which means this model could benefit from more data. So, with 20 more batches the AUC might increase from 73% to 76%. When the linear regression was trained using data that had failed manufacturing, test performance was made worse. For the MFG 1 data it went from 11.1% to 14.5% and for MFG 2 it went from 14.7% to 14.9% (both p=0.0, paired sample t-test).

### Simulated Improvements in Manufacturing

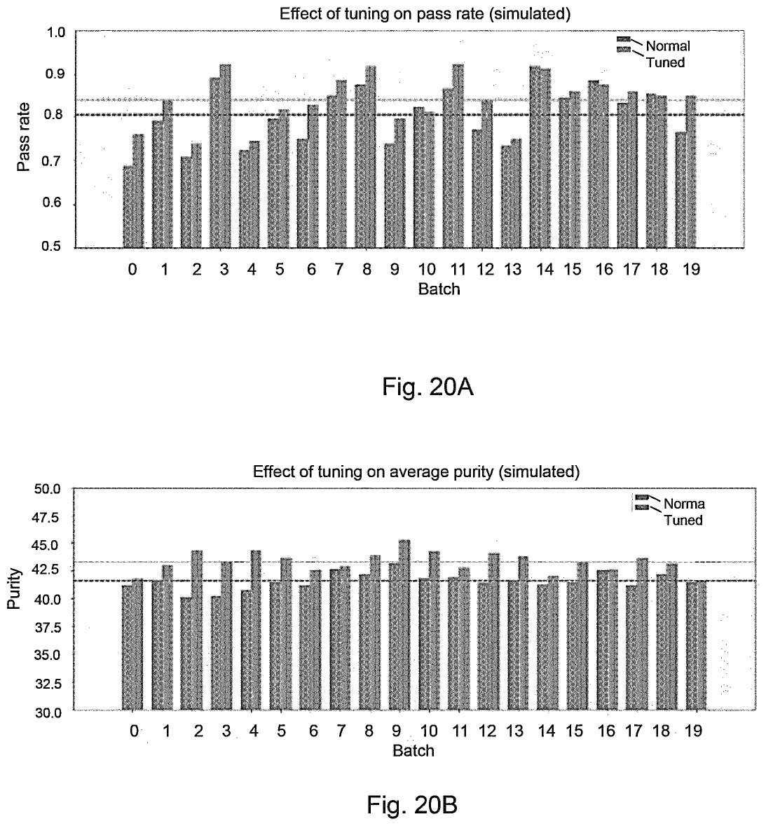

A simulation of how these models could be used to improve the outcome of synthesis of desired peptides was performed. In this simulation, it is assumed that for every peptide, it is possible to design an alternative desired peptide that contains mostly the same sequence, shifted by a few positions. For example, a desired peptide may be one that contains a mutation present in the tumour of a patient, but there may be some flexibility in relation to the location of the mutated position in the peptide. Thus, a candidate peptide may be designed with the mutation at a default position, and based on predictions from the models described herein, one or more alternative peptides comprising the mutation at a different position may be proposed. In this example the peptides were assumed to be of constant length, i.e. the peptides include a frame of constant size around the mutated position that, and that frame is shifter along the proteome sequence while still including the mutated position. However, this does not need to be the case and the peptides could instead be cropped on either or both sides of the mutated position. Peptides were drawn randomly from the human proteome and then tuned, by shifting up to five peptides either side (i.e. up to five amino acid positions toward the C-ter or the N-ter). They were tuned to maximise the predicted pass probability. The effect of this tuning was then simulated. Tuned and normal peptides were assigned pass or fail based on the model. Then, errors were introduced to reflect the assumption that real pass/fail rates are likely to depart from predictions from the model (due to imperfect model performance). Model pass and fail error rates (e.g. number of correctly chosen passes divided by number of passes) were then applied to these realisations. These error rates were drawn from the observed distribution of model error rates, as described above. Then, average pass rates of the normal and tuned batches were recorded. FIG. 20A shows a typical realisation of 20 simulated batches (each group of bars is one batch, left bar is normal pass rate—Mean 0.81, right bar is tuned pass rate—Mean=0.84). An analogous experiment was carried out for purity, where the errors introduced were from the observed batch residual distributions (FIG. 20B—each group of bars is one batch, left bar is normal purity—Mean=, right bar is tuned purity—Mean=43.30). The data on FIGS. 20A and 20B shows that an increase in pass rate and purity can be obtained using this method, as also shown in the data in Table 11 where different lengths of shifts were tested. In embodiments, the shifting used may not be constant across peptides, for example a different shift may individually be selected for each peptide within predetermined boundaries (such as e.g. up to 3, 5 or 10 amino acids). A shift may be selected for a peptide based on the predictions from the model, and optionally one or more additional rules. For example, shift values may be selected to avoid particular amino acids (e.g. cysteine) when located near enough to a terminus of the peptide to be excluded by shifting.

TABLE 11
Effect of tuning on peptides from MFG1

| | Normal | Tuned | | |
|---|---|---|---|---|
| | pass | pass | Normal | Tuned |
| Shift | rate | rate | purity | purity |
| 10 | 81% | 85% | 41.2% | 44.0% |
| 5 | 80% | 83% | 41.2% | 43.2% |
| 3 | 80% | 83% | 41.1% | 42.5% |

Depending on the context and in particular the intended use of the peptide, the freedom to shift the peptide sequence may be limited or non-existent. Conversely, depending on the context and in particular the intended use of the peptide, it may be possible to otherwise modify the peptides to improve the predicted outcome of manufacture, such as e.g. by trimming the peptides (removing one or more amino acids at either or both ends of the peptide, such as e.g. up to 3, 5 or 10 amino acids) and/or by substituting the amino acids at one or more positions for equivalent amino acids (where equivalence can be determined by a set of rules provided by a user). Further, all possible modifications may be evaluated in isolation or in combination, such as e.g. a combination of shifting and trimming. Thus, the methods described herein may be used for many other purposes beyond identifying shifted peptides with improved pass rates and/or purity corresponding to a candidate set of peptides. For example, the methods could be used to prioritise peptides for manufacture, based on the predicted outcome of synthesis (e.g. pass likelihood and/or predicted purity). For example, a set of peptides to be manufactured could be selected from a set of candidate peptides based on a criterion applying to the predicted outcome of synthesis and optionally one or more further criteria (such as e.g. a target set size, a criterion applying to the importance of one or more peptides, etc.). This could enable to focus synthesis resources on peptides that are likely to be synthesised to a desired standard, such as e.g. to a desired purity. A desired purity may be chosen to be sufficient for the intended use, or sufficient for subsequent purification steps to be implemented in such as way as to lead to a purity that is sufficient for the intended use. Indeed, purification steps may result in loss of material such that there may be a level of purity (or other outcome of the peptide synthesis process such as the identity of the impurities) that is such that purification will not lead to sufficient amounts of product at a desired purity. As another example, the methods could be used to identify peptides that are to be manufactured using a specific process out of one or more possible processes. For example, respective models could be trained to predict the outcome of peptide synthesis using different processes, and peptides could be selected for production with a process for which the predicted outcome meets a predetermined criterion (e.g. the process for which the predicted outcome is most advantageous, e.g. the process with the highest pass likelihood and/or highest predicted purity). As another example, the methods could be used to identify peptides that are to be manufactured using an adapted process for peptides predicted to be difficult to manufacture. For example, an adapted process using a different synthesiser may be used (e.g. a microwave synthesiser instead of a plate synthesiser), and/or an adapted process using higher concentrations of coupling reagents, and/or an adapted process using higher equivalents of amino acids to peptide and/or an adapted process using higher temperatures in one or more reaction steps. Without wishing to be bound by theory, it is believed that this would have the advantage of using a standard efficiency process for the peptides that can be successfully synthesised in this way, and using alternative/more time and/or resource consuming processes only when warranted (i.e. when a peptide is unlikely to be successfully synthesised in the standard process). As another example, an adapted process for peptide predicted to be difficult to manufacture may comprise one or more purification steps. Further, the choice of the one or more purification steps may depend on the predicted outcome of the peptide synthesis. As another example, the methods described herein could be used to identify batches of peptides or subsets of batches of peptides that may have been subject to manufacturing error, i.e. for quality control. Thus, the methods described herein could also be used to analyse peptides obtained by chemical synthesis after they have been produced. For example, batches of peptides where one or more measured metrics indicative of the outcome of synthesis are significantly different from the corresponding predicted metrics obtained using the present method (particularly if the measured metrics indicate a worse outcome than predicted) may be flagged for repeat and/or exclusion without requiring further extensive analysis to verify the outcome of the synthesis. In particular, while LC or LC-MS are currently performed after production of peptides, obtaining a particular metric for a peptide such as e.g. purity as assessed using these techniques cannot indicate whether the peptide is simply difficult to synthesise or whether the purity obtained was low because of a problem with the manufacturing process. This distinction may be relevant for example in making a decision as to whether to repeat the synthesis using the same process, or whether to abandon synthesis of the peptide or attempt synthesis using an adapted process. Comparing the metrics obtained with the predicted metrics may assist in making this decision. This is particularly the case when all or most peptides in a batch fail to reach predicted outcome metrics, as this likely indicates a problem with the batch rather than a characteristic of individual peptides. Further, techniques such as visualisation in latent space or visualisation in a space of predictive features as described herein using a data dimensionality reduction and visualisation technique (such as e.g. principal component analysis, PCA) may be used to verify that the peptides in the batch do not intrinsically differ significantly from those on which the models were trained. This may further increase the confidence in an assessment of whether a batch may be faulty as opposed to comprising more difficult peptides than expected.

### Monitoring Manufacturing Performance

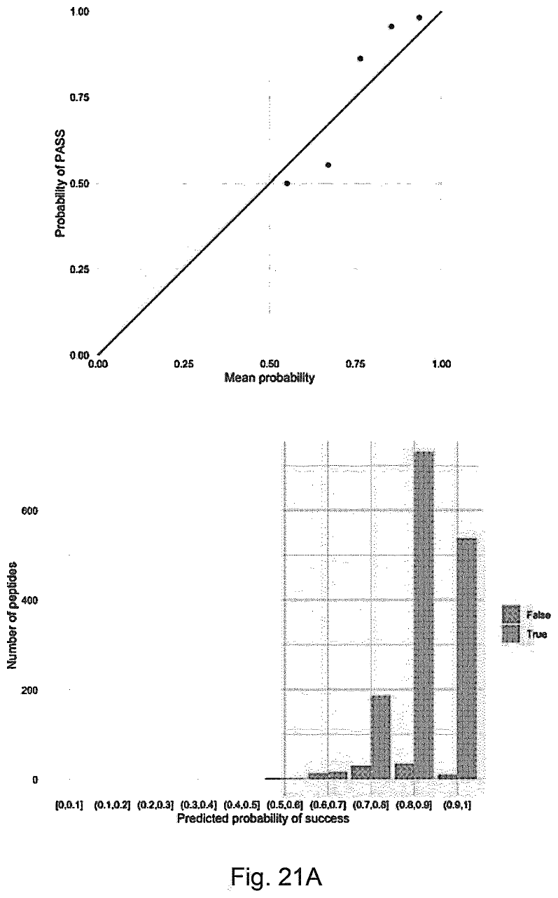

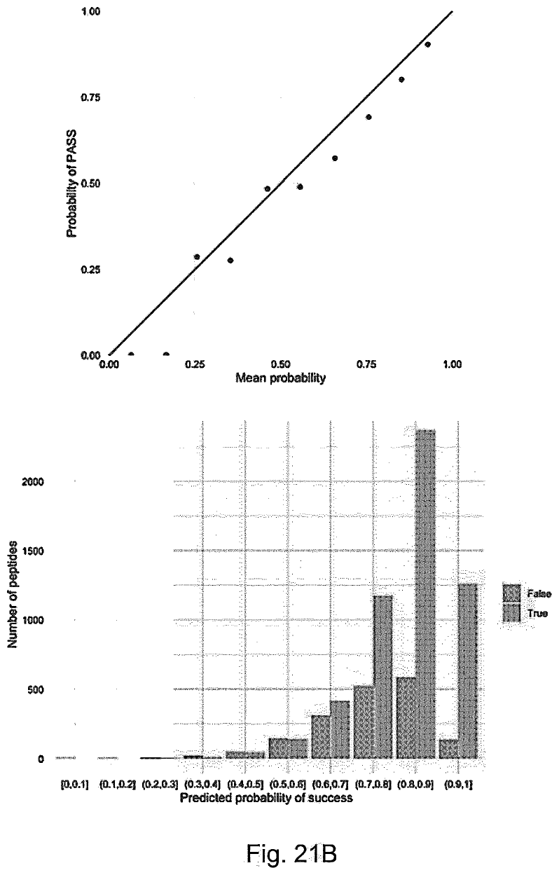

The use of the manufacturer-specific classification (Logistic regression; prediction of pass/fail) model described above in monitoring manufacturing performance was then investigated. First, the model calibration was verified by binning the predicted pass probabilities output by the classification model and verifying that the average predicted pass rate in a bin correlates with the average observed pass rate in the bin, using the independent test dataset used above. The results of this are shown on FIG. 21A (MFG1) and FIG. 21B (MFG2). This shows that for both manufacturers, there was a good correlation between the actual and predicted pass/fail rates, indicating that the models were adequately calibrated. This further indicates that on average, peptides that are predicted as more likely to be manufacturable (pass) are indeed more likely to be manufacturable, giving confidence in use of the predictions to rank/prioritise peptides for manufacture based on likelihood of success.

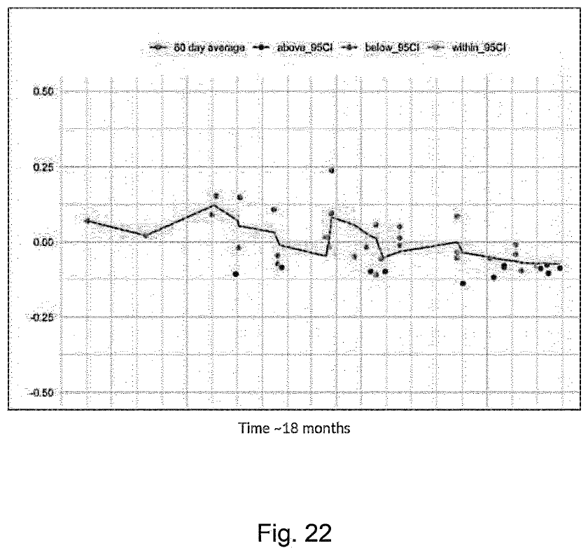

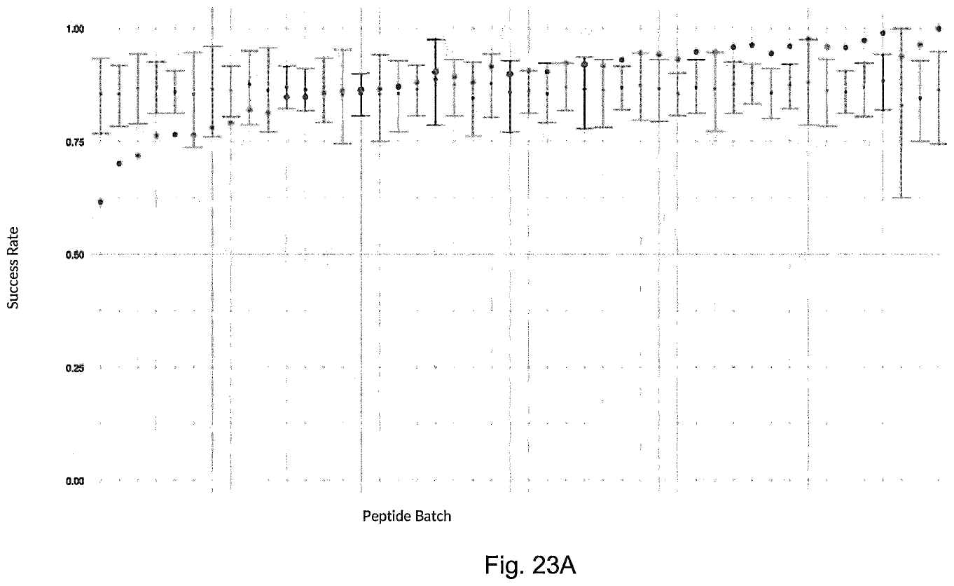

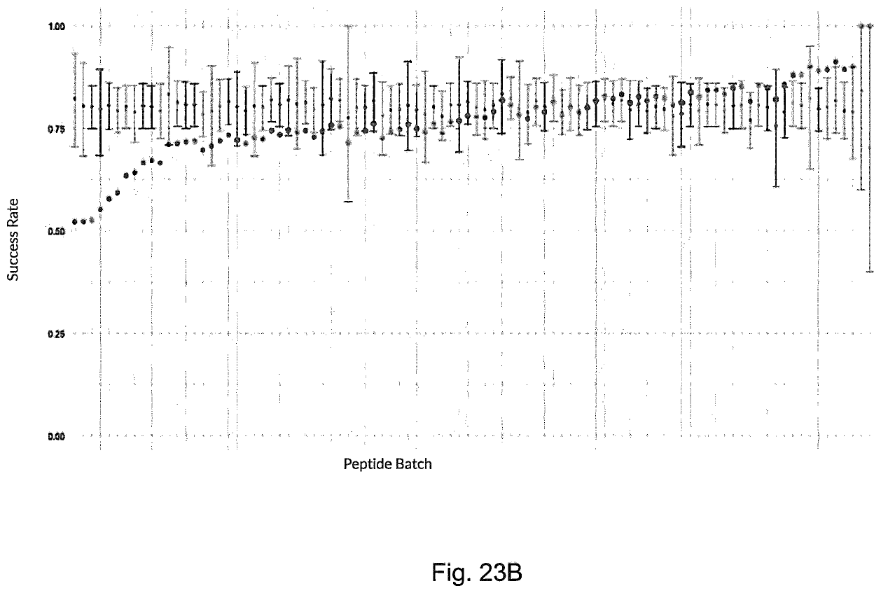

Given that these models were shown to be reliable, they can be used to monitor performance of a manufacturing process over time, by determining the deviation between observed and predicted pass rates for batches manufactured using the process over time (FIG. 22), and comparing actual and predicted success rate of peptide batches to identify outlier batches that may have suffered from machine errors, operator errs, reagent issues or any other spurious cause of manufacturing performance degradation (FIG. 23).

The results on FIG. 22 show that for this particular manufacturing process, the observed pass rate gradually decreased in comparison to the predicted pass rate (i.e. the difference between observed and predicted pass rate was negative and increased in absolute value over time), indicating a degradation in performance of the process. The use of the model to quantify this enables to account for possible differences in the actual peptides that are being manufactured in every batch (i.e. ensuring that reduced pass rates are not due to batches having increasing amounts of peptides that are difficult to manufacture). Instead, a increasing difference in observed vs. predicted pass rate indicates a genuine change in the performance of the process from the expected performance which characterises a baseline performance (defined by the performance achieved in data that was used to train the model). In this example, significant deviations from the predicted pass rate were determined by calculating the 95% confidence interval for a test comparing two binomial proportions, where the 95% confidence interval the range of differences where we can say that the proportions (predicted vs observed pass proportions) are truly different.

The results on FIG. 23 show that the models described herein can be used to identify individual batches that have unexpectedly low pass rates. These ay have occurred due to human errors, machine errors, reagents errors of defects, etc. Identifying these enables to exclude potentially lower quality batches, identify potential sources of errors in the process (e.g. by identifying common points between these batches such as use of the same particular piece of equipment, batch of reagent, operator, etc.), and ultimately remedy those for consistent/improved performance.

Independent Validation with “Difficult” Peptides

A set of peptides identified in Krchnak et al., 1993 as difficult to manufacture (prone to side-reactions/degradation) were analysed using one of the manufacturer-specific regression models described above, to verify that the model was able to predict purity for peptides independently selected specifically for manufacturing difficulty rather than the patient derived peptides that were used for training the models. These predictions were compared with observed purity when the peptides were manufactured by the manufacturer whose data was used to train the model. The results of this are shown in Table 11. Only 1 of the 10 peptides could be successfully synthesised by the manufacturer (according to the manufacturer's success criteria which may exclude extremely low purity results as failure to manufacture). The model was able to predict that all of these sequences would be difficult to synthesise (i.e. if the peptide could be synthesised at all, synthesis would be at extremely low purity—such low purities would likely be considered manufacturing failures).

TABLE 11
Predicted vs observed purity for “difficult”
peptides from Krchnak et al., 1993. Observed
purity = 0% for all peptides that were
reported by manufacturer as not successfully
manufactured.

| | | Observed | Predicted |
|---|---|---|---|
| | | Purity | Purity |
| Sequence | GRAVY | (%) | (%) |
| SCCQTTCCRTTCCRH | −0.02 | 0 | 0.02 |
| SCCVSSCCRPQCCQ | | | |
| GPCRRLHRRRRHLCC | −0.58 | 0 | 0.02 |
| RCCRCCCCCCWGRP | | | |
| QGLGQESCCRPSCCL | −0.02 | 0 | 0.06 |
| TTCCRTTCCRPSCC | | | |
| RGRPWGRRRRREQQR | −2.89 | 0 | 0.08 |
| GGRRRRCEWRRRQL | | | |
| RTTCWKPTTVTTCSI | 0.25 | 0 | 0.09 |
| TPCCQPSCCVSSCC | | | |
| SRRSRSQSSSRSRSC | −2.13 | 0 | 0.14 |
| GRRRSGNRDRTVQS | | | |
| CCTSPGGDQFCRSRW | −0.16 | 26 | 0.16 |
| TQWNLVIWCQCCVR | | | |
| LPSRARSRRPGRRPR | −1.21 | 0 | 0.17 |
| RRTCCTTTITTLPS | | | |
| LVRTRSCSSSMTSAR | −0.16 | 0 | 0.18 |
| CCCHSYTCPPSCPT | | | |
| GWISRCTCRTPSIGR | −0.60 | 0 | 0.19 |
| TWCPRAAGRCTRTP | | | |

### DISCUSSION

These examples demonstrate the development and assessment of new machine learning models for predicting the outcome of manufacturing of peptides by chemical synthesis (in particular, solid phase peptide synthesis using a batch method). This work has several innovations which significantly improve performance over previous solutions. In particular, the work demonstrated that despite some inherent unpredictability in the outcome of peptide synthesis which appears to be due to experimental factors that are difficult to control (as evidenced by the strong batch effects and large variability between repeat batches), it was possible to obtain strongly predictive models using solely features derivable from the primary sequence of the peptides to be synthesised. The work further shows that expert defined features alone can be used to produce models with high predictive power, and that these could be further improved using unsupervised deep learning models that are able to learn sequential effects. The work additionally indicates that process features such as the identity of the reagents used, concentrations, temperature, instrument identity etc. likely contribute to the batch variability observed and that as such models including such features would likely have improved predictive power. Further, the models could also be used to optimise a production process, for example by identifying process variables that are predictive of process outcome. Thus, also described herein is a method for optimising or designing a peptide production process, the method comprising training a model as described herein using one or more process parameters as input features, and identifying process parameters that are predictive of the outcome of production of peptides. The effect of these parameters on the production outcome could be investigated based on the coefficients of the model, for example in the case of a linear regression or classification model. This could in turn be used to identify one or more specific values for these process parameters that are likely to result in improved production outcomes.

The models provided herein improve on both simple single metrics methods like that in Milton et al. and complex deep learning methods like those in Mohapatra et al. Indeed, the methods described herein are expected to perform significantly better than single metric models since the linear model analyses demonstrated that multiple sequence-derived features can bring complementary information (i.e. while there is some redundancy in the set of features explored, a combination of multiple features always provided the best predictions), and that the most informative features depend on the particular process used (in particular, models trained using data from two different manufacturers using different protocols and instruments did not show the same exact informative features although there was of course some overlap since both manufacturers used broadly similar SPPS approaches). Compared to complex models such as that in Mohapatra et al., the present methods have the advantage of being significantly simpler to train and run, and can make use of and be useful for prediction of the outcome of synthesis using SPPS in batch processes, which are by far more commonly available. The present methods could be used to predict the outcome of synthesis using any process including flow-based methods, as they are able to predict the final outcome of any synthesis. As the methods described herein predict final outcome, they would not make use of the data available in flow-based methods in relation to individual couplings during synthesis. Nevertheless, this may still be useful in situations where insufficient such data is available but prediction for a flow synthesis system is nonetheless desirable. Indeed, the relative leanness of the models described herein means that useful models can be trained with far fewer data points than models trained exclusively on flow-based data such as in Mohapatra et al. Additionally, the present work demonstrates a process whereby a set of peptides can be designed based on a set of mutations to be included in manufactured peptides, such that the set of peptides has an improved likelihood of synthesis success compared to a default set of peptides.

Finally, the present work demonstrates that the methods can be used to identify batches that have been subject to unexpected errors or problems in the synthesis process, and to monitor performance of a manufacturing process over time. This can be useful to identify drifts in performance that can be attributable to aging components of the synthesis process, or any other sources of degradation in performance, or conversely to monitor the effectiveness of measures implemented to maintain and/or improve performance of a peptide synthesis process.

### REFERENCES

Landau D A, Carter S L, Stojanov P, McKenna A, Stevenson K, Lawrence M S, Sougnez C, Stewart C, Sivachenko A, Wang L, Wan Y, Zhang W, Shukla S A, Vartanov A, Fernandes S M, Saksena G, Cibulskis K, Tesar B, Gabriel S, Hacohen N, Meyerson M, Lander E S, Neuberg D, Brown J R, Getz G, Wu C J. Evolution and impact of subclonal mutations in chronic lymphocytic leukemia. Cell. 2013 Feb. 14; 152(4):714-26. doi: 10.1016/j.cell.2013.01.019.
Lu Y C, Zheng Z, Robbins P F, Tran E, Prickett T D, Gartner J J, Li Y F, Ray S, Franco Z, Bliskovsky V, Fitzgerald P C, Rosenberg S A. An Efficient Single-Cell RNA-Seq Approach to Identify Neoantigen-Specific T Cell Receptors. Mol Ther. 2018 Feb. 7; 26(2):379-389.
Leko V, McDuffie L A, Zheng Z, Gartner J J, Prickett T D, Apolo A B, Agarwal P K, Rosenberg S A, Lu Y C. Identification of Neoantigen-Reactive Tumor-Infiltrating Lymphocytes in Primary Bladder Cancer. J Immunol. 2019 Jun. 15; 202(12):3458-3467.
Hundal J, Kiwala S, Feng Y Y, Liu C J, Govindan R, Chapman W C, Uppaluri R, Swamidass S J, Griffith O L, Mardis E R, Griffith M. Accounting for proximal variants improves neoantigen prediction. Nat Genet. 2019 January; 51(1):175-179. doi: 10.1038/s41588-018-0283-9.
Roth A, Khattra J, Yap D, Wan A, Laks E, Biele J, Ha G, Aparicio S, Bouchard-Côté A, Shah S P. PyClone: statistical inference of clonal population structure in cancer. Nat Methods. 2014 April; 11(4):396-8. doi: 10.1038/nmeth.2883.
McGranahan N, Furness A J, Rosenthal R, Ramskov S, Lyngaa R, Saini S K, Jamal-Hanjani M, Wilson G A, Birkbak N J, Hiley C T, Watkins T B, Shafi S, Murugaesu N, Mitter R, Akarca A U,
Linares J, Marafioti T, Henry J Y, Van Allen E M, Miao D, Schilling B, Schadendorf D, Garraway L A, Makarov V, Rizvi N A, Snyder A, Hellmann M D, Merghoub T, Wolchok J D, Shukla S A, Wu C J, Peggs K S, Chan T A, Hadrup S R, Quezada S A, Swanton C. Clonal neoantigens elicit T cell immunoreactivity and sensitivity to immune checkpoint blockade. Science. 2016 Mar. 25; 351(6280):1463-9. doi: 10.1126/science.aaf1490.
Simon, M. D., Heider, P. L., Adamo, A., Vinogradov, A. A., Mong, S. K., Li, X., Berger, T., Policarpo, R. L., Zhang, C., Zou, Y., Liao, X., Spokoyny, A. M., Jensen, K. F. and Pentelute, B. L. (2014), Rapid Flow-Based Peptide Synthesis. ChemBioChem, 15: 713-720.
Mohapatra, Somesh, et al. “Deep Learning for Prediction and Optimization of Fast-Flow Peptide Synthesis.” ACS central science 6.12 (2020): 2277-2286.
Milton, RC de L., Saskia C F Milton, and Paul A. Adams. “Prediction of difficult sequences in solid-phase peptide synthesis.” Journal of the American Chemical Society 112.16 (1990): 6039-6046.
Chou, Peter Y., and Gerald D. Fasman. “Empirical predictions of protein con-formation.” Annual review of biochemistry 47.1 (1978): 251-276.
Cock P A, Antao T, Chang J T, Chapman B A, Cox C J, Dalke A, Friedberg I, Hamelryck T, Kau F, Wilczynski B and de Hoon M J L (2009) Biopython: freely available Python tools for computational molecular biology and bioinformatics. Bioinformatics
Goloborodko, A. A.; Levitsky, L. I.; Ivanov, M. V.; and Gorshkov, M. V. (2013)\Pyteomics—a Python Framework for Exploratory Data Analysis and Rapid Software Prototyping in Proteomics”, Journal of The American Society for Mass Spectrometry, 24(2), 301-304.
Pedregosa et al., Scikit-learn: Machine Learning in Python, JMLR 12, pp. 2825-2830, 2011.
McKinney, Wes. “Data structures for statistical computing in python.” Proceedings of the 9th Python in Science Conference. Vol. 445. 2010.
Seabold, S., Perktold, J. (2010). statsmodels: Econometric and statistical modeling with python. In 9th Python in Science Conference.
Paszke, A., Gross, S., Massa, F., Lerer, A., Bradbury, J., Chanan, G., . . . Chintala, S. (2019). PyTorch: An Imperative Style, High-Performance Deep Learning Library. In Advances in Neural Information Processing Systems 32 (pp. 8024-8035).
Harris, C. R., Millman, K. J., van der Walt, S. J. et al. Array programming with NumPy. Nature 585, 357-362 (2020).
Vihinen, Mauno, Esa Torkkila, and Pentti Riikonen. “Accuracy of protein flexibility predictions.” Proteins: Structure, Function, and Bioinformatics 19.2 (1994): 141-149.
Guruprasad, Kunchur, BV Bhasker Reddy, and Madhusudan W. Pandit. “Cor-relation between stability of a protein and its dipeptide composition: a novel approach for predicting in vivo stability of a protein from its primary sequence.” Protein Engineering, Design and Selection 4.2 (1990): 155-161.
Lobry, J. R., and Christian Gautier. “Hydrophobicity, expressivity and aromaticity are the major trends of amino-acid usage in 999 Escherichia coli chromosome-encoded genes.” Nucleic acids research 22.15 (1994): 3174-3180.
Krchnak, V., Z. Flegelova, and Josef Vagner. “Aggregation of resin-bound pep-tides during solid-phase peptide synthesis. Prediction of difficult sequences.” International journal of peptide and protein research 42.5 (1993): 450-454.
Narita, Mitsuaki et al. “Prediction and improvement of protected peptide solubility in organic solvents.” International journal of peptide and protein research 24.6 (1984): 580-587.
Buchan D W A, Jones D T (2019). The PSIPRED Protein Analysis Workbench: 20 years on. Nucleic Acids Research. doi.org/10.1093/nar/gkz297
McGuffin, L. J., Atkins, J., Salehe, B. R., Shuid, A. N. & Roche, D. B. (2015) IntFOLD: an integrated server for modelling protein structures and functions from amino acid sequences. Nucleic Acids Research, 43, W169-73
Lambert C, Leonard N, De Bolle X, Depiereux E. ESyPred3D: Prediction of proteins 3D structures. Bioinformatics. 2002 September; 18(9):1250-1256
Minkyung Baek, Frank DiMaio, Ivan Anishchenko, Justas Dauparas, Sergey Ovchinnikov, Gyu Rie Lee, Jue Wang, Qian Cong, Lisa N. Kinch, R. Dustin Schaeffer, Claudia Millen, Hahnbeom Park, Carson Adams, Caleb R. Glassman, Andy DeGiovanni, Jose H. Pereira, Andria V. Rodrigues, Alberdina A. van Dijk, Ana C. Ebrecht, Diederik J. Opperman, Theo Sagmeister, Christoph Buhlheller, Tea PavkovKeller, Manoj K Rathinaswamy, Udit Dalwadi, Calvin K Yip, John E Burke, K. Christopher Garcia, Nick V. Grishin, Paul D. Adams, Randy J. Read, David Baker. Accurate prediction of protein structures and interactions using a 3-track network. Science 10.1126/science.abj8754 (2021)
Söding J. (2005) Protein homology detection by HMM-HMM comparison. Bioinformatics 21, 951-960.

Waterhouse, A., Bertoni, M., Bienert, S., Studer, G., Tauriello, G., Gumienny, R., Heer, F. T., de Beer, T. A. P., Rempfer, C., Bordoli, L., Lepore, R., Schwede, T. SWISS-MODEL: homology modelling of protein structures and complexes. Nucleic Acids Res. 46(W1), W296-W303 (2018).
Kyte J, Doolittle R F (1982) A simple method for displaying the hydropathic character of a protein. J Mol Biol 157: 105-132.
Lindstrom, Mary J., and Douglas M. Bates. “Newton-Raphson and EM algorithms for linear mixed-effects models for repeated-measures data.” Journal of the American Statistical Association 83.404 (1988): 1014-1022
Das, Payel, et al. “Accelerating antimicrobial discovery with controllable deep generative models and molecular dynamics.” arXiv preprint arXiv:2005.11248 (2020).
Tolstikhin, Ilya, et al. “Wasserstein auto-encoders.” arXiv preprint arXiv:1711.01558 (2017).
Kingma, Diederik P., and Max Welling. “Auto-encoding variational bayes.” arXiv preprint arXiv:1312.6114 (2013).
Miao, Yishu, Lei Yu, and Phil Blunsom. “Neural variational inference for text processing.” International conference on machine learning. PMLR, 2016.
Pruitt, Kim D., Tatiana Tatusova, and Donna R. Maglott. “NCBI reference sequences (RefSeq): a curated non-redundant sequence database of genomes, transcripts and proteins.” Nucleic acids research 35.suppl 1 (2007): D61-D65.
Bowman, Samuel R., et al. “Generating sentences from a continuous space.” arXiv preprint arXiv:1511.06349 (2015).
Rives, Alexander, et al. “Biological structure and function emerge from scaling unsupervised learning to 250 million protein sequences.” Proceedings of the National Academy of Sciences 118.15 (2021).
Devlin, Jacob, et al. “Bert: Pre-training of deep bidirectional transformers form language understanding.” arXiv preprint arXiv:1810.04805 (2018).
Levenshtein, Vladimir I. “Binary codes capable of correcting deletions, insertions, and reversals.” Soviet physics doklady. Vol. 10. No. 8. 1966.
Van der Maaten, Laurens, and Geoffrey Hinton. “Visualizing data using t-SNE.” Journal of machine learning research 9.11 (2008).
Rives, Alexander, et al. “Biological structure and function emerge from scaling unsupervised learning to 250 million protein sequences.” Proceedings of the National Academy of Sciences 118.15 (2021).
David Ha, Douglas Eck, A Neural Representation of Sketch Drawings. arXiv:1704.03477. May 2017.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only and are not to be construed as limiting the disclosure in any way.

The methods of any embodiments described herein may be provided as computer programs or as computer program products or computer readable media carrying a computer program which is arranged, when run on a computer, to perform the method(s) described above. Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise. The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

1. A method comprising:
providing a primary amino acid sequence for one or more peptides, and
predicting the outcome of production of the one or more peptides by chemical synthesis using a machine learning model that has been trained to predict one or more metrics characterising the outcome of production of peptides using training data comprising training peptide primary sequences and measured metrics characterising the outcome of production of the training peptides by chemical synthesis, wherein the measured metrics are metrics obtained after completion of the chemical synthesis process.

2. The method of claim 1, wherein the machine learning model takes as input:
one or more features derived from the primary structure of peptides, optionally wherein the machine learning model takes as input the value of one or more process parameters, and/or
one or more features selected from a set of candidate features using a feature selection process, optionally wherein the feature selection process comprises using a regularisation technique and/or removing features that show a correlation in the training data above a predetermined threshold,
optionally wherein the one or more features are standardised prior to being used as input to the machine learning model.

3. The method of claim 2, wherein the features derived from the primary structure of a peptide are selected from: features quantifying the amino acid composition of the peptide, features indicative of the secondary structure of the peptide derivable from its primary structure, features indicative of the propensity of the peptide to aggregate during manufacture, features associated with the behaviour of the peptide in solution, features indicative of physico-chemical properties of the peptide, and features learned by a peptide sequence model.

4. The method of claim 3, wherein:
features quantifying the amino acid composition of the peptide comprise the percentage or proportion of each of one or more amino acids or groups of amino acids,
features indicative of the secondary structure of the peptide derivable from its primary structure comprise the predicted proportion or percentage of amino acids in alpha helixes, beta chains and/or turns, and scores quantifying the propensity for amino acids in a chain to form alpha helixes, beta chains and/or turns, optionally wherein the scores quantifying the propensity for amino acids in a chain to form alpha helixes, beta chains and/or turns comprise the Chou-Fasman parameters Pₐ, P_{b} and Pₜᵤᵣₙ,
features indicative of the propensity of the peptide to aggregate during manufacture comprise scores that aggregate amino acid based metrics quantifying likelihood of aggregation, optionally wherein the amino acid based metrics are empirical metrics and/or wherein the features indicative of the propensity of the peptide to aggregate during manufacture comprise the aggregation parameters P_{agg} and P*_{c},
features associated with the behaviour of the peptide in solution comprise metrics indicative of the solubility of the peptide, instability of the peptide, and flexibility of the peptide, optionally wherein a metric indicative of solubility is a summarised solubility score calculated based on amino acid specific solubility scores, or wherein a metric indicative of solubility is the average of the amino acid specific solubility scores S provided in Table 2, or wherein a metric indicative of the instability of a peptide is an instability metric obtained by summing empirical weight values associated with dipeptides in proteins and quantifying the impact of said dipeptides on protein stability, normalised by the length of the peptide, or wherein a metric indicative of the flexibility of a peptide is a metric based on average flexibility parameters over windows of a fixed length,
features indicative of physico-chemical properties of the peptide comprise the density, charge at a particular pH (e.g. charge at pH=7), isoelectric point, chromatographic retention time, a feature indicative of hydrophobicity, and molar extinction coefficient of the peptide, optionally wherein the feature indicative of hydrophobicity is the aromaticity or the gravy score, and/or
features learned by a peptide sequence model comprise features learned by one or more neural network models trained to learn encodings of unlabelled peptide data.

5. The method of any preceding claims, wherein the one or more peptides have a length of at least 12, 13, 14, 15, 16, 17, 18, 19 or 20 amino acids, optionally at least 15 amino acids, and/or wherein the one or more peptides have a length of at most 50, 45, 40, 35, 34, 33, 32, 31 or 30 amino acids, preferably at most 40 amino acids, and/or wherein the one or more peptides have a length between 15 and 40 amino acids or between 20 and 35 amino acids, and/or wherein the one or more peptides have a length within the same length boundaries as the peptides in the training data.

6. The method of any preceding claim, wherein the chemical synthesis is a solid phase peptide synthesis, optionally wherein the chemical synthesis is a batch process, and/or wherein the chemical synthesis for which the outcome is predicted uses a similar process to the chemical synthesis process used to produce the training data, optionally wherein a similar process is a process that uses the same instrument(s), instrument(s) of the same type, the same protection chemistry, the same activators, the same number of equivalents per coupling, the same concentrations of reagents, the same detection system, and/or the same additives.

7. The method of any preceding claim, wherein predicting the outcome of production of the one or more peptides by chemical synthesis comprises predicting the value of the one or more metrics characterising the outcome of production of the peptide, wherein the metrics characterising the outcome of production of the peptide are metrics derivable from a chromatographic analysis, optionally a LC or LC-MS analysis, of the composition resulting from the process of chemical synthesis of the peptide(s); and/or
wherein predicting the outcome of production of the one or more peptides by chemical synthesis comprises predicting the value of the one or more metrics characterising the outcome of production of the peptide selected from: the purity of the resulting composition, one or more features of one or more chromatographic peaks associated with the composition, the identity of one or more products of the composition, whether the composition satisfies one or more criteria that apply to said metrics, the probability that the composition satisfies one or more criteria that apply to said metrics, and metrics derived from said metrics combining features for multiple chromatographic peaks.

8. The method of any preceding claim, wherein predicting the outcome of production of the one or more peptides by chemical synthesis comprises predicting the purity of the composition resulting from the process of chemical synthesis of the one or more peptides, and/or predicting whether the purity satisfies one or more criteria, and/or predicting the probability that the purity satisfies one or more criteria, optionally wherein purity of a composition is defined for one or more target peptides in relation to a chromatogram of said composition as the percentage area of one or more chromatographic peak(s) in the chromatogram corresponding to the target peptides relative to the total area of the chromatogram, and/or wherein the one or more criteria comprise a minimum purity.

9. The method of any preceding claim, wherein the machine learning model comprises a regression model or a classification model, wherein the machine learning model comprises a linear model, wherein the machine learning model comprises a neural network, and/or wherein the machine learning model comprises a regularised model, optionally a L1 or L2 regularised linear regression or L1 or L2 regularised linear classification model, such as a regularised logistic regression model.

10. The method of any of claims 2 to 9, wherein the machine learning model takes as input the value of one or more process parameters, wherein process parameters are parameters that characterise how a particular chemical synthesis process is run, optionally wherein a process parameter is a parameter set by a user or measured prior to, during or subsequent to carrying out the process, and/or wherein one or more process parameters are selected from: the type, model or identifier of a synthesis instrument, the identity of an operator, the batch number of one or more reagents used to perform the synthesis, the value of one or more physico-chemical variables associated with the process, the value of one or more flows of solutions in the instrument, the presence or concentration of one or more activators, and the maintenance status of the instrument or any part thereof.

11. The method of any preceding claim, wherein the machine learning model takes as input one or more features learned by a peptide sequence model, wherein the peptide sequence model comprises one or more neural network models trained to learn encodings of unlabelled peptide data, optionally wherein the one or more neural network models are sequential models and/or wherein the one or more neural network models are deep neural networks and/or wherein the one or more neural network models are selected from: autoencoders and transformers.

12. The method of claim 11, wherein the one or more neural networks are autoencoders optionally selected from recurrent neural networks, long short term memory networks, variational autoencoders, neural variational document models (NVDMs), or Wasserstein autoencoders, and/or
wherein the one or more neural networks have been trained in an unsupervised manner using training data comprising at least 1000 peptides, at least 2000, at least 3000, at least 4000, at least 5000 peptides, or at least 10,000 peptides, or wherein the one or more neural networks have been trained as part of the supervised training of a neural network comprising the one or more neural networks generating encodings of unlabelled peptide data, the encodings being used as input to a neural network regressor or classifier trained to predict the one or more metrics characterising the outcome of production of peptides.

13. The method of claim 12, wherein the one or more neural networks have been trained in an unsupervised manner using training data comprising peptide sequences drawn from a collection of peptides and/or proteins from a reference sequence, peptide sequences drawn from a previously obtained data set, randomly sampled peptides, or combinations thereof.

14. The method of any preceding claim, wherein the machine learning model takes as input one or more features selected from the features listed in Table 3 or equivalents thereof, wherein the machine learning model takes as input a plurality of features selected from the features listed in Table 3 or equivalents thereof, wherein the machine learning model takes as input at least 5, at least 10 or at least 15 features listed in Table 3 or equivalents thereof, wherein the machine learning model takes as input substantially all of the features listed in Table 3 or equivalents thereof, wherein the machine learning model is a linear regression or logistic regression model taking as input the features listed in Table 3 and equivalents thereof, optionally wherein the model has the coefficients of any of the models listed in Table 3 or equivalent coefficients learned by fitting logistic regression or linear regression models to a particular training data set.

15. The method of any preceding claim, wherein the machine learning model takes as input one or more features quantifying the amino acid composition of the peptide, optionally wherein the features include one or more of the percentage or proportion of Cys, Pro, Ser, Arg and Met, optionally wherein the features include at least one of or all of the percentage or proportion of Cys, Pro and Arg.

16. The method of any preceding claim, wherein the machine learning model has been trained using cross-validation and/or wherein the machine learning model has been trained using a training data set comprising data for at least 1500, at least 2000, at least 3000, at least 4000, or at least 5000 peptides, optionally wherein the training data comprises data for a plurality of peptide production batches and cross-validation was performed in a manner that did not split data in the same batch between cross-validation training and test sets.

17. The method of any preceding claim, wherein the method comprises predicting the outcome of production of a plurality of peptides by chemical synthesis, and optionally further comprising ranking or otherwise prioritising the plurality of peptides using one or more of the predicted metrics characterising the outcome of production of the peptides; and/or wherein the method further comprises providing to a user, for example through a user interface, one or more results of the method, optionally comprising the one or more predicted metrics characterising the outcome of the peptide production and/or a value derived therefrom or associated therewith and/or the sequence of one or more peptides selected from a plurality of peptides for which an outcome of production has been predicted.

18. The method of any preceding claim, wherein the method comprises predicting the outcome of production by chemical synthesis of a plurality of candidate peptides, and selecting one or more of the candidate peptides that satisfy one or more predetermined criteria including at least one criterion that applies to a predicted metric characterising the outcome of producing the one or more candidate peptides, and/or
wherein the method comprises predicting the outcome of production by chemical synthesis of one or more candidate peptides and one or more peptides derived from the candidate peptides by shifting, trimming and/or substituting one or more positions in the sequence of the candidate peptides, and selecting one or more of the candidate peptides that satisfy one or more predetermined criteria including at least one criterion that applies to a predicted metric characterising the outcome of producing the one or more candidate peptides,
optionally wherein the at least one criterion that applies to the predicted outcome of producing the one or more candidate peptides is selected from: having a predicted purity that is above a predetermined threshold, having a predicted purity that is above a threshold set adaptively to select a predetermined number of candidate peptides with the highest predicted purity, and having a predicted purity that is above a threshold set adaptively to select a predetermined top percentile of candidate peptides, optionally wherein the predetermined number or top percentile of candidate peptides also satisfy one or more further criteria; and/or wherein selecting one or more of the candidate peptides comprises selecting the one or more candidate peptides for synthesis with a first or second process, and/or wherein selecting one or more of the candidate peptides comprises selecting the one or more candidate peptide for synthesis using a process that includes more purification steps, different reagent concentration(s), different temperatures, or a different chemistry from a process to be used for one or more peptides that are not selected.

19. The method of any preceding claim, wherein the method is computer implemented, wherein the method is a method for producing one or more peptides, wherein the method is a method for predicting the outcome of production of one or more peptides, or wherein the method is a method for selecting peptides for chemical synthesis.

20. A method for providing a tool for predicting the outcome of production of one or more peptides by chemical synthesis, the method comprising:
(i) obtaining a training data set comprising: training peptide primary sequences and measured metrics characterising the outcome of production of the training peptides by chemical synthesis, wherein the measured metrics are metrics obtained after completion of the chemical synthesis process; and
(ii) providing a machine learning model that predicts the values of the one or more metrics characterising the output of chemical synthesis of peptides in the training data.

21. A method of producing one or more peptides, the method comprising:
predicting the outcome of producing one or more candidate peptides using the method of any preceding claim;
selecting one or more of the candidate peptides that satisfy one or more predetermined criteria including at least one criterion that applies to the predicted outcome of producing the one or more candidate peptides; and
synthesising the one or more selected peptides.

22. A method of monitoring a process for chemical synthesis of peptides, the method comprising:
predicting the outcome of producing one or more peptides using the method of claims 1 to 19, thereby obtaining predicted values of one or more metrics characterising the outcome of production of the one or more peptides;
obtaining a measured value of one or more metrics characterising the outcome of production of the one or more peptides using the process, optionally wherein the obtaining comprises synthesising the one or more peptides and determining the value of the one or more metrics or providing previously measured values; and
comparing the measured values and the predicted values, optionally wherein deviation between the measured and predicted value is indicative of a deviation between the expected and observed performance of the process.

23. A method of providing an immunotherapy for a subject that has been diagnosed as having cancer, the method comprising:
obtaining a set of one or more candidate neoantigens for the subject, wherein the one or more candidate neoantigens were identified using a process comprising analysing one or more samples from the subject comprising tumour genetic material; and
designing an immunotherapy that targets one or more of the neoantigens identified, wherein the designing comprises predicting the outcome of production of one or more peptides identified for at least one of the candidate neoantigens using the method of any of claims 1 to 19,
optionally wherein the one or more neoantigens are clonal neoantigens and/or wherein the immunotherapy that targets the one or more of the neoantigens is an immunogenic composition, a composition comprising immune cells or a therapeutic antibody, and/or wherein the method further comprises producing one or more peptides selected from the identified peptides and/or producing an immunotherapy using one or more peptides selected from the identified peptides.

24. A method of treating a subject that has been diagnosed as having cancer, the method comprising administering an immunotherapy that has been provided using the method of claim 23, optionally wherein the method comprises providing the immunotherapy for the subject using the method of claim 23.

25. A system comprising:
a processor; and
a computer readable medium comprising instructions that, when executed by the processor, cause the processor to perform the steps of the method of any of claims 1 to 23.

26. One or more computer readable media comprising instructions that, when executed by one or more processors, cause the one or more processors to perform the steps of the method of any of claims 1 to 23.
